(19) United States
(12) Patent Application Publication
(10) Pub. No.: US 2011/0002855 A1
(43) Pub. Date: Jan. 6, 2011

(54) PIPERAZINYL OXOALKYL TETRAHYDRO-BETA-CARBOLINES AND RELATED ANALOGUES

(75) Inventors: Timothy M. Caldwell, Guilford, CT (US); Yang Gao, Madison, CT (US); Linghong Xie, Guilford, CT (US); Yuelian Xu, East Haven, CT (US)

Correspondence Address: CANTOR COLBURN LLP, 20 Church Street, 22nd Floor, Hartford, CT, 06103, US

(73) Assignee: NEUROGEN CORPORATION, Branford, CT (US)

(21) Appl. No.: 12/666,314

(22) PCT Filed: Jun. 25, 2008
(86) PCT No.: PCT/US08/68115

§ 371 Date: Sep. 21, 2010

Related U.S. Application Data
(60) Provisional application No. 60/945,959, filed on Jun. 25, 2007.

(51) Int. Cl. A61K 31/55; C07D 401/14; A61K 31/496; C07D 403/14; C07D 471/04; A61K 31/4985; A61K 31/437; C07D 413/14; A61K 31/5377; A61K 9/12; A61P 25/00; A61P 25/28; A61P 3/04; A61P 3/10

(52) U.S. Cl. 424/45; 544/361; 514/253.03; 544/294; 544/349; 514/249; 546/84; 514/292; 540/575; 514/218; 544/126; 514/232.8; 544/251; 544/238

(57) ABSTRACT

Piperazinyl oxoalkyl tetrahydro-beta-carbolines and related analogues of the formula (I): are provided, as are methods for their preparation and use. Such compounds may generally be used to modulate ligand binding to histamine H3 receptors in vivo or in vitro, and are particularly useful in the treatment of a variety of disorders in humans, domesticated companion animals and livestock animals. Pharmaceutical compositions and therapeutic methods are provided, as are methods for using such ligands for detecting histamine H3 receptors (e.g., receptor localization studies).

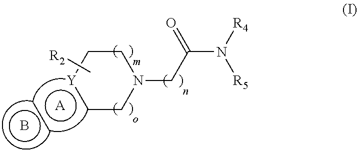

### CROSS-REFERENCE TO RELATED APPLICATIONS

This application claims the benefit of U.S. Provisional Patent Application Ser. No. 60/945,959 filed Jun. 25, 2007, which is incorporated by reference herein in its entirety.

### FIELD OF THE INVENTION

This invention relates generally to piperazinyl oxoalkyl tetrahydro-beta-carbolines and related analogues, and to the use of such compounds for treating conditions responsive to histamine H3 receptor modulation. The invention further relates to the use of such compounds as probes for the detection and localization of histamine H3 receptors.

### BACKGROUND OF THE INVENTION

Hormones and neurotransmitters regulate a wide variety of biological functions, often via specific receptor proteins located on the surface of living cells. Many of these receptors carry out intracellular signaling via the activation of coupled guanosine triphosphate-binding proteins (G proteins); such receptors are collectively called G protein-coupled receptors or GPCRs. The important role of GPCRs in the regulation of cell and organ function has attracted attention to these receptors as targets for new pharmaceutical agents.

Histamine is a multifunctional chemical transmitter that signals through specific cell surface GPCRs. To date, four histamine receptor subtypes have been identified: H1, H2, H3 and H4. Histamine H3 receptor is a presynaptic GPCR that is found primarily in the central nervous system, although lower levels are also found in the peripheral nervous system. Genes encoding the H3 receptor have been reported in various organisms, including humans (see Lovenberg et al. (1999) Molecular Pharmacology 55:1101-07), and alternative splicing of this gene appears to result in multiple isoforms. The histamine H3 receptor is an auto- and hetero-receptor whose activation leads to a decreased release of neurotransmitters (including histamine, acetylcholine, norepinephrine and glutamate) from neurons in the brain. Histamine H3 receptor is involved in the regulation of processes such as sleep and wakefulness, feeding and memory.

Antagonists of histamine H3 receptor increase synthesis and release of cerebral histamine and other neurotransmitters, inducing an extended wakefulness, an improvement in cognitive processes, a reduction in food intake and a normalization of vestibular reflexes. Such antagonists are useful, for example, as therapeutics for central nervous system disorders such as Alzheimer's disease, Parkinson's disease, schizophrenia, mood and attention alterations including attention deficit hyperactivity disorder and attention deficit disorder, memory and learning disorders, cognitive disorders (such as mild cognitive impairment and cognitive deficits in psychiatric pathologies), epilepsy, migraine, and disorders associated with the regulation of sleep and wakefulness, as well as in the treatment and prevention of conditions such as obesity, eating disorders, diabetes, vertigo, motion sickness and allergic rhinitis.

Accordingly, there is a need for new H3 receptor modulators. The present invention fulfills this need, and provides further related advantages.

### SUMMARY OF THE INVENTION

In certain aspects, the present invention provides piperazinyl oxoalkyl tetrahydro-beta-carbolines and related analogues of Formula I:

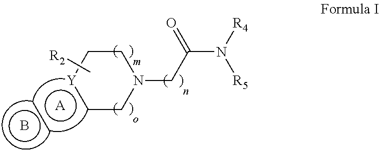

including the pharmaceutically acceptable salts, solvates (e.g., hydrates) and esters thereof.

### Within Formula I:

### Y is C or N;

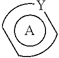

represents a 5- or 6-membered optionally substituted heteroaryl that is fused to the ring represented by

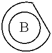

and is also fused to the ring represented by

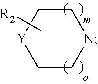

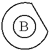

represents phenyl or a 5- or 6-membered heteroaryl that is fused to the ring represented by

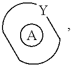

each of which phenyl or heteroaryl is optionally substituted and preferably substituted with from 0 to 4 substituents independently chosen from:
(i) hydrogen, amino, halogen, cyano, hydroxy, nitro and oxo; and
(ii) C₁-C₆alkyl, C₂-C₆alkenyl, C₂-C₆alkynyl, (C₃-C₈cycloalkyl)C₀-C₂alkyl, C₁-C₆alkoxy, C₁-C₆haloalkyl, C₂-C₆alkyl ether, C₁-C₆alkylsulfonyl, mono- or di-(C₁-C₆alkyl)aminoC₀-C₄alkyl, phenylC₀-C₂alkyl or (5- to 7-membered heterocycle)C₀-C₂alkyl; each of which is unsubstituted or substituted with oxo, C₁-C₆alkyl or C₁-C₆alkoxy;
   n is 0, 1, 2 or 3;
   m is 0, 1 or 2;
   o is 1 or 2;
R₂ represents from 0 to 4 substituents independently chosen from C₁-C₆alkyl (e.g., C₂-C₆alkyl or C₃-C₆alkyl), C₂-C₆alkenyl, C₂-C₆alkynyl, (C₃-C₈cycloalkyl)C₀-C₂alkyl, C₁-C₆haloalkyl and groups that are taken together to form a C₁-C₃alkylene bridge;
R₄ and R₅ are:
(i) independently chosen from C₁-C₆alkyl, C₂-C₆alkenyl, C₂-C₆alkynyl, C₁-C₆haloalkyl and C₂-C₆alkyl ether; each of which is substituted with from 0 to 4 substituents independently chosen from amino, cyano, oxo, mono- or di-(C₁-C₆alkyl)amino, mono- or di-(C₁-C₆alkyl)aminocarbonyl, and 5- to 7-membered heterocycloalkyl; such that at least one of R₄ and R₅ is substituted with a nitrogen-containing heterocycle or an amine; or
(ii) taken together to form a 4- to 10-membered heterocycloalkyl that is substituted with from 0 to 4 substituents independently chosen from C₁-C₆alkyl, C₂-C₆alkenyl, C₂-C₆alkynyl, C₁-C₆haloalkyl, mono- or di-(C₁-C₆alkyl)aminoC₀-C₂alkyl, (C₃-C₈cycloalkyl)C₀-C₂alkyl, phenylC₀-C₂alkyl (4- to 8-membered heterocycloalkyl)C₀-C₂alkyl and groups that are taken together to form a C₁-C₃alkylene bridge; each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C6alkyl)aminocarbonyl, C₃-C₇cycloalkyl and 4- to 7-membered heterocycloalkyl.

Within certain aspects, piperazinyl oxoalkyl tetrahydro-beta-carbolines and related analogues provided herein are H3 receptor modulators that exhibit a Kᵢ at a histamine H3 receptor, preferably a human H3 receptor, that is no greater than 4 micromolar, 1 micromolar, 500 nanomolar, 100 nanomolar, 50 nanomolar or 10 nanomolar, as determined using an assay for H3 receptor GTP binding.

Within certain aspects, piperazinyl oxoalkyl tetrahydro-beta-carbolines and related analogues provided herein are labeled with a detectable marker (e.g., radiolabeled or fluorescein conjugated).

The present invention further provides, within other aspects, pharmaceutical compositions comprising at least one piperazinyl oxoalkyl tetrahydro-beta-carboline or related analogue as provided herein in combination with a physiologically acceptable carrier or excipient.

Within further aspects, methods are provided for modulating H3 receptor activity, comprising contacting a cell (e.g., neuronal) expressing H3 receptor with at least one H3 receptor modulator as described herein. Such contact may occur in vivo or in vitro and is generally performed using a concentration of compound that is sufficient to alter H3 receptor GTP binding in vitro (e.g., using the assay provided in Example 7, herein).

The present invention further provides methods for treating a condition responsive to H3 receptor modulation in a patient, comprising administering to the patient a therapeutically effective amount of at least one H3 receptor modulator. Such conditions include, for example, attention deficit disorder, attention deficit hyperactivity disorder, dementia, schizophrenia, cognitive disorders (including mild cognitive impairment), epilepsy, migraine, excessive daytime sleepiness (EDS) and related disorders such as shift work disorder, fatigue and fatigue-related disorders, jet lag, narcolepsy, sleep apnea, allergic rhinitis, vertigo, motion sickness, memory disorders such as Alzheimer's disease, Parkinson's disease, obesity, eating disorders and diabetes.

Within further aspects, the present invention provides methods for determining the presence or absence of H3 receptor in a sample, comprising: (a) contacting a sample with a H3 receptor modulator as described herein under conditions that permit binding of the H3 receptor modulator to H3 receptor; and (b) detecting a level of the H3 modulator bound to H3 receptor.

The present invention also provides packaged pharmaceutical preparations, comprising: (a) a pharmaceutical composition as described herein in a container; and (b) instructions for using the composition to treat one or more conditions responsive to H3 receptor modulation, such as the conditions recited herein.

In yet another aspect, the present invention provides methods of preparing the compounds disclosed herein, including the intermediates.

These and other aspects of the present invention will become apparent upon reference to the following detailed description.

### DETAILED DESCRIPTION

As noted above, the present invention provides piperazinyl oxoalkyl tetrahydro-beta-carbolines and related analogues. Such compounds may be used in vitro or in vivo, to modulate H3 receptor activity in a variety of contexts.

### TERMINOLOGY

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that includes variables (e.g., R₁, Z, etc.). Unless otherwise specified, each variable within such a formula is defined independently of any other variable, and any variable that occurs more than one time in a formula is defined independently at each occurrence.

The phrase “piperazinyl oxoalkyl tetrahydro-beta-carbolines and related analogues,” as used herein, encompasses all compounds of Formula I, including any enantiomers, racemates and stereoisomers, as well as pharmaceutically acceptable salts, solvates (e.g., hydrates) and esters of such compounds.

A “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutically acceptable anions for use in salt formation include, but are not limited to, acetate, 2-acetoxybenzoate, ascorbate, benzoate, bicarbonate, bromide, calcium edetate, carbonate, chloride, citrate, dihydrochloride, diphosphate, ditartrate, edetate, estolate (ethylsuccinate), formate, fumarate, gluceptate, gluconate, glutamate, glycolate, glycollylarsanilate, hexylresorcinate, hydrabamine, hydrobromide, hydrochloride, hydroiodide, hydroxymaleate, hydroxynaphthoate, iodide, isethionate, lactate, lactobionate, malate, maleate, mandelate, methylbromide, methylnitrate, methylsulfate, mucate, napsylate, nitrate, pamoate, pantothenate, phenylacetate, phosphate, polygalacturonate, propionate, salicylate, stearate, subacetate, succinate, sulfamate, sulfanilate, sulfate, sulfonates including besylate (benzenesulfonate), camsylate (camphorsulfonate), edisylate (ethane-1,2-disulfonate), esylate (ethanesulfonate) 2-hydroxyethylsulfonate, mesylate (methanesulfonate), triflate (trifluoromethanesulfonate) and tosylate (p-toluenesulfonate), tannate, tartrate, teoclate and triethiodide. Similarly, pharmaceutically acceptable cations for use in salt formation include, but are not limited to ammonium, benzathine, chloroprocaine, choline, diethanolamine, ethylenediamine, meglumine, procaine, and metals such as aluminum, calcium, lithium, magnesium, potassium, sodium and zinc. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, methanol, isopropanol or acetonitrile, is preferred.

It will be apparent that each compound provided herein may, but need not, be formulated as a solvate (e.g., hydrate), ester or non-covalent complex. In addition, the various crystal forms and polymorphs are within the scope of the present invention. Also provided herein are prodrugs of the compounds of the recited Formulas. A “prodrug” is a compound that may not fully satisfy the structural requirements of the compounds provided herein, but is modified in vivo, following administration to a patient, to produce a compound a formula provided herein. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxy, amino, or sulfhydryl group, respectively. Examples of prodrugs include, but are not limited to, esters such as acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

As used herein, the term “alkyl” refers to a straight or branched chain saturated aliphatic hydrocarbon. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl and 3-methylpentyl.

The term “alkylene” refers to a divalent alkyl group, which may be straight or branched. C₁-C₄alkylene is an alkylene group having from 1 to 4 carbon atoms. “C₀-C₄alkyl” or “C₀-C₄alkylene” is a single covalent bond (C₀) or an alkylene group having from 1 to 4 carbon atoms.

“Alkenyl” refers to straight or branched chain alkene groups, which comprise at least one unsaturated carbon-carbon double bond. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

A “cycloalkyl” is a group that comprises one or more saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Cycloalkyl groups do not comprise an aromatic ring or a heterocyclic ring. A “(C₃-C₈cycloalkyl)C₀-C₂alkyl” is a C₃-C₈cycloalkyl group linked via a single covalent bond or a methylene or ethylene group; C₃-C₇cycloalkyl is a directly linked 3- to 7-membered cycloalkyl.

By “alkoxy,” as used herein, is meant an alkyl group attached via an oxygen bridge. “C₁-C₆alkoxy” has from 1 to 6 carbon atoms in the alkyl portion of the group. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups. Similarly, “alkylthio” refers to an alkyl group attached via a sulfur bridge.

The term “oxo” is used herein to refer to an oxygen substituent of a carbon atom that results in the formation of a carbonyl group (C═O). An oxo group that is a substituent of a nonaromatic carbon atom results in a conversion of —CH₂— to —C(═O)—. An oxo group that is a substituent of an aromatic carbon atom results in a conversion of —CH— to —C(═O)— and may result in a loss of aromaticity.

The term “alkanoyl” refers to an acyl group (e.g., —(C═O)-alkyl), in which carbon atoms are in a linear or branched alkyl arrangement and where attachment is through the carbon of the keto group. Alkanoyl groups have the indicated number of carbon atoms, with the carbon of the keto group being included in the numbered carbon atoms. For example a C₂alkanoyl group is an acetyl group having the formula —(C═O)CH₃; “C₁alkanoyl” refers to —(C═O)H. “C₁-C₆alkanoyl groups” contain from 1 to 6 carbon atoms.

An “alkanone” is a ketone group in which carbon atoms are in a linear or branched alkyl arrangement. “C₃-C₆alkanone” refers to an alkanone having from 3 to 6 carbon atoms, respectively. By way of example, a C₃ alkanone group has the structure —CH₂—(C═O)—CH₃.

Similarly, “alkyl ether” refers to a linear or branched ether substituent (i.e., an alkyl group that is substituted with an alkoxy group). A C₂ alkyl ether has the structure−CH₂—O—CH₃; A C₂-C₆alkyl ether has a total of 2, 3, 4, 5 or 6 carbon atoms.

The term “alkoxycarbonyl” refers to an alkoxy group attached through a keto (—(C═O)—) bridge (i.e., an alkoxycarbonyl group has the general structure —C(═O)—O-alkyl). “C₁alkoxycarbonyl” refers to —C(═O)—O—CH₃; C₃alkoxycarbonyl indicates —C(═O)—O—(CH₂)₂CH₃ or —C(═O)—O—(CH)(CH₃)₂ (i.e., the carbon of the keto bridge is not included in the indicated number of carbon atoms). “C₁-C₆alkoxycarbonyl” groups have from 1 to 6 carbon atoms in the alkyl portion of the group.

“Alkylsulfonyl” refers to groups of the formula —(SO₂)-alkyl, in which the sulfur atom is the point of attachment. “C₁-C₆alkylsulfonyl” has from 1 to 6 carbon atoms in the alkyl group.

The term “aminocarbonyl” refers to an amide group (i.e., —(C═O)NH₂). The term “mono- or di-(C₁-C₆alkyl)aminocarbonyl” refers to groups of the formula —(C═O)—N(R)₂, in which the carbonyl is the point of attachment, one R is C₁-C₆alkyl and the other R is hydrogen or an independently chosen C₁-C₆alkyl.

“Alkylamino” refers to a secondary or tertiary amine that has the general structure —NH-alkyl or —N(alkyl)(alkyl), wherein each alkyl is selected independently from alkyl, cycloalkyl and (cycloalkyl)alkyl groups. Such groups include, for example, mono- and di-(C₁-C₆alkyl)amino groups, in which each C₁-C₆alkyl may be the same or different.

“Alkylaminoalkyl” refers to an alkylamino group linked via an alkylene group (i.e., a group having the general structure -alkylene-NH-alkyl or -alkylene-N(alkyl)(alkyl)) in which each alkyl is selected independently from alkyl, cycloalkyl and (cycloalkyl)alkyl groups. Alkylaminoalkyl groups include, for example, mono- and di-(C₁-C₈alkyl)aminoC₁-C₈alkyl, mono- and di-(C₁-C₆alkyl)aminoC₁-C₆alkyl and mono- and di-(C₁-C₆alkyl)aminoC₁-C₄alkyl. “Mono- or di-(C₁-C₆alkyl)aminoC₀-C₂alkyl” refers to a mono- or di-(C₁-C₆alkyl)amino group linked via a single covalent bond or a methylene or ethylene group. The following are representative alkylaminoalkyl groups:

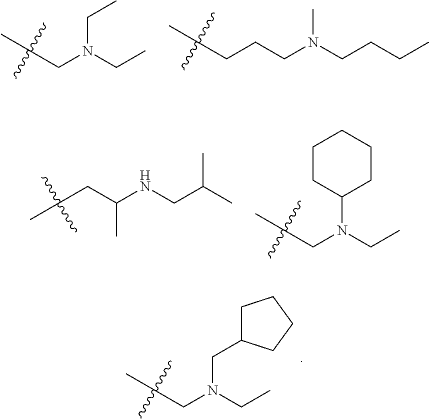

It will be apparent that the definition of “alkyl” as used in the terms “alkylamino” and “alkylaminoalkyl” differs from the definition of “alkyl” used for all other alkyl-containing groups, in the inclusion of cycloalkyl and (cycloalkyl)alkyl groups (e.g., (C₃-C₇cycloalkyl)C₀-C₆alkyl).

The term “halogen” refers to fluorine, chlorine, bromine or iodine.

A “haloalkyl” is an alkyl group that is substituted with 1 or more halogen atoms (e.g., “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “C₁-C₆haloalkoxy” groups have 1 to 6 carbon atoms.

A dash (“-”) that is not between two letters or symbols is used to indicate a point of attachment for a substituent. For example, —CONH₂ is attached through the carbon atom.

A “carbocycle” or “carbocyclic group” comprises at least one ring formed entirely by carbon-carbon bonds (referred to herein as a carbocyclic ring), and does not contain a heterocycle. Certain representative carbocycles are cycloalkyl as described above. Other carbocycles are aryl (i.e., contain at least one aromatic ring). PhenylC₀-C₂alkyl is a phenyl, benzyl or phenethyl moiety.

A “heterocycle” or “heterocyclic group” has from 1 to 3 fused, pendant or spiro rings (and typically from 3 to 15 ring members in total), at least one of which is a heterocyclic ring (i.e., one or more ring atoms is a heteroatom independently chosen from O, S and N, with the remaining ring atoms being carbon). Additional rings, if present, may be heterocyclic or carbocyclic. Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and certain heterocycles comprising fused, pendant or spiro rings contain from 9 to 14 ring members. Certain heterocycles comprise a sulfur atom as a ring member; in certain embodiments, the sulfur atom is oxidized to SO or SO₂. Heterocycles may be optionally substituted with a variety of substituents, as indicated. Unless otherwise specified, a heterocycle may be a heterocycloalkyl group (i.e., each ring is saturated or partially saturated) or a heteroaryl group (i.e., at least one ring within the group is aromatic), and may be linked via any ring atom, provided that a stable compound results.

Heterocyclic groups include, for example, acridinyl, azepanyl, azocinyl, benzimidazolyl, benzimidazolinyl, benzisothiazolyl, benzisoxazolyl, benzofuranyl, benzothiofuranyl, benzothiophenyl, benzoxazolyl, benzothiazolyl, benzotriazolylcarbazolyl, benztetrazolyl, NH-carbazolyl, carbolinyl, chromanyl, chromenyl, cinnolinyl, decahydroquinolinyl, dihydrofuro[2,3-b]tetrahydrofuran, dihydroisoquinolinyl, dihydrotetrahydrofuranyl, 1,4-dioxa-8-aza-spiro[4.5]dec-8-yl, dithiazinyl, furanyl, furazanyl, imidazolinyl, imidazolidinyl, imidazolyl, indazolyl, indolenyl, indolinyl, indolizinyl, indolyl, isobenzofuranyl, isochromanyl, isoindazolyl, isoindolinyl, isoindolyl, isothiazolyl, isoxazolyl, isoquinolinyl, morpholinyl, naphthyridinyl, octahydroisoquinolinyl, oxadiazolyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, piperidinyl, piperidonyl, pteridinyl, purinyl, pyranyl, pyrazinyl, pyrazolidinyl, pyrazolinyl, pyrazolyl, pyridazinyl, pyridoimidazolyl, pyridooxazolyl, pyridothiazolyl, pyridyl, pyrimidyl, pyrrolidinyl, pyrrolidonyl, pyrrolinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienothiazolyl, thienooxazolyl, thienoimidazolyl, thienyl, thiophenyl, thiomorpholinyl and variants thereof in which the sulfur atom is oxidized, triazinyl, xanthenyl and any of the foregoing that are substituted as described herein.

Certain heterocycles are 5- or 6-membered heteroaryl groups (e.g., pyridyl, pyrimidyl and pyridazinyl), each of which may be substituted as indicated. Other heterocycles are 4- to 8-membered heterocycloalkyl groups, which are saturated or partially saturated heterocycles as described above, containing 4, 5, 6, 7 or 8 ring members. A “(4- to 8-membered heterocycloalkyl)C₀-C₂alkyl” is a 4- to 8-membered heterocycloalkyl group that is linked via a single covalent bond or a methylene or ethylene group.

A “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member. The term “substitution” refers to replacing one or more hydrogen atoms in a molecular structure with a substituent as described above, such that the valence on the designated atom is not exceeded, and such that a chemically stable compound (i.e., a compound that can be isolated, characterized, and tested for biological activity) results from the substitution.

Groups that are “optionally substituted” are unsubstituted or substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of permissible substituents. Certain optionally substituted groups are substituted with from 0 to 2, 3 or 4 independently selected substituents (i.e., are unsubstituted or substituted with up to the recited maximum number of substituents). Other optionally substituted groups are substituted with at least one substituent (e.g., substituted with from 1 to 2, 3 or 4 independently selected substituents).

Unless otherwise specified, the term “H3 receptor” is used herein to refer to any histamine H3 subtype receptor, including human H3 receptor (see, e.g., U.S. Pat. No. 6,136,559), H3 receptor found in other mammals and chimeric receptors retaining H3 function, including the chimeric H3 receptor provided as SEQ ID NO:8 in U.S. patent application Ser. No. 11/355,711, which published as US 2006/0188960.

A “H3 receptor modulator,” also referred to herein as a “modulator,” is a compound that modulates H3 receptor GTP binding. A H3 receptor modulator may be a H3 receptor agonist or antagonist. A modulator binds with “high affinity” if the Kᵢ at H3 receptor is less than 4 micromolar, preferably less than 1 micromolar, 500 nanomolar, 100 nanomolar, 50 nanomolar or 10 nanomolar. A representative assay for evaluating an effect on H3 receptor GTP binding is provided in Example 7, herein.

Unless otherwise specified, the terms “IC₅₀” and “EC₅₀,” as used herein, refer to values obtained using the assay as described in Example 7.

A modulator is considered an “antagonist” if it detectably inhibits H3 receptor agonist-stimulated GTP binding (using, for example, the representative assay provided in Example 7); in general, such an antagonist inhibits such GTP binding with a IC₅₀ value of less than 4 micromolar, preferably less than 1 micromolar, 500 nanomolar, 100 nanomolar, 50 nanomolar or 10 nanomolar. H3 receptor antagonists include neutral antagonists and inverse agonists.

An “inverse agonist” of H3 receptor is a compound that reduces the GTP binding activity of H3 receptor below its basal activity level in the absence of added agonist. Inverse agonists of H3 receptor may also inhibit the activity in the presence of agonist. The basal activity of H3 receptor, as well as the reduction in H3 receptor GTP binding activity due to the presence of H3 receptor antagonist, may be determined using the assay of Example 7.

A “neutral antagonist” of H3 receptor is a compound that inhibits the activity of H3 receptor agonist, but does not significantly change the basal activity of the receptor (i.e., within the assay of Example 7 performed in the absence of agonist, H3 receptor activity is reduced by no more than 10%, preferably by no more than 5%, and more preferably by no more than 2%; most preferably, there is no detectable reduction in activity). The basal activity is the level of GTP binding observed in the assay in the absence of added histamine or any other agonist, and in the further absence of any test compound. Neutral antagonists of H3 receptor may, but need not, inhibit the binding of agonist to H3 receptor.

As used herein a “H3 receptor agonist” is a compound that elevates the activity of the receptor above the basal activity level of the receptor. H3 receptor agonist activity may be identified using the representative assay provided in Example 7. In general, such an agonist has an EC₅₀ value of less than 4 micromolar, preferably less than 1 micromolar, 500 nanomolar, 100 nanomolar, 50 nanomolar or 10 nanomolar within the assay provided in Example 7. If the GTP binding activity brought about by a test compound attains the same level to that of histamine, it is defined as a full agonist. If the level of GTP binding activity brought about by a test compound is above baseline but below the level attained by histamine, it is defined as a partial agonist. Preferred antagonists do not elevate GTP binding activity under such conditions more than 10% above baseline, preferably not more than 5% above baseline, and most preferably not more than 2% above baseline.

A “therapeutically effective amount” (or dose) is an amount that, upon administration to a patient, results in a discernible patient benefit (e.g., provides detectable relief from a condition being treated). Such relief may be detected using any appropriate criteria, including alleviation of one or more symptoms characteristic of the condition. A therapeutically effective amount or dose generally results in a concentration of compound in a body fluid (such as blood, plasma, serum, CSF, synovial fluid, lymph, cellular interstitial fluid, tears or urine) that is sufficient to alter H3 receptor GTP binding in vitro. It will be apparent that the discernible patient benefit may be apparent after administration of a single dose, or may become apparent following repeated administration of the therapeutically effective dose according to a predetermined regimen, depending upon the indication for which the compound is administered.

A “patient” is any individual treated with a piperazinyl oxoalkyl tetrahydro-beta-carboline or related analogue provided herein. Patients include humans, as well as other animals such as companion animals (e.g., dogs and cats) and livestock. Patients may be experiencing one or more symptoms of a condition responsive to H3 receptor modulation, or may be free of such symptom(s) (e.g., treatment may be prophylactic).

### Piperazinyl Oxoalkyl Tetrahydro-Beta-Carbolines and Related Analogues

As noted above, the present invention provides piperazinyl oxoalkyl tetrahydro-beta-carbolines and related analogues of Formula I:

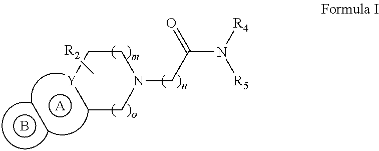

in which variables are as described above.

Within certain aspects, such compounds are H3 receptor modulators that may be used in a variety of contexts, including in the therapeutic treatment of human and animal patients as discussed below. H3 receptor modulators may also be used within in vitro assays (e.g., assays for receptor activity), and as probes for detection and localization of H3 receptor.

Certain piperazinyl oxoalkyl tetrahydro-beta-carbolines and related analogues of Formula I further satisfy Formula Ia:

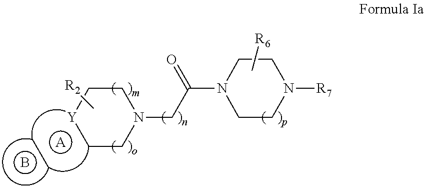

in which:
p is 1, 2 or 3;
R₆ represents from 0 to 4 substituents independently chosen from C₁-C₆alkyl, C₁-C₆haloalkyl and groups that are taken together to form a C₁-C₃alkylene bridge;
R₇ is C₁-C₆alkyl, C₂-C₆alkenyl, C₂-C₆alkynyl, (C₃-C₈cycloalkyl)C₀-C₂alkyl, phenylC₀-C₂alkyl or (4- to 8-membered heterocycloalkyl)C₀-C₂alkyl, each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl; and
   the remaining variables are as described for Formula I.

Other piperazinyl oxoalkyl tetrahydro-beta-carbolines and related analogues of Formula I further satisfy Formula Ib:

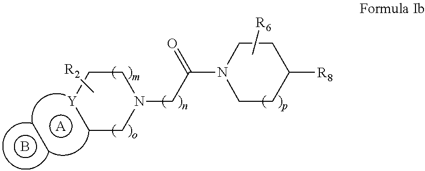

in which:
p is 0, 1, 2 or 3;
R₆ is as described for Formula Ia;
R₈ is mono- or di-(C₁-C₆alkyl)aminoC₀-C₂alkyl or (4- to 8-membered heterocycloalkyl)C₀-C₂alkyl, each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl; and
   the remaining variables are as described for Formula I.

Further piperazinyl oxoalkyl tetrahydro-beta-carbolines and related analogues of Formula I further satisfy Formula Ic or Formula Id:

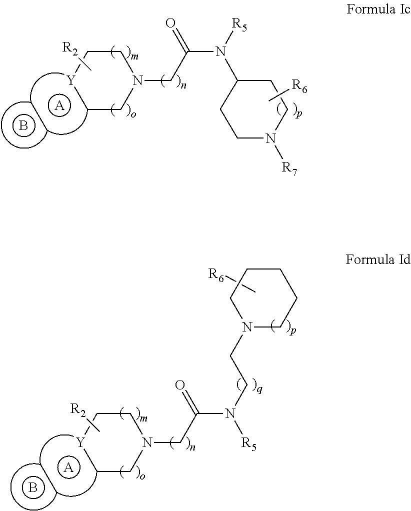

in which p is 0, 1, 2 or 3; R₆ and R₇ are as described for Formula Ia; q is 1, 2 or 3; and the remaining variables are as described for Formula I.

Certain piperazinyl oxoalkyl tetrahydro-beta-carbolines and related analogues of Formula I further satisfy Formula II:

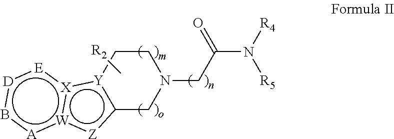

or are a pharmaceutically acceptable salt, solvate or ester thereof.

### Within Formula II:

A, B, D and E are independently N or CR₁; such that

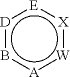

is aromatic;
W, X and Y are independently C or N;
Z is CR₉, N, NR₃, S or O;
n is 0, 1, 2 or 3;
m is 0, 1 or 2;
o is 1 or 2;
Each R₁ is independently:
   (i) hydrogen, amino, halogen, cyano, hydroxy, nitro or oxo; or
   (ii) C₁-C₆alkyl, C₂-C₆alkenyl, C₂-C₆alkynyl, (C₃-C₈cycloalkyl)C₀-C₂alkyl, C₁-C6alkoxy, C₁-C₆haloalkyl, C₂-C₆alkyl ether, C₁-C₆alkylsulfonyl, mono- or di-(C₁-C₆alkyl)aminoC₀-C₄alkyl, phenylC₀-C₂alkyl or (5- to 7-membered heterocycle)C₀-C₂alkyl; each of which is unsubstituted or substituted with oxo, C₁-C₆alkyl or C₁-C₆alkoxy;
R₂ represents from 0 to 4 substituents independently chosen from C₂-C₆alkyl, C₂-C₆alkenyl, C₂-C₆alkynyl, (C₃-C₈cycloalkyl)C₀-C₂alkyl, C₁-C₆haloalkyl and groups that are taken together to form a C₁-C₃alkylene bridge;
R₃ is hydrogen, C₁-C₆alkyl, C₂-C₆alkenyl, C₂-C₆alkynyl, C₁-C₆haloalkyl, C₂-C₆alkyl ether, C₂-C₆-aminoalkyl, or mono- or di-(C₁-C₆alkyl)aminoC₂-C₆alkyl;
R₄ and R₅ are:
(i) independently chosen from C₁-C₆alkyl, C₂-C₆alkenyl, C₂-C₆alkynyl, C₁-C₆haloalkyl and C₂-C₆alkyl ether; each of which is substituted with from 0 to 4 substituents independently chosen from amino, cyano, oxo, mono- or di-(C₁-C₆alkyl)amino, mono- or di-(C₁-C₆alkyl)aminocarbonyl, and 5- to 7-membered heterocycloalkyl; such that at least one of R₄ and R₅ is substituted with a nitrogen-containing heterocycle or an amine; or
(ii) taken together to form a 4- to 10-membered heterocycloalkyl that is substituted with from 0 to 4 substituents independently chosen from C₁-C₆alkyl, C₂-C₆alkenyl, C₂-C₆alkynyl, C₁-C₆haloalkyl, mono- or di-(C₁-C₆alkyl)aminoC₀-C₂alkyl, (C₃-C₈cycloalkyl)C₀-C₂alkyl, phenylC₀-C₂alkyl, (4- to 7-membered heterocycloalkyl)C₀-C₂alkyl and groups that are taken together to form a C₁-C₃alkylene bridge; each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl; and
R₉ is hydrogen, amino, C₁-C₆alkyl, C₂-C₆alkenyl, C₂-C₆alkynyl, C₁-C₆haloalkyl, C₂-C₆alkyl ether, C₁-C₆-aminoalkyl, or mono- or di-(C₁-C₆alkyl)aminoC₀-C₆alkyl.

Certain piperazinyl oxoalkyl tetrahydro-beta-carbolines and related analogues of Formula II further satisfy Formula IIa:

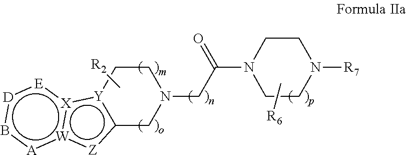

in which p, R₆ and R₇ are as described for Formula Ia; and the remaining variables are as described for Formula II.

Other piperazinyl oxoalkyl tetrahydro-beta-carbolines and related analogues of Formula II further satisfy Formula IIb:

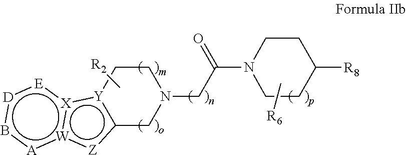

in which p, R₆ and R₈ are as described for Formula Ib; and the remaining variables are as described for Formula II.

Further piperazinyl oxoalkyl tetrahydro-beta-carbolines and related analogues of Formula II further satisfy Formula IIc or Formula IId:

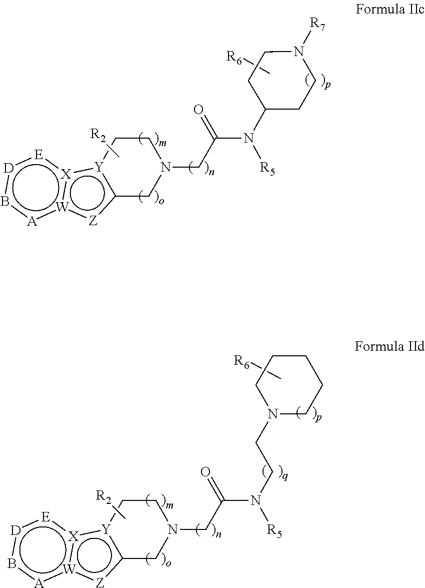

in which p, R₆ and R₇ are as described for Formula IIa; q is 1, 2 or 3; and the remaining variables are as described for Formula II.

Certain piperazinyl oxoalkyl tetrahydro-beta-carbolines and related analogues of Formula I, Formula II and the various subformulas thereof further satisfy one or more of the following:

(i) W and Y are carbon, and Z is oxygen or sulfur.

(ii) X, W and Y are carbon, and Z is nitrogen or NR₃.

(iii) X and W are carbon, Y is nitrogen, and Z is nitrogen or NR₃.

(iv) X and W are carbon; Y is nitrogen; and Z is CR₃.

(v) W is carbon, and exactly one of A, B, D and E is nitrogen.

(vi) W is carbon, A is nitrogen, and exactly one of B, D and E is nitrogen.

(vii) A, B, D and E are independently chosen from CR₁.

(viii) A is carbon; and exactly two of B, D and E are nitrogen.

Still further piperazinyl oxoalkyl tetrahydro-beta-carbolines and related analogues of Formula II further satisfy one of the following Formulas, in which variables are generally as described above:

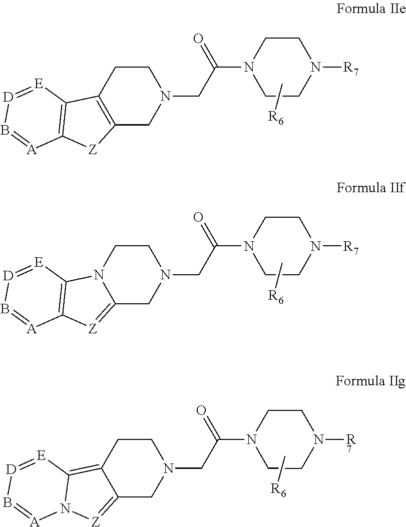

Within certain embodiments of Formulas I, II and subformulas thereof:
A, B, D and E (B, D and E in the case of Formula IIg) are independently nitrogen or CR₁; such that exactly zero, one or two of A, B, D and E are nitrogen;
R₆ represents from 0 to 2 substituents independently chosen from C₁-C₆alkyl and C₁-C₆haloalkyl; and
R₇ is cyclopropyl, cyclobutyl, cyclopentyl, cyclohexyl or C₃-C₆alkyl.

Within certain embodiments of Formulas I, II and IIa-IIe, Z is oxygen, sulfur or NR₃. Within certain embodiments of Formulas I, II, IIa-IId, IIf and IIg, Z is nitrogen or CR₃.

Within certain embodiments of Formulas I, II and subformulas thereof each R₁ is independently chosen from hydrogen, halogen, cyano, C₁-C₆alkyl, C₁-C₆haloalkyl, (C₃-C₈cycloalkyl)C₀-C₂alkyl, C₂-C₆alkyl ether, C₁-C₆alkoxy, C₁-C₆alkanoyl, mono- or di-(C₁-C₆alkyl)amino and mono- or di-(C₁-C₆alkyl)aminocarbonyl.

Certain piperazinyl oxoalkyl tetrahydro-beta-carbolines and related analogues of Formula I further satisfy Formula III:

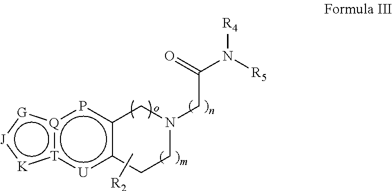

or are a pharmaceutically acceptable salt, solvate or ester thereof. Within Formula III:
G, J and K are independently nitrogen, oxygen, sulfur or CR₁;
Q and T are independently carbon or nitrogen;
P and U are independently CR₃ or nitrogen;
n is 0, 1, 2 or 3;
m is 0, 1 or 2;
o is 1 or 2;
Each R₁ is independently:
   (i) hydrogen, amino, halogen, cyano, hydroxy, nitro or oxo; or
   (ii) C₁-C₆alkyl, C₂-C₆alkenyl, C₂-C₆alkynyl, (C₃-C₈cycloalkyl)C₀-C₂alkyl, C₁-C₆alkoxy, C₁-C₆haloalkyl, C₂-C₆alkyl ether, C₁-C₆alkylsulfonyl, mono- or di-(C₁-C₆alkyl)aminoC₀-C₄alkyl, phenylC₀-C₂alkyl or (5- to 7-membered heterocycle)C₀-C₂alkyl; each of which is unsubstituted or substituted with oxo, C₁-C₆alkyl, or C₁-C₆alkoxy;
R₂ represents from 0 to 4 substituents independently chosen from C₁-C₆alkyl, C₂-C₆alkenyl, C₂-C₆alkynyl, (C₃-C₈cycloalkyl)C₀-C₂alkyl, C₁-C₆haloalkyl and groups that are taken together to form a C₁-C₃alkylene bridge;
R₃ is hydrogen, C₁-C₆alkyl, C₂-C₆alkenyl, C₂-C₆alkynyl, C₁-C₆haloalkyl, C₂-C₆alkyl ether, C₂-C₆-aminoalkyl, or mono- or di-(C₁-C₆alkyl)aminoC₂-C₆alkyl;
R₄ and R₅ are:
(i) independently chosen from C₁-C₆alkyl, C₂-C₆alkenyl, C₂-C₆alkynyl, C₁-C₆haloalkyl and C₂-C₆alkyl ether; each of which is substituted with from 0 to 4 substituents independently chosen from amino, cyano, oxo, mono- or di-(C₁-C₆alkyl)amino, mono- or di-(C₁-C₆alkyl)aminocarbonyl, and 5- to 7-membered heterocycloalkyl; such that at least one of R₄ and R₅ is substituted with a nitrogen-containing heterocycle or an amine; or
(ii) taken together to form a 4- to 10-membered heterocycloalkyl that is substituted with from 0 to 4 substituents independently chosen from C₁-C₆alkyl, C₂-C₆alkenyl, C₂-C₆alkynyl, C₁-C₆haloalkyl, mono- or di-(C₁-C₆alkyl)aminoC₀-C₂alkyl, (C₃-C₈cycloalkyl)C₀-C₂alkyl, phenylC₀-C₂alkyl, (4- to 7-membered heterocycloalkyl)C₀-C₂alkyl and groups that are taken together to form a C₁-C₃alkylene bridge; each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl.

Certain piperazinyl oxoalkyl tetrahydro-beta-carbolines and related analogues of Formula III further satisfy Formula IIIa:

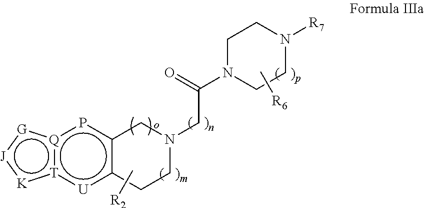

in which p is 1, 2 or 3, and R₆ and R₇ are as described for Formula Ia.

Other piperazinyl oxoalkyl tetrahydro-beta-carbolines and related analogues of Formula III further satisfy Formula IIIb:

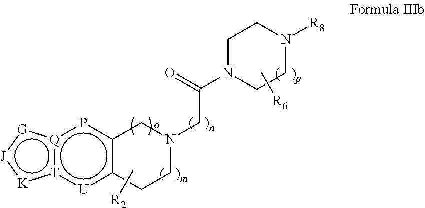

in which p is 0, 1, 2 or 3; and R₆ and R₈ are as described for Formula Ib.

Further piperazinyl oxoalkyl tetrahydro-beta-carbolines and related analogues of Formula III satisfy Formula IIIc or Formula IIId:

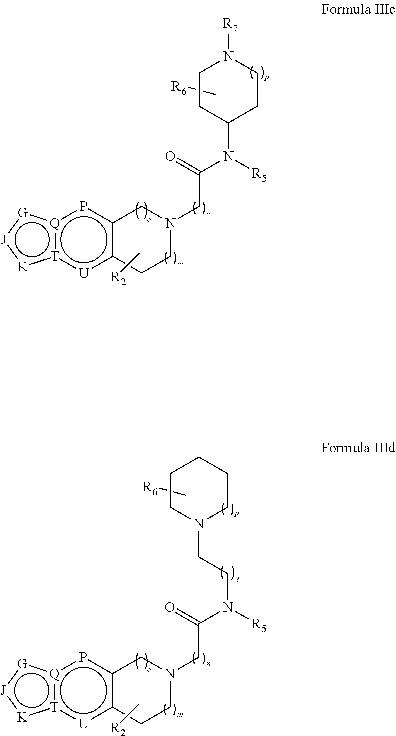

in which p is 0, 1, 2 or 3; and R₆ and R₇ are as described for Formula IIIa.

Within certain compounds of Formula III and the subformulas thereof, one or more of the following conditions is met:

(a) Q and U are nitrogen; T is carbon; and P is CH.

(b) P and T are nitrogen; Q is carbon; and U is CH.

(c) G is nitrogen; and J and K are CH.

(d) K is nitrogen; and J and G are CH.

Still further piperazinyl oxoalkyl tetrahydro-beta-carbolines and related analogues of Formula III satisfy Formula IIIe or Formula IIIf:

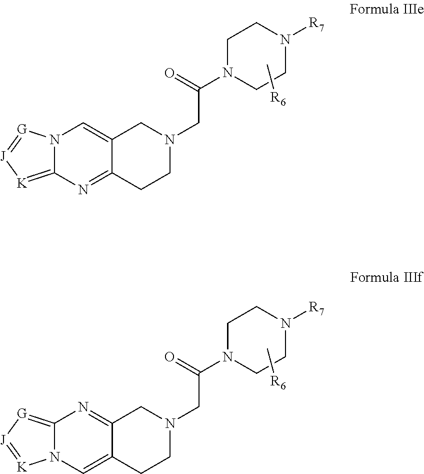

wherein G, J and K are independently nitrogen or CR₁; such that exactly zero, one or two of G, J and K are nitrogen; R₆ represents from 0 to 2 substituents independently chosen from C₁-C₆alkyl and C₁-C₆haloalkyl; and R₇ is cyclopropyl, cyclobutyl, cyclopentyl, cyclohexyl or C₃-C₆alkyl.

Within certain compounds of Formula I, II or III:

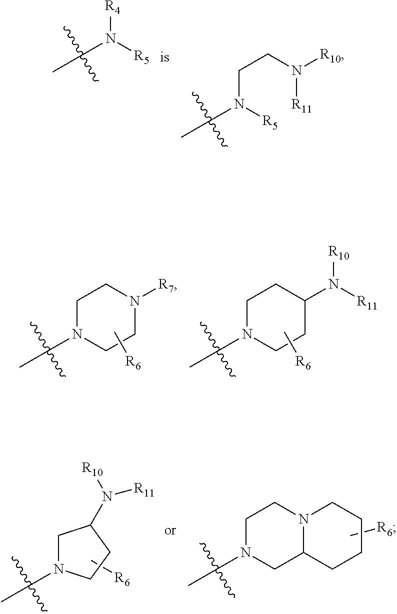

in which R₅, R₆ and R₇ are as described above and R₁₀ and R₁₁ are independently chosen from: (i) hydrogen; and (ii) C₁-C₆alkyl, C₂-C₆alkenyl, C₂-C₆alkynyl, (C₃-C₈cycloalkyl)C₀-C₂alkyl, (4- to 7-membered heterocycloalkyl)C₀-C₂alkyl, and groups that are taken together to form a 4- to 7-membered heterocycloalkyl, each of which is optionally substituted with any group consistent with Formula I. In certain such compounds, R₆ represents from 0 to 2 substituents independently chosen from C₁-C₆alkyl and C₁-C₆haloalkyl; and R₇ is cyclopropyl, cyclobutyl, cyclopentyl, cyclohexyl or C₃-C₆alkyl; in further such compounds, R₅ is hydrogen or C₁-C₆alkyl; and R₁₀ and R₁₁ are independently chosen from: (i) hydrogen; and (ii) C₁-C₆alkyl, C₂-C₆alkenyl, C₂-C₆alkynyl, (C₃-C₈cycloalkyl)C₀-C₂alkyl, and groups that are taken together to form a 4- to 7-membered heterocycloalkyl, each of which is substituted with from 0 to 4 substituents independently chosen from oxo, amino, cyano, C₁-C₄alkyl mono- or di-(C₁-C₄alkyl)amino.

Representative piperazinyl oxoalkyl tetrahydro-beta-carbolines and related analogues provided herein include, but are not limited to, those specifically described in Examples 1-3. It will be apparent that the specific compounds recited herein are representative only, and are not intended to limit the scope of the present invention. Further, as noted above, all compounds of the present invention may be present as a free acid or base or as a pharmaceutically acceptable salt, solvate or ester.

In certain aspects, piperazinyl oxoalkyl tetrahydro-beta-carbolines and related analogues provided herein are H3 receptor modulators, as determined using an assay for H3 receptor GTP binding. References herein to an “assay for H3 receptor GTP binding” are intended to refer to the in vitro GTP binding assay provided in Example 7, which may be performed in the presence or absence of added agonist. Briefly, to assess H3 receptor agonist-stimulated GTP binding, a H3 receptor preparation is incubated with a H3 receptor agonist (e.g., histamine or an analogue thereof such as R-alpha-methyhistamine), labeled (e.g., ³⁵S) GTP and unlabeled test compound. Within the assays provided herein, the H3 receptor used is preferably mammalian H3 receptor (e.g., human or rat H3 receptor, and preferably human H3 receptor), and more preferably a chimeric human H3 receptor such as a receptor having the sequence provided in SEQ ID NO:8. The H3 receptor may be recombinantly expressed or naturally expressed. The H3 receptor preparation may be, for example, a membrane preparation from cells that recombinantly express H3 receptor. Incubation with a H3 receptor modulator results in a decrease or increase in the amount of label bound to the H3 receptor preparation, relative to the amount of label bound in the absence of the compound.

As noted above, piperazinyl oxoalkyl tetrahydro-beta-carbolines and related analogues that are H3 receptor antagonists are preferred within certain embodiments. When agonist-contacted cells are contacted with a piperazinyl oxoalkyl tetrahydro-beta-carboline or related analogue that is a H3 receptor antagonist, the response is preferably reduced by at least 20%, more preferably at least 50% and still more preferably at least 80%, as compared to cells that are contacted with the agonist in the absence of the piperazinyl oxoalkyl tetrahydro-beta-carboline or related analogue. The IC₅₀ for H3 receptor antagonists provided herein is preferably less than 4 micromolar, less than 1 micromolar, less than 500 nM, less than 100 nM, less than 50 nM or less than 10 nM. In certain embodiments, H3 receptor antagonists provided herein exhibit no detectable agonist activity in the assay of Example 7 at a concentration of compound equal to the IC₅₀. Certain preferred antagonists exhibit no detectable agonist activity in the assay at a concentration of the antagonist that is 100-fold higher than the IC₅₀.

In certain embodiments, preferred H3 receptor modulators provided herein are non-sedating. In other words, a dose of H3 receptor modulator that is twice the minimum therapeutically effective dose causes only transient (i.e., lasting for no more than ½ the time that the therapeutic effect lasts) or preferably no statistically significant sedation in an animal model assay of sedation (using the method described by Fitzgerald et al. (1988) Toxicology 49(2-3):433-9). Preferably, a dose that is any of 5, 10, 20, 30, 40, 50, 60, 70, 80, 90 or 100 times the minimum therapeutically effective dose does not produce statistically significant sedation. More preferably, a H3 receptor modulator does not produce sedation at oral doses of less than 140 mg/kg (preferably less than 50 mg/kg, more preferably less than 30 mg/kg).

If desired, H3 receptor modulators provided herein may be evaluated for certain pharmacological properties including, but not limited to, oral bioavailability (preferred compounds are orally bioavailable to an extent allowing for therapeutically effective concentrations of the compound at oral doses of less than 140 mg/kg, preferably less than 50 mg/kg, more preferably less than 30 mg/kg, even more preferably less than 10 mg/kg, and still more preferably less than 1 mg/kg), toxicity (a preferred H3 receptor modulator is nontoxic when a therapeutically effective amount is administered to a subject), side effects (a preferred H3 receptor modulator produces side effects comparable to placebo when a therapeutically effective amount of the compound is administered to a subject), serum protein binding and in vitro and in vivo half-life (a preferred H3 receptor modulator exhibits an in vivo half-life allowing for Q.I.D. dosing, preferably T.I.D. dosing, more preferably B.I.D. dosing, and most preferably once-a-day dosing). In addition, differential penetration of the blood brain barrier may be desirable for certain H3 receptor modulators. Routine assays that are well known in the art may be used to assess these properties, and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays or whole serum binding assays. In vitro half-lives of compounds may be predicted from assays of microsomal half-life as described within Example 8 of PCT Publication Number WO 06/089076.

As noted above, preferred piperazinyl oxoalkyl tetrahydro-beta-carbolines and related analogues are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, or (4) does not cause substantial release of liver enzymes.

As used herein, a compound that does not substantially inhibit cellular ATP production is a compound that satisfies the criteria set forth in Example 9 of PCT Publication Number WO 06/089076. In other words, cells treated as described in Example 9 therein with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more highly preferred embodiments, such doses do not elevate such serum levels of ALT, LDH or AST by more than 75% or 50% over matched controls. Alternatively, a H3 receptor modulator does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the EC₅₀ or IC₅₀ for the compound.

In other embodiments, certain preferred compounds do not substantially inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred H3 receptor modulators do not induce sister chromatid exchange (e.g., in Chinese hamster ovary cells) at such concentrations.

For detection purposes, as discussed in more detail below, piperazinyl oxoalkyl tetrahydro-beta-carbolines and related analogues provided herein may be isotopically-labeled or radiolabeled. For example, one or more atoms may be replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

### Preparation of Piperazinyl Oxoalkyl Tetrahydro-Beta-Carbolines and Related Analogues

Piperazinyl oxoalkyl tetrahydro-beta-carbolines and related analogues provided herein may generally be prepared using standard synthetic methods. Starting materials illustrated in the schemes and in the examples are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon as appreciated by those skilled in the art. Each variable in the following schemes refers to any group consistent with the description of the piperazinyl oxoalkyl tetrahydro-beta-carbolines and related analogues provided herein.

Certain abbreviations used in the following Schemes and elsewhere herein are:

BOC tert-butyl carboxyl
BOP benzotriazol-1-yl-oxy-tris(dimethylamino)phosphonium hexafluorophosphate
Bu butyl
CDCl₃ deuterated chloroform
δ chemical shift
DCM dichloromethane
DMA dimethyl acetal
DMC 2-chloro-1,3-dimethylimidazolinium chloride
DMF dimethylformamide
dppf 1,1′-Bis(diphenylphosphino)ferrocene
EtOAc ethyl acetate
Et ethyl
EtOH ethanol
Eq. equivalent(s)
¹H NMR proton nuclear magnetic resonance
HPLC high pressure liquid chromatography
h hour(s)
Hz hertz
LCMS liquid chromatography/mass spectrometry
MS mass spectrometry
(M+1) mass+1
Me methyl
MeOH methanol
MsCl methanesulfonyl chloride
min minute(s)
Pd₂(dba)₃ tris(dibenzylideneacetone)dipalladium(0)
Pd(PPh₃)₄ tetrakis(triphenylphosphine)palladium(0)
PG protecting group, such as BOC or a benzyl group
PPh₃ triphenylphosphine
PTLC preparative thin layer chromatography
rt room temperature
TEA triethylamine
TfO trifluoromethanesulfonyloxy
Tf₂O trifluoromethanesulfonic anhydride
THF tetrahydrofuran
Xantphos 9,9-dimethyl-4,5-bis(diphenylphosphino)xanthene

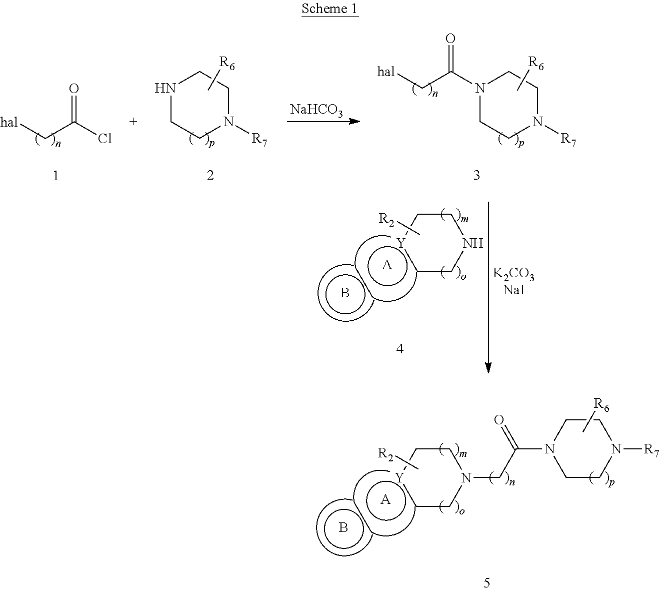

Scheme 1 illustrates the preparation of compounds 5. Cyclic amine 2 is reacted with halogen substituted acylchloride 1 in the presence of a base such as sodium bicarbonate to afford halogen substituted carboxamide 3, which is treated with tetrahydro-1H-beta-carbolines or related amine analogue 4 in the presence of a base such as potassium carbonate to produce 5.

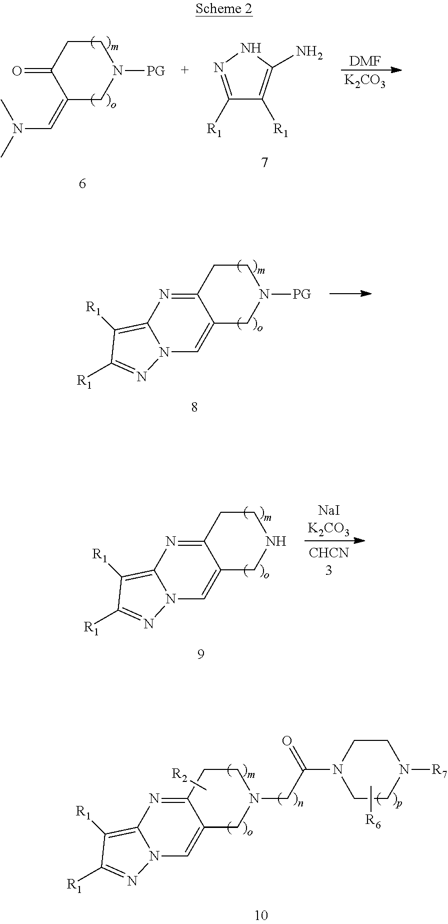

Scheme 2 illustrates the preparation of compounds 10. Condensation of 6 with 5-aminopyrazole 7 provides 8, which is then converted to amine 9 upon deprotection. Alkylation of 9 with carboxamide 3 under standard alkylation conditions provides 10.

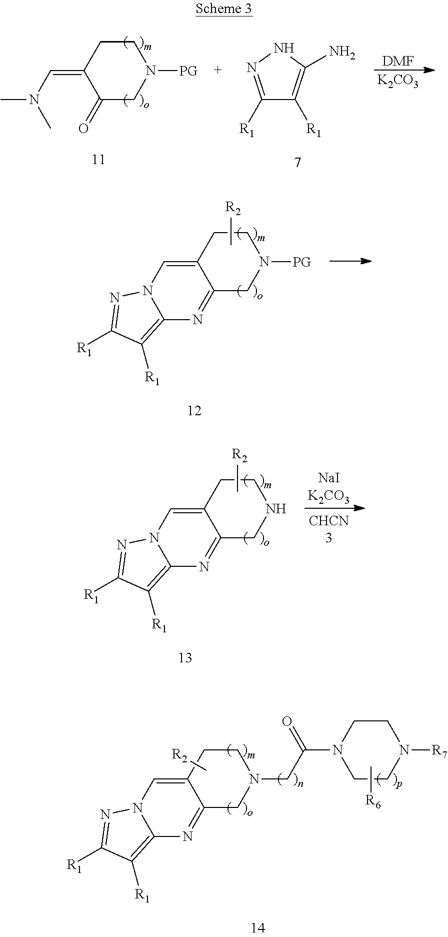

Scheme 3 illustrates the preparation of compounds 14. Condensation of 11 with 5-aminopyrazole 7 provides 12, which is then converted to amine 13 upon deprotection. Alkylation of 13 with carboxamide 3 under standard alkylation conditions provides 14.

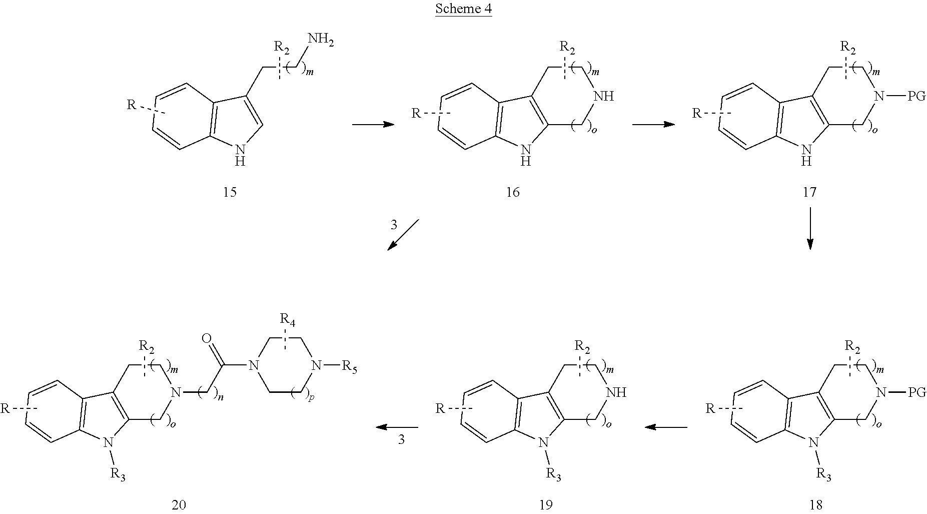

Compounds 20 are prepared in accordance with Scheme 4, in which “R” represents from 0 to 4 ring substituents indicated as R₁ in certain Formulas herein. Starting from any of a variety of commercially available tryptamines 15, 2,3,4,9-tetrahydro-1H-beta-carboline 16 is prepared using established literature procedures (e.g., Organic Syntheses (1971) 51:136-38; and Journal of Labeled Compounds & Radiopharmaceuticals (2005) 48(5):323-30). Alternatively, the synthesis may be started with a commercially available 2,3,4,9-tetrahydro-1H-beta-carboline 16. Direct alkylation of 16 with carboxamide 3 forms 20. Protection of secondary amine forms 17, which can be alkylated directly by an alkylhalide or arylated by a Buchwald reaction to form 18. Deprotection of 18 forms 19 and alkylation with carboxamide 3 under standard alkylation conditions provides 20.

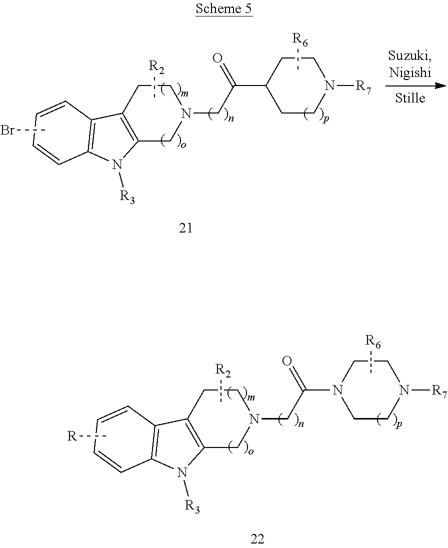

Scheme 5 illustrates the preparation of biaryl (R=aryl or heteroaryl) and cyano (R═CN) analogues 22, in which “R” represents from 0 to 4 ring substituents indicated as R₁ in certain Formulas. The appropriately substituted bromo-2,3,4,9-tetrahydro-1H-beta-carboline 21 is converted to 22 (R=aryl or heteroaryl) by palladium-catalyzed coupling, such as Suzuki coupling, Nigishi coupling or Stille coupling. Cyano-analogue 22 (R═CN) is made by a Nigishi reaction using Zn(CN)₂.

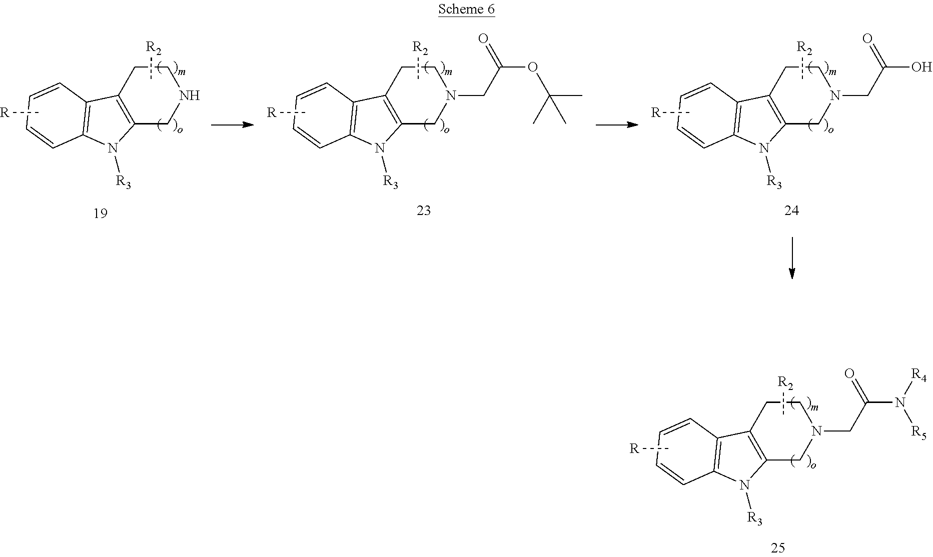

Scheme 6 illustrates the preparation of amide analogues 25. Compound 19 is alkylated with t-butyl bromoacetate to form 23. After refluxing in 4N HCl dioxane, deprotection of the t-butyl ester forms carboxylic acid 24, which is transformed into amide 25 upon coupling with an appropriate amine in the presence of a coupling reagent such as DMC or BOP.

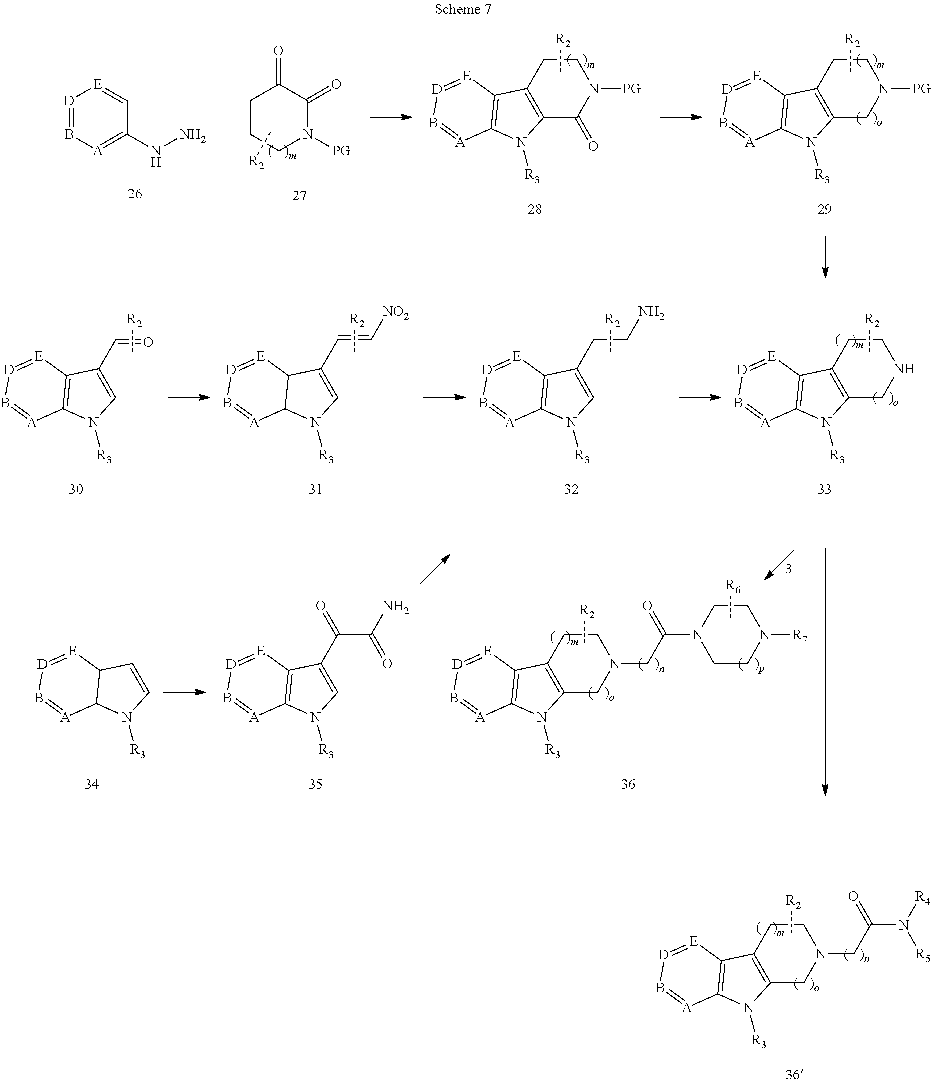

Scheme 7 illustrates the preparation of azaindole analogues 36 and 36′. Compound 28 is formed by a Fisher indole reaction between 26 and 27 (Annali di Chimica (Rome, Italy) (1965) 55(12):1223-32 and Farmaco, Edizione Scientifica (1964) 19(9):741-50). Amide reduction forms 29 and subsequent deprotection yields 33. Alternatively, compound 33 can be formed from carbaldehyde 30. Condensation between 30 and nitromethane yields nitroalkene 31. Reduction to the azatryptamine 32 followed by a Pictet-Spangler cyclization yields 33. A third method for producing 33 is to start from azaindole 34 and perform a Friedel-Crafts alkylation with ethyl oxalyl chloride to form the oxalate ester, which is immediately treated with NH₃ in MeOH to form amide 35 (see Journal of Organic Chemistry (2002) 67(17):6226-27; PCT International Application Publication Number WO 2006/061212 and US Patent Application Publication Number 2004-0192718). Reduction of the amide forms 32 which is cyclized using a Pictet-Spangler cyclization to yield 33. Alkylation with carboxamide 3 or a related diamine under standard alkylation conditions provides 36 and 36′.

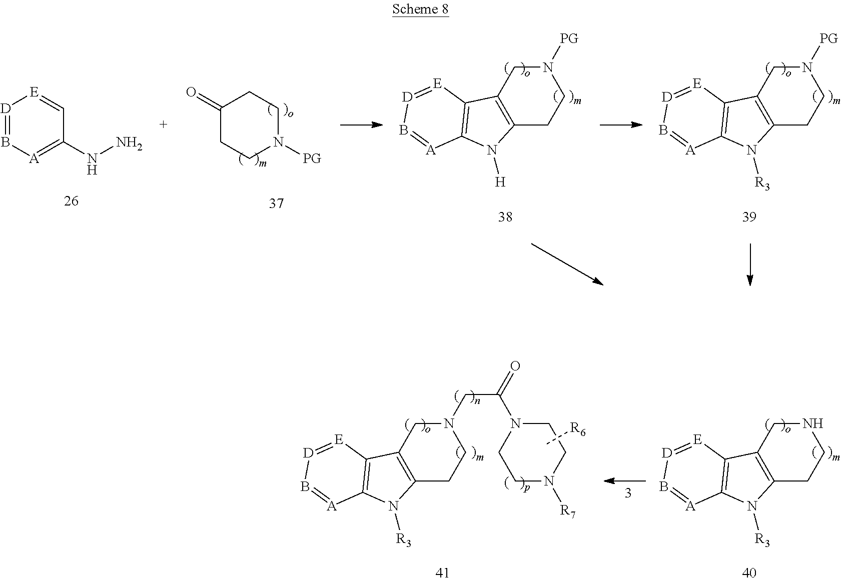

Scheme 8 illustrates the preparation of indole analogues 41. Compound 38 is formed by a Fisher indole reaction between 26 and 37 (Journal of Heterocyclic Chemistry (2006) 43(3):571-78). Deprotection of 38 yields 40. The nitrogen of indole 38 can be alkylated directly with an alkyl-halide or arylated by a Buchwald reaction to form indole 39. Deprotection of 39 forms amine 40. Alternatively, a number of 2,3,4,5-tetrahydro-1H-pyrido[4,3-b]indoles are commercially available (e.g., from AKos Consulting and Solutions GmbH). Alkylation of 40 with carboxamide 3 under standard alkylation conditions provides 41.

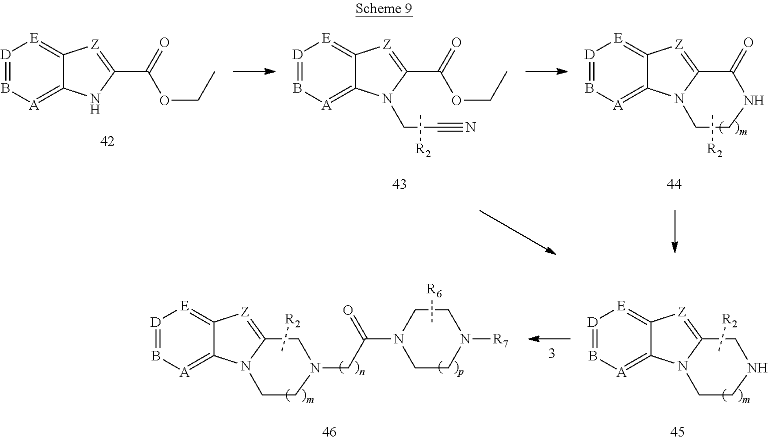

Scheme 9 illustrates the preparation of compounds 46. Starting from the appropriately substituted indole 42, alkylation with chloroacetonitrile forms 43. Either direct reduction with LiAlH₄ to form 45 or stepwise reduction of the nitrile and cyclization to form 44 followed by reduction forms 45 (see Guandalini et al., Archive for Organic Chemistry (ARCHIVOC) 2004(v):286-300 and Bioorganic & Medicinal Chemistry Letters (2004) 14(4):1003-05). Alkylation of 45 with carboxamide 3 under standard alkylation conditions provides 46.

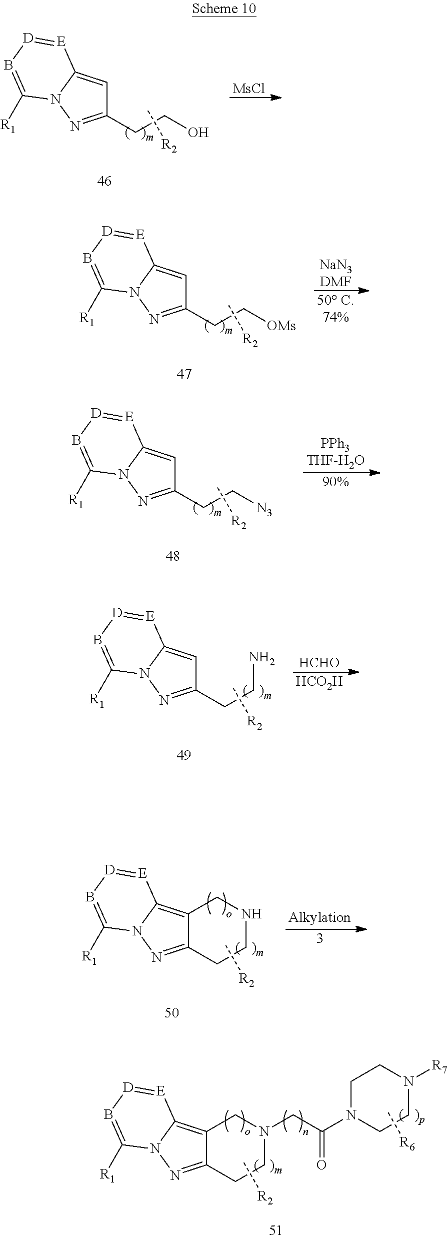

Scheme 10 illustrates the method for preparing 51. Alcohol 46 is known in the literature (e.g., Bioorg. Med. Chem. Leu. (2002) 20:2377-80) or conveniently prepared by a variety of methods familiar to those skilled in the art. Compound 46 is treated with methanesulfonyl chloride to give 47, which is converted to 48 upon treatment with sodium azide in DMF. The azido compound 48 is reduced by triphenylphosphine to give amine 49, which is transformed to 50 through a Pictet-Spengler cyclization. Alkylation of 50 with carboxamide 3 under standard alkylation conditions provides 51.

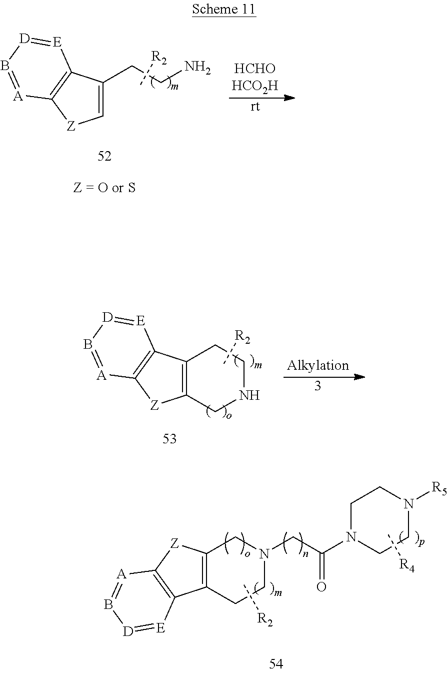

Compounds 54 may be prepared in accordance with Scheme 11. Amine 52 is available from commercial sources such as Aldrich (St Louis, Mo.) or may be synthesized from commercially available precursors using established protocols or readily apparent variations thereon. Treatment of 52 with paraformaldehyde in formic acid under Pictet-Spengler cyclization conditions provides compound 53. Treatment of 53 with carboxamide 3 in the presence of a base such as potassium carbonate produces compound 54.

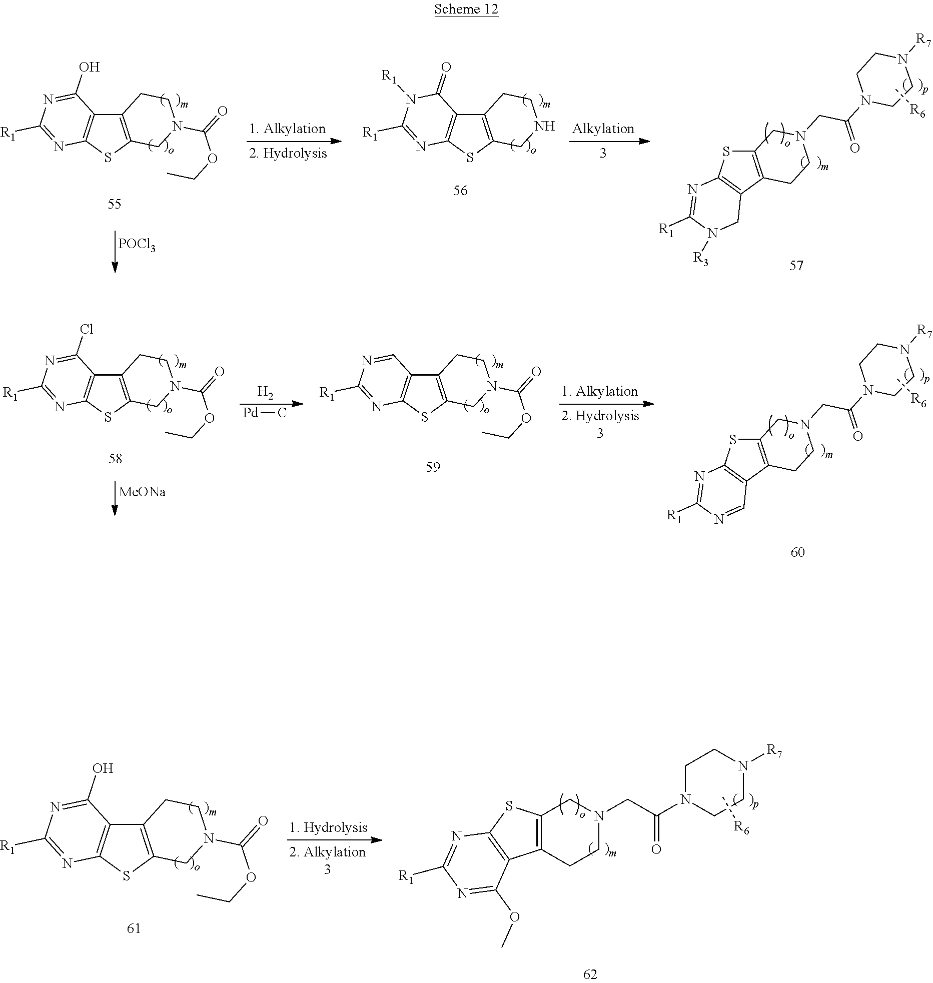

Scheme 12 illustrates the preparation of compounds 57, 60 and 62. The tricyclic intermediate 55 is commercially available, known in the literature (e.g., Ach. Mod. Chem. (1994) 131:489-98) or conveniently prepared by a variety of methods. Treatment of compound 55 with an alkyl halide in acetonitrile followed by deprotection gives pyrimidone 56. Alkylation of 56 with carboxamide 3 under standard alkylation conditions provides 57. Compound 55 can also be converted to chloride compound 58 upon treatment with phosphorous oxychloride in the presence of a base such as pyridine at 100° C.; hydrogenation of compound 58 in the presence of palladium on charcoal provides compound 59, which is transformed to compound 60 upon deprotection followed by a standard alkylation reaction with carboxamide 3. Compound 58 is heated with sodium methoxide in methanol to give intermediate 61, which upon deprotection and reaction with 3 is converted to compound 62.

In certain embodiments, a piperazinyl oxoalkyl tetrahydro-beta-carboline or related analogue provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Piperazinyl oxoalkyl tetrahydro-beta-carbolines and related analogues may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium-labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

### Pharmaceutical Compositions

The present invention also provides pharmaceutical compositions comprising one or more piperazinyl oxoalkyl tetrahydro-beta-carboline or related analogues provided herein, together with at least one physiologically acceptable carrier or excipient. Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs or cats). In addition, other active ingredients may (but need not) be included in the pharmaceutical compositions provided herein.

Pharmaceutical compositions may be formulated for any appropriate manner of administration, including, for example, inhalation (e.g., nasal or oral), topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents to increase the bulk weight of the material to be tableted (e.g., calcium carbonate, sodium carbonate, lactose, calcium phosphate or sodium phosphate), granulating and disintegrating agents that modify the disintegration rate in the environment of use (e.g., corn starch, starch derivatives, alginic acid and salts of carboxymethylcellulose), binding agents that impart cohesive qualities to the powdered material(s) (e.g., starch, gelatin, acacia and sugars such as sucrose, glucose, dextrose and lactose) and lubricating agents (e.g., magnesium stearate, calcium stearate, stearic acid or talc). Tablets may be formed using standard techniques, including dry granulation, direct compression and wet granulation. The tablets may be uncoated or they may be coated by known techniques.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active material(s) in admixture with one or more suitable excipients, such as suspending agents (e.g., sodium carboxymethylcellulose, methylcellulose, hydroxypropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, such as ethyl or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be formulated as oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectable aqueous or oleaginous suspension. The active ingredient(s), depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectable compositions, and adjuvants such as local anesthetics, preservatives and/or buffering agents can be dissolved in the vehicle.

Pharmaceutical compositions may also be prepared in the form of suppositories (e.g., for rectal administration). Such compositions can be prepared by mixing the drug with a suitable non-irritating excipient that is solid at ordinary temperatures but liquid at the body temperature and will therefore melt in the body to release the drug. Suitable excipients include, for example, cocoa butter and polyethylene glycols.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated for release at a pre-determined rate. Instantaneous release may be achieved, for example, via sublingual administration (i.e., administration by mouth in such a way that the active ingredient(s) are rapidly absorbed via the blood vessels under the tongue rather than via the digestive tract). Controlled release formulations (i.e., formulations such as a capsule, tablet or coated tablet that slows and/or delays release of active ingredient(s) following administration) may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours. Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, dibutyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed my methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217.

In addition to, or together with, the above modes of administration, a piperazinyl oxoalkyl tetrahydro-beta-carboline or related analogue provided herein may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Piperazinyl oxoalkyl tetrahydro-beta-carbolines and related analogues provided herein are generally present within a pharmaceutical composition at levels providing a therapeutically effective amount upon administration, as described above. Dosage forms providing dosage levels ranging from about 0.1 mg to about 140 mg per kilogram of body weight per day are preferred (about 0.5 mg to about 7g per human patient per day). The amount of active ingredient that may be combined with the carrier materials to produce a single dosage form will vary depending upon the host treated and the particular mode of administration. Dosage unit forms generally contain between from about 0.1 mg to about 2g, preferably 0.5 mg to 1g, and more preferably 1 mg to 500 mg, of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating conditions responsive to H3 receptor modulation, including those specifically recited herein. Packaged pharmaceutical preparations comprise a container holding one or more dosage units comprising a therapeutically effective amount of at least one H3 receptor modulator as described herein and instructions (e.g., labeling) indicating that the contained composition is to be used for treating a condition responsive to H3 receptor modulation in the patient.

### Methods of Use

H3 receptor modulators provided herein may be used to alter activity and/or activation of H3 receptors in a variety of contexts, both in vitro and in vivo. Within certain aspects, H3 receptor modulators may be used to inhibit or enhance (preferably to inhibit) H3 receptor activity in vitro or in vivo. In general, such methods comprise the step of contacting a H3 receptor with one or more H3 receptor modulators provided herein, in aqueous solution and under conditions otherwise suitable for binding of the modulator(s) to H3 receptor. The H3 receptor modulator(s) are generally present at a concentration that is sufficient to alter H3 receptor GTP binding activity in vitro (using the assay provided in Example 7). The H3 receptor may be present in solution or suspension (e.g., in an isolated membrane or cell preparation), or in a cultured or isolated cell. Within certain embodiments, the H3 receptor is present in a patient (e.g., expressed by a neuronal cell), and the aqueous solution is a body fluid. Preferably, one or more H3 receptor modulators are administered to a patient in an amount such that each H3 receptor modulator is present in at least one body fluid of the patient at a therapeutically effective concentration that is 1 micromolar or less; preferably 500 nanomolar or less; more preferably 100 nanomolar or less, 50 nanomolar or less, 20 nanomolar or less, or 10 nanomolar or less. For example, such compounds may be administered at a dose that is less than 20 mg/kg body weight, preferably less than 5 mg/kg and, in some instances, less than 1 mg/kg. In vivo, modulation of H3 receptor activity may be assessed by detecting an alteration of a symptom (e.g., memory or attention) in a patient being treated with one or more H3 receptor modulators provided herein.

The present invention further provides methods for treating conditions responsive to H3 receptor modulation. Within the context of the present invention, the term “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). A condition is “responsive to H3 receptor modulation” if it is characterized by inappropriate activity of H3 receptor, regardless of the amount of H3 receptor ligand present locally, and/or if modulation of H3 receptor activity results in alleviation of the condition or a symptom thereof. Such conditions may be diagnosed and monitored using criteria that have been established in the art. Patients may include humans, domesticated companion animals and livestock, with dosages as described above.

Conditions that are responsive to H3 receptor modulation include, for example:
Cardiovascular disorders, including atherosclerosis, hypertension, myocardial infarction, coronary heart disease and stroke;
Cancer (e.g., endometrial, breast, prostate and colon cancer, cutaneous carcinoma, medullary thyroid carcinoma and melanoma);
Metabolic disorders including impaired glucose tolerance, dyslipidaemia, and diabetes (e.g., non-insulin dependent diabetes mellitus);
Immune conditions and disorders including osteoarthritis, allergy (e.g., allergic rhinitis), and inflammation;
Respiratory conditions including nasal congestion, upper airway allergic response, asthma and chronic obstructive pulmonary disease;
Disorders associated with the regulation of sleep and wakefulness, or arousal and vigilance, including excessive daytime sleepiness (EDS), shift work disorder, narcolepsy, jet lag, and sleep disorders such as primary insomnia, idiopathic hypersomnia, circadian rhythm sleep disorder, dyssomnia NOS, parasomnias including nightmare disorder, sleep terror disorder, sleep disorders secondary to depression, anxiety and/or other mental disorders and substance-induced sleep disorder;
Fatigue and fatigue-related disorders such as sleep/fatigue disorders, sleep impairment due to perimenopausal hormonal shifts, Parkinson's-related fatigue, multiple sclerosis-related fatigue, and chemotherapy-induced fatigue;
Eating disorders (e.g., bulimia, binge eating and anorexia) and obesity;
Digestive system and gastrointestinal disorders including gallbladder disease, ulcer, hyper- and hypo-motility of the gastrointestinal tract and irritable bowel syndrome;
CNS disorders including hyper- and hypo-activity of the central nervous system, migraine, epilepsy, seizures, convulsions, mood disorders, attention deficit disorder, attention deficit hyperactivity disorder, bipolar disorder, depression, manic disorders, obsessive compulsive disorder, schizophrenia, migraine, vertigo, motion sickness, dementia, cognitive deficit (e.g., in psychiatric disorder, such as mild cognitive impairment), learning deficit, memory deficit (e.g., age-related memory dysfunction), multiple sclerosis, Parkinson's disease, Alzheimer's disease and other neurodegenerative disorders, addiction (e.g., resulting from drug abuse), neurogenic inflammation and Tourette's syndrome;
Vestibular dysfunction (e.g., Meniere's disease, dizziness and motion sickness);
Pain (e.g., inflammatory pain or neuropathic pain) and itch;
Septic shock; and
Glaucoma.

H3 receptor modulators may further be used to enhance a patient's cognitive ability.

In certain embodiments, piperazinyl oxoalkyl tetrahydro-beta-carbolines and related analogues provided herein are used to treat Alzheimer's disease, Parkinson's disease, schizophrenia, mood and attention alterations including attention deficit hyperactivity disorder and attention deficit disorder, memory and learning disorders, cognitive disorders (such as mild cognitive impairment and cognitive deficits in psychiatric pathologies), epilepsy, migraine, and disorders associated with the regulation of sleep and wakefulness, as well as in the treatment and prevention of conditions such as obesity, eating disorders, diabetes, vertigo, motion sickness and allergic rhinitis Treatment regimens may vary depending on the compound used and the particular condition to be treated. However, for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, and rate of excretion, drug combination and the severity of the particular disease undergoing therapy. In general, the use of the minimum dose sufficient to provide effective therapy is preferred. Patients may generally be monitored for therapeutic effectiveness using medical or veterinary criteria suitable for the condition being treated or prevented.

Within other aspects, H3 receptor modulators provided herein may be used within combination therapy for the treatment of conditions that are responsive to H3 receptor modulation, as described above. Alternately, such H3 modulators may be used in combination with drugs that are associated with induction of sleepiness as an adverse effect, so as to limit induction of sleepiness. Within such combination therapy, a H3 receptor modulator is administered to a patient along with a second therapeutic agent that is not a H3 receptor modulator. The H3 receptor modulator and second therapeutic agent may be present in the same pharmaceutical composition, or may be administered separately in either order. It will be apparent that additional therapeutic agents may, but need not, also be administered.

Second therapeutic agents suitable for use in such combination therapy include, for example, antiobesity agents, antidiabetics, antihypertensive agents, antidepressants, antipsychotic agents and anti-inflammatory agents. In certain combinations, the second therapeutic agent is a compound for the treatment of attention deficit disorder or attention deficit hyperactivity disorder, an antipsychotic agent or an anti-obesity agent.

Histamine H1 receptor modulators represent one class of second therapeutic agents. Combination with H1 receptor modulators may be used, for example, in the treatment of Alzheimer's disease, inflammatory diseases and allergic conditions. Representative H1 receptor antagonists include, for example, loratadine, desloratadine, fexofenadine and cetirizine. Other H1 receptor antagonists include ebastine, mizolastine, acrivastine, astemizole, azatadine, azelastine, brompheniramine, chlorpheniramine, clemastine, cyproheptadine, dexchlorpheniramine, diphenhydramine, hydroxyzine, levocabastine, promethazine and tripelenamine. Many of these agents are known to induce sleepiness as an adverse effect.

Antiobesity therapeutic agents for use in combination therapy include, for example, leptin, leptin receptor agonists, melanin concentrating hormone (MCH) receptor antagonists, melanocortin receptor 3 (MC3) agonists, melanocortin receptor 4 (MC4) agonists, melanocyte stimulating hormone (MSH) agonists, cocaine and amphetamine regulated transcript (CART) agonists, dipeptidyl aminopeptidase inhibitors, a growth hormone secretagogue, beta-3 adrenergic agonists, 5HT-2 agonists, orexin antagonists, neuropeptide Y₁ or Y₅ antagonists, tumor necrosis factor (TNF) agonists, galanin antagonists, urocortin agonists, cholecystokinin (CCK) agonists, GLP-1 agonists, serotonin (5HT) agonists, bombesin agonists, CB1 antagonists such as rimonabant, growth hormone, growth factors such as prolactin or placental lactogen, growth hormone releasing compounds, thyrotropin (TRH) agonists, uncoupling protein 2 or 3 (UCP 2 or 3) modulators, dopamine agonists (e.g., the partial D2 agonist aplindore), agents that modify lipid metabolism such as antilipidemic agents (e.g., cholestyramine, colestipol, clofibrate, gemfibrozil, lovastatin, pravastatin, simvastatin, probucol or dextrothyroxine), lipase/amylase inhibitors, peroxisome proliferator-activated receptor (PPAR) modulators, retinoid X receptor (RXR) modulators, TR-beta agonists, agouti-related protein (AGRP) inhibitors, opioid antagonists such as naltrexone, exendin-4, GLP-1, ciliary neurotrophic factor, corticotropin-releasing factor binding protein (CRF BP) antagonists and/or corticotropin-releasing factor (CRF) agonists. Representative such agents include, for example, sibutramine, dexfenfluramine, dextroamphetamine, amphetamine, orlistat, mazindol, phentermine, phendimetrazine, diethylpropion, fluoxetine, bupropion, topiramate and ecopipam.

Antihypertensive therapeutic agents for use in combination therapy include, for example, beta-blockers such as alprenolol, atenolol, timolol, pindolol, propranolol and metoprolol, angiotensin converting enzyme (ACE) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, alpha-blockers such as doxazosin, urapidil, prazosin and terazosin, and angiotensin receptor blockers such as losartan.

CNS-active agents for use in combination therapy include, but are not limited to the following, many of which are know to induce sleepiness as an adverse effect: for anxiety, depression, mood disorders or schizophrenia—serotonin receptor (e.g., 5-HT_{1A}) agonists and antagonists, neurokinin receptor antagonists, GABAergic agents, and corticotropin releasing factor receptor (CRF₁) antagonists; for sleep disorders—melatonin receptor agonists; and for neurodegenerative disorders—such as Alzheimer's dementia, nicotinic agonists, muscarinic agents, acetylcholinesterase inhibitors and dopamine receptor agonists. For example, such combination therapy may include a selective serotonin reuptake inhibitor (SSRI) or a non-selective serotonin, dopamine and/or norepinephrine reuptake inhibitor. Such agents include, for example, fluoxetine, sertraline, paroxetine, amitriptyline, seroxat and citalopram. For cognitive disorders, representative agents for use in combination therapy include GABAergic agents.

Other therapeutic agents suitable for combination therapy include, for example, agents that modify cholinergic transmission (e.g., 5-HT₆ antagonists), M1 muscarinic agonists, M2 muscarinic antagonists and acetylcholinesterase inhibitors.

Suitable doses for H3 receptor modulator within such combination therapy are generally as described above. Doses and methods of administration of other therapeutic agents can be found, for example, in the manufacturer's instructions in the Physician's Desk Reference. In certain embodiments, the combination administration of a H3 receptor modulator with the second therapeutic agent results in a reduction of the dosage of the second therapeutic agent required to produce a therapeutic effect (i.e., a decrease in the minimum therapeutically effective amount). Thus, preferably, the dosage of second therapeutic agent in a combination or combination treatment method is less than the maximum dose advised by the manufacturer for administration of the second therapeutic agent without combination administration of a H3 receptor modulator. More preferably this dosage is less than ¾, even more preferably less than ½, and highly preferably, less than ¼ of the maximum dose, while most preferably the dose is less than 10% of the maximum dose advised by the manufacturer for the second therapeutic agent when administered without combination administration of a H3 receptor modulator. It will be apparent that the dosage amount of H3 receptor modulator component(s) of the combination needed to achieve the desired effect may similarly be affected by the dosage amount and potency of the other therapeutic component(s) of the combination.

In certain preferred embodiments, the combination administration of a H3 receptor modulator with other therapeutic agent(s) is accomplished by packaging one or more H3 receptor modulators and one or more other therapeutic agents in the same package, either in separate containers within the package or in the same contained as a mixture of one or more H3 receptor modulators and one or more other therapeutic agents. Preferred mixtures are formulated for oral administration (e.g., as pills, capsules, tablets or the like). In certain embodiments, the package comprises a label bearing indicia indicating that the one or more H3 receptor modulators and one or more other therapeutic agents are to be taken together for the treatment of attention deficit disorder, attention deficit hyperactivity disorder, schizophrenia, a cognitive disorder (such as mild cognitive impairment), epilepsy, migraine, a sleep disorder, excessive daytime sleepiness (EDS), shift work disorder, narcolepsy, allergic rhinitis, vertigo, motion sickness, a memory disorder such as Alzheimer's disease, Parkinson's disease, obesity, an eating disorder or diabetes.

Within separate aspects, the present invention provides a variety of non-pharmaceutical in vitro and in vivo uses for the piperazinyl oxoalkyl tetrahydro-beta-carbolines and related analogues provided herein. For example, such compounds may be labeled and used as probes for the detection and localization of H3 receptor (in samples such as cell preparations or tissue sections, preparations or fractions thereof). In addition, compounds provided herein that comprise a suitable reactive group (such as an aryl carbonyl, nitro or azide group) may be used in photoaffinity labeling studies of receptor binding sites. Compounds provided herein may further be used as positive controls in assays for receptor activity, as standards for determining the ability of a candidate agent to bind to H3 receptor, or as radiotracers for positron emission tomography (PET) imaging or for single photon emission computerized tomography (SPECT). Such methods can be used to characterize H3 receptors in living subjects. For example, a piperazinyl oxoalkyl tetrahydro-beta-carboline or related analogue may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound piperazinyl oxoalkyl tetrahydro-beta-carboline or related analogue is removed (e.g., by washing), and bound piperazinyl oxoalkyl tetrahydro-beta-carboline or related analogue detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of H3 receptor in the sample. Detection assays, including receptor autoradiography (receptor mapping) of H3 receptor in cultured cells or tissue samples may be performed as described by Kuhar in sections 8.1.1 to 8.1.9 of Current Protocols in Pharmacology (1998) John Wiley & Sons, New York.

Piperazinyl oxoalkyl tetrahydro-beta-carbolines and related analogues provided herein may also be used within a variety of well known cell separation methods. For example, modulators may be linked to the interior surface of a tissue culture plate or other support, for use as affinity ligands for immobilizing and thereby isolating, H3 receptors (e.g., isolating receptor-expressing cells) in vitro. Within one preferred embodiment, a modulator linked to a fluorescent marker, such as fluorescein, is contacted with the cells, which are then analyzed (or isolated) by fluorescence activated cell sorting (FACS).

Piperazinyl oxoalkyl tetrahydro-beta-carbolines and related analogues provided herein may further be used within assays for the identification of other agents that bind to H3 receptor. In general, such assays are standard competition binding assays, in which bound, labeled piperazinyl oxoalkyl tetrahydro-beta-carboline or related analogue is displaced by a test compound. Briefly, such assays are performed by: (a) contacting H3 receptor with a radiolabeled piperazinyl oxoalkyl tetrahydro-beta-carboline or related analogue as described herein, under conditions that permit binding of the piperazinyl oxoalkyl tetrahydro-beta-carboline or related analogue to H3 receptor, thereby generating bound, labeled piperazinyl oxoalkyl tetrahydro-beta-carboline or related analogue; (b) detecting a signal that corresponds to the amount of bound, labeled piperazinyl oxoalkyl tetrahydro-beta-carboline or related analogue in the absence of test agent; (c) contacting the bound, labeled piperazinyl oxoalkyl tetrahydro-beta-carboline or related analogue with a test agent; (d) detecting a signal that corresponds to the amount of bound, labeled piperazinyl oxoalkyl tetrahydro-beta-carboline or related analogue in the presence of test agent; and (e) detecting a decrease in signal detected in step (d), as compared to the signal detected in step (b), and therefrom identifying an agent that binds to H3 receptor.

The following Examples are offered by way of illustration and not by way of limitation. Unless otherwise specified all reagents and solvent are of standard commercial grade and are used without further purification. Using routine modifications, the starting materials may be varied and additional steps employed to produce other compounds provided herein.

### EXAMPLES

Mass spectroscopy data in this and the following Examples is Electrospray MS, obtained in positive ion mode using a Micromass Time-of-Flight LCT (Micromass, Beverly Mass.), equipped with a Waters 600 pump (Waters Corp., Milford, Mass.), Waters 996 photodiode array detector, Gilson 215 autosampler (Gilson, Inc. Middleton, Wis.), and a Gilson 841 microinjector. MassLynx (Advanced Chemistry Development, Inc; Toronto, Canada) version 4.0 software with OpenLynx processing is used for data collection and analysis. MS conditions are as follows: capillary voltage=3.5 kV; cone voltage=30 V, desolvation and source temperature=350° C. and 120° C., respectively; mass range=181-750 with a scan time of 0.22 seconds and an interscan delay of 0.05 min.

Sample volume of 1 microliter is injected onto a 50×4.6 mm Chromolith SpeedROD RP-18e column (Merck KGaA, Darmstadt, Germany), and eluted using a 2-phase linear gradient at 6 mL/min flow rate. Sample is detected using total absorbance count over the 220-340 nm UV range. The elution conditions are: Mobile Phase A-95/5/0.05 Water/MeOH/TFA; Mobile Phase B-5/95/0.025 Water/MeOH/TFA. The following gradient is used, with an inject to inject cycle of 2.2 min: 0-0.5 minutes 10-100% B, hold at 100% B to 1.2 minutes, return to 10% B at 1.21 minutes.

### Example 1

### Preparation of Representative Compounds

### 1. 7-[2-(4-CYCLOBUTYLPIPERAZIN-1-YL)-2-OXOETHYL]-5,6,7,8-TETRAHYDRO PYRAZOLO[1,5-A]PYRIDO[4,3-D]PYRIMIDINE (SCHEME 2)

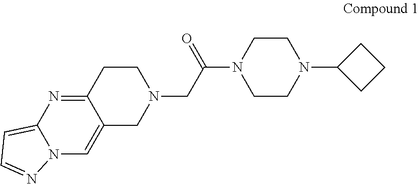

### Step 1. tert-Butyl 7,8-dihydropyrazolo[1,5-a]pyrido[3,4-d]pyrimidine-6(5H)-carboxylate

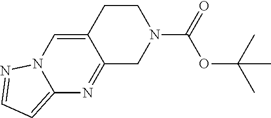

To a solution of 3-dimethylaminomethylene-4-oxo-piperidine-1-carboxylic acid tert-butyl ester (775 mg, 3.05 mmol) in anhydrous DMF (10 mL) is added 5-aminopyrazole (253 mg, 3.05 mmol). The mixture is heated at 120° C. overnight. The mixture is cooled to RT and partitioned between Et₂O (100 mL) and H₂O (100 mL), followed by extraction with Et₂O (2×100 mL). The organic extracts are combined, washed with water (100 mL) and brine (100 mL), dried and evaporated to give the crude product. MS (+VE) m/z 275.21 (M⁺+1).

### Step 2. 5,6,7,8-Tetrahydropyrazolo[1,5-a]pyrido[3,4-d]pyrimidine

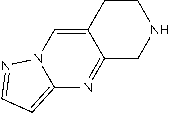

tert-Butyl 7,8-dihydropyrazolo[1,5-a]pyrido[3,4-d]pyrimidine-6(5H)-carboxylate (327 mg, 1.20 mmol) is dissolved in 4 N HCl in dioxane (5 mL) and stirred at rt for 4 h. The solvent is removed under reduced pressure to yield the title compound as its hydrochloride salt. MS (+VE) m/z 175.11 (M⁺+1).

### Step 3. 7-[2-(4-Cyclobutylpiperazin-1-yl)-2-oxoethyl]-5,6,7,8-tetrahydropyrazolo[1,5-a]pyrido[4,3-d]pyrimidine

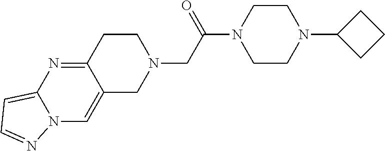

To a solution of 5,6,7,8-tetrahydropyrazolo[1,5-a]pyrido[3,4-d]pyrimidine hydrochloride (250 mg, 1.19 mmol) in acetonitrile (5 mL) is added NaI (50 mg, 0.333 mmol), K₂CO₃ (400 mg, 2.90 mmol) and 1-(chloroacetyl)-4-cyclobutylpiperazine (257 mg, 1.19 mmol). The mixture is stirred overnight at rt. The mixture is diluted with DCM (25 mL) and filtered through Celite. The filtrate is concentrated and the residue is purified by PTLC eluting with EtOAc/MeOH/TEA (95:5:5) to yield the title compound. ¹H NMR (300 MHz, CDCl₃) δ 8.24 (1H, s), 8.11 (1H, d), 6.71 (1H, d), 3.81 (2H, s), 3.65-3.58 (4H, m), 3.50 (2H, s), 3.31 (2H, t), 3.04 (2H, t), 2.65 (1H, m), 2.36-2.30 (4H, m), 2.09-1.67 (m, 6H); MS (+VE) m/z 355.17 (M⁺+1).

### 2. 6-[2-(4-CYCLOBUTYLPIPERAZIN-1-YL)-2-OXOETHYL]-5,6,7,8-TETRAHYDRO PYRAZOLO[1,5-A]PYRIDO[3,4-D]PYRIMIDINE (Scheme 3)

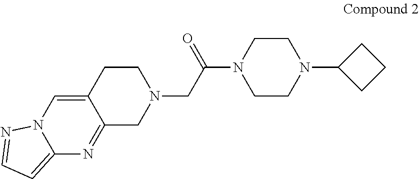

### Step 1. tert-Butyl 7,8-dihydropyrazolo[1,5-a]pyrido[3,4-d]pyrimidine-6(5H)-carboxylate

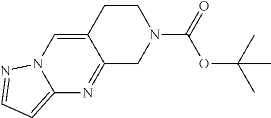

To a solution of tert-butyl-4-[(dimethylamino)methylene]-3-oxopiperidine-1-carboxylate (1.20 g, 4.22 mmol) in anhydrous DMF (10 mL) is added 5-aminopyrazole (509 g, 6.13 mmol) and K₂CO₃ (2.33 g, 16.9 mmol). The mixture is heated at 120° C. overnight. The mixture is cooled to rt, partitioned between Et₂O (100 mL) and H₂O (100 mL) and extracted with Et₂O (2×100 mL). The organic extracts are combined, and washed with water (100 mL) and brine (100 mL). The organic extract is dried and evaporated to give the title compound. MS (+VE) m/z 275.20 (M⁺+1).

### Step 2. 5,6,7,8-Tetrahydropyrazolo[1,5-a]pyrido[3,4-d]pyrimidine

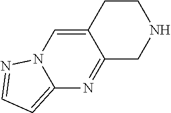

tert-Butyl 7,8-dihydropyrazolo[1,5-a]pyrido[3,4-d]pyrimidine-6(5H)-carboxylate (140 mg, 0.51 mmol) is dissolved in TFA (5 mL) and stirred at rt for 3 h. The solvent is removed under reduced pressure to yield the title compound as its trifluoroacetic acid salt. MS (+VE) m/z 175.12 (M⁺+1).

### Step 3. 6-[2-(4-Cyclobutylpiperazin-1-yl)-2-oxoethyl]-5,6,7,8-tetrahydropyrazolo[1,5-a]pyrido[3,4-d]pyrimidine

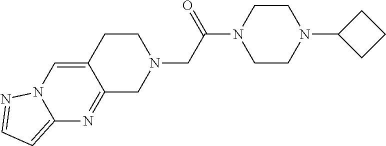

To a solution of 5,6,7,8-tetrahydropyrazolo[1,5-a]pyrido[3,4-d]pyrimidine trifluoroacetate (146 mg, 0.51 mmol) in acetonitrile (5 mL) is added NaI (50 mg, 0.333 mmol), K₂CO₃ (750 mg, 5.42 mmol) and 1-(chloroacetyl)-4-cyclobutylpiperazine (120 mg, 0.56 mmol). The mixture is stirred overnight at rt. The mixture is diluted with DCM (25 mL) and filtered through Celite. The filtrate is concentrated and the residue is purified by PTLC eluting with EtOAc/MeOH/TEA (90:10:5) to yield the title compound. ¹H NMR (300 MHz, CDCl₃) δ 8.09 (1H, s), 7.96 (1H, d), 6.59 (1H, d), 4.17 (2H, s), 3.64-3.57 (4H, m), 3.52 (2H, t), 3.49 (2H, s), 3.31 (2H, t), 2.65 (1H, m), 2.36-2.30 (4H, m), 2.07-1.65 (m, 6H); MS (+VE) m/z 355.15 (M⁺+1).

### 3. 2-[2-(4-CYCLOBUTYLPIPERAZIN-1-YL)-2-OXOETHYL]-2,3,4,9-TETRAHYDRO-1H-BETA-CARBOLINE (SCHEME 4)

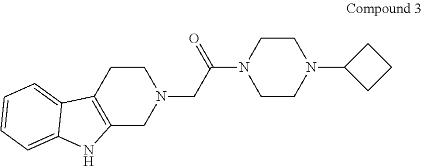

To a solution of 2,3,4,9-tetrahydro-1H-beta-carboline (Aldrich; 150 mg, 0.87 mmol) in acetonitrile (10 mL) is added NaI (50 mg, 0.333 mmol), K₂CO₃ (300 mg, 2.17 mmol) and 1-(chloroacetyl)-4-cyclobutylpiperazine (226 mg, 1.05 mmol). The mixture is stirred overnight at rt. The mixture is diluted with DCM (25 mL) and filtered through Celite. The filtrate is concentrated and the residue is purified by PTLC eluting with EtOAc/MeOH/TEA (90:10:5) to yield the title compound. ¹H NMR (300 MHz, CDCl₃) δ 8.29 (1H, br ex s), 7.45 (1H, d), 7.30 (1H, d), 7.09 (2H, m), 3.75 (2H, s), 3.66-3.61 (4H, m), 3.43 (2H, s), 2.91 (2H, t), 2.80 (2H, t), 2.69 (1H, m), 2.29-2.28 (4H, m), 2.04-1.99 (2H, m), 1.89-1.83 (2H, m); 1.73-1.67 (2H, m); MS (+VE) m/z 353.14 (M⁺+1).

### 4. 2-[2-(4-CYCLOBUTYLPIPERAZIN-1-YL)-2-OXOETHYL]-9-METHYL-2,3,4,9-TETRAHYDRO-1H-BETA-CARBOLINE (SCHEME 4)

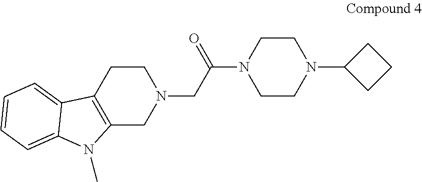

### Step 1. tert-Butyl 1,3,4,9-tetrahydro-2H-beta-carboline-2-carboxylate

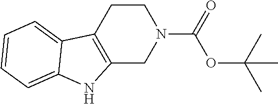

To a solution of 2,3,4,9-tetrahydro-1H-beta-carboline (300 mg, 1.74 mmol) in THF (10 mL) and 1N NaOH (2 mL) is added di-t-butyl dicarbonate (380 mg, 1.80 mmol). The mixture is stirred 3 h at rt. EtOAc (50 mL) is added and the mixture is extracted with 1N NaOH (2×50 mL). The organic extract is dried over Na₂SO₄ and evaporated under reduced pressure to yield the title compound. MS (+VE) m/z 273.25 (M⁺+1).

### Step 2. tert-Butyl 9-methyl-1,3,4,9-tetrahydro-2H-beta-carboline-2-carboxylate

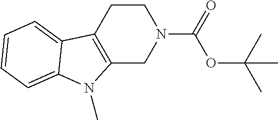

To a solution of tert-butyl 1,3,4,9-tetrahydro-2H-beta-carboline-2-carboxylate (337 mg, 1.24 mmol) in DMF (10 mL) is added NaH (60%; 94 mg, 2.48 mmol). The mixture is stirred one half hour at rt. Iodomethane (0.115 mL, 1.86 mmol) is added and the mixture is allowed to stir 1 h. Water (50 mL) is added and the mixture is extracted with ether (2×50 mL). The combined organic extracts are washed with water (100 mL) and brine (100 mL), dried over Na₂SO₄ and evaporated under reduced pressure. The residue is purified by silica gel chromatography eluting with hexane/EtOAc (6:1) to yield the title compound. MS (+VE) m/z 287.27 (M⁺+1).

### Step 3. 9-Methyl-2,3,4,9-tetrahydro-1H-beta-carboline

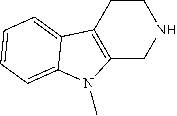

To a solution of tert-butyl 9-methyl-1,3,4,9-tetrahydro-2H-beta-carboline-2-carboxylate (354 mg, 1.24 mmol) in DCM (10 mL) is added trifluoroacetic acid (5 mL). The mixture is stirred 2 h at rt. The solvent is removed under reduced pressure to yield the title compound as its trifluoroacetic acid salt. MS (+VE) m/z 287.27 (M⁺+1).

### Step 4. 2-[2-(4-Cyclobutylpiperazin-1-yl)-2-oxoethyl]-9-methyl-2,3,4,9-tetrahydro-1H-beta-carboline

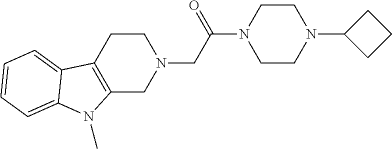

To a solution of 9-methyl-2,3,4,9-tetrahydro-1H-beta-carboline trifluoroacetate (105 mg, 0.35 mmol) in acetonitrile (10 mL) is added NaI (50 mg, 0.333 mmol), K₂CO₃ (400 mg, 2.89 mmol) and 1-(chloroacetyl)-4-cyclobutylpiperazine (105 mg, 0.48 mmol). The mixture is stirred overnight at rt. The mixture is diluted with DCM (25 mL) and filtered through Celite. The filtrate is concentrated and the residue is purified by PTLC eluting with EtOAc/MeOH/TEA (90:10:5) to yield the title compound. ¹H NMR (300 MHz, CDCl₃) δ 7.47 (1H, d), 7.26 (1H, d), 7.17 (1H, t), 7.08 (1H, t), 3.77 (2H, s), 3.67-3.65 (4H, m), 3.59 (3H, s), 3.48 (2H, s), 2.91 (2H, t), 2.82 (2H, t), 2.68 (1H, m), 2.30-2.27 (4H, m), 2.04-1.98 (2H, m), 1.87-1.83 (2H, m); 1.72-1.67 (2H, m); MS (+VE) m/z 367.10 (M⁺+1).

### 5. 2-[2-(4-CYCLOBUTYLPIPERAZIN-1-YL)-2-OXOETHYL]-9-PHENYL-2,3,4,9-TETRAHYDRO-1H-BETA-CARBOLINE (SCHEME 4)

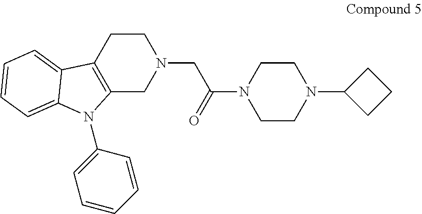

### Step 1. tert-Butyl 9-phenyl-1,3,4,9-tetrahydro-2H-beta-carboline-2-carboxylate

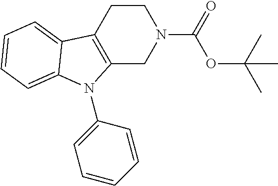

To a solution of tert-butyl 1,3,4,9-tetrahydro-2H-beta-carboline-2-carboxylate (250 mg, 0.92 mmol) in dioxane (5 mL) is added bromobenzene (288 mg, 1.84 mmol), Pd₂ dba₃ (84 mg, 0.092 mmol), Xantphos (53 mg, 0.092 mmol) and Cs₂CO₃ (400 mg, 1.23 mmol). The mixture is degassed with nitrogen and heated at 110° C. overnight. The mixture is cooled to rt and diluted with DCM (25 mL). The mixture is filtered through Celite and the filtrate evaporated under reduced pressure. The residue is purified by silica gel chromatography eluting with hexane/EtOAc (2:1) to yield the title compound. MS (+VE) m/z 349.20 (M⁺+1).

### Step 2. 9-Phenyl-2,3,4,9-tetrahydro-1H-beta-carboline

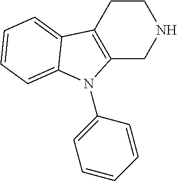

t-Butyl 9-phenyl-1,3,4,9-tetrahydro-2H-beta-carboline-2-carboxylate (268 mg, 0.77 mmol) is dissolved in 4N HCl in dioxane (5 mL) and stirred for 4 h at rt. The solvent is evaporated under reduced pressure to yield the title compound as its hydrochloride salt. MS (+VE) m/z 249.14 (M⁺+1).

### Step 3. 2-[2-(4-Cyclobutylpiperazin-1-yl)-2-oxoethyl]-9-phenyl-2,3,4,9-tetrahydro-1H-beta-carboline

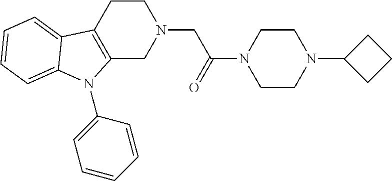

To a solution of 9-phenyl-2,3,4,9-tetrahydro-1H-beta-carboline hydrochloride (270 mg, 0.77 mmol) in acetonitrile (10 mL) is added NaI (50 mg, 0.333 mmol), K₂CO₃ (400 mg, 2.89 mmol) and 1-(chloroacetyl)-4-cyclobutylpiperazine (200 mg, 0.927 mmol). The mixture is stirred overnight at rt. The mixture is diluted with DCM (25 mL) and filtered through Celite. The filtrate is concentrated and the residue is purified by PTLC eluting with EtOAc/MeOH/TEA (95:5:5) to yield the title compound. ¹H NMR (300 MHz, CDCl₃) δ 7.55-7.49 (3H, m), 7.43-7.34 (3H, d), 7.24 (1H, t), 7.16-7.13 (2H, m), 3.65-3.63 (6H, m), 3.42 (2H, s), 2.99 (2H, t), 2.90 (2H, t), 2.72 (1H, m), 2.33-2.30 (4H, m), 2.03-1.98 (2H, m), 1.93-1.86 (2H, m); 1.74-1.68 (2H, m); MS (+VE) m/z 429.13 (M⁺+1).

### 6. 2-[2-(4-CYCLOBUTYLPIPERAZIN-1-YL)-2-OXOETHYL]-6-PYRIMIDIN-5-YL-2,3,4,9-TETRAHYDRO-1H-BETA-CARBOLINE (SCHEME 5)

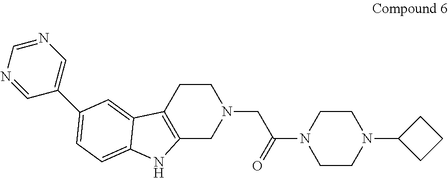

To a solution of 6-bromo-2-[2-(4-cyclobutylpiperazin-1-yl)-2-oxoethyl]-2,3,4,9-tetrahydro-1H-beta-carboline (200 mg, 0.463 mmol) in EtOH (7 mL), toluene (7 mL) and water (2 mL) is added pyrimidine-5-boronic acid (100 mg, 0.811 mmol), K₂CO₃ (256 mg, 1.85 mmol) and Pd(PPh₃)₄ (27 mg, 0.23 mmol). The mixture is degassed with nitrogen and heated in a sealed tube overnight at 95° C. The mixture is cooled to rt and partitioned between EtOAc (50 mL) and 1N NaOH (50 mL). The mixture is extracted with EtOAc (2×50 mL). The combined organic extracts are placed directly on an SCX ion exchange resin and washed first with EtOAc/MeOH (95:5) to waste and second with EtOAc/MeOH/TEA (90:10:10) collected and evaporated. The residue is purified by PTLC eluting with EtOAc/MeOH/TEA (90:10:10) to yield the title compound. ¹H NMR (300 MHz, CDCl₃) δ 9.16 (1H, s), 9.00 (2H, s), 8.23 (1H, br ex s), 7.67 (1H, d), 7.44 (1H, d), 7.34 (1H, dd), 3.82 (2H, s), 3.66 (4H, m), 2.48 (2H, s), 2.97 (2H, t), 2.87 (2H, t), 2.69 (1H, m), 2.30 (4H, m), 2.04-1.97 (2H, m), 1.88-1.82 (2H, m); 1.74-1.68 (2H, m); MS (+VE) m/z 431.14 (M⁺+1).

### 7. 2-[2-(4-CYCLOBUTYLPIPERAZIN-1-YL)-2-OXOETHYL]-2,3,4,9-TETRAHYDRO-1H-BETA-CARBOLINE-6-CARBONITRILE (SCHEME 5)

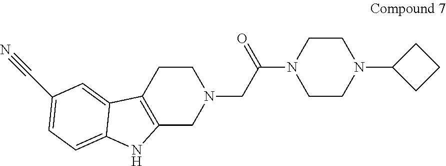

To a solution of 6-bromo-2-[2-(4-cyclobutylpiperazin-1-yl)-2-oxoethyl]-2,3,4,9-tetrahydro-1H-beta-carboline (470 mg, 1.10 mmol) in DMF (6 mL) is added Zn(CN)₂ (102 mg, 0.872 mmol), Pd₂ dba₃ (50 mg, 0.055 mmol) and dppf (60 mg, 0.11 mmol). The mixture is degassed with nitrogen and heated in a sealed tube overnight at 120° C. The mixture is cooled to rt and partitioned between EtOAc (50 mL) and 1N NaOH (50 mL). The mixture is extracted with EtOAc (2×50 mL). The combined organic extracts are dried over Na₂SO₄ and evaporated under reduced pressure. The residue is purified by PTLC eluting with EtOAc/MeOH/TEA (95:5:5) to yield the title compound. ¹H NMR (300 MHz, CDCl₃) δ 8.50 (1H, br ex s), 7.63 (1H, d), 7.48 (1H, d), 7.31 (1H, dd), 3.82 (2H, s), 3.66 (4H, m), 2.48 (2H, s), 2.97 (2H, t), 2.87 (2H, t), 2.69 (1H, m), 2.30 (4H, m), 2.04-1.97 (2H, m), 1.88-1.82 (2H, m); 1.74-1.65 (2H, m); MS (+VE) m/z 378.11 (M⁺+1).

### 8. 2-[2-(4-ISOPROPYLPIPERAZIN-1-YL)-2-OXOETHYL]-2,3,4,9-TETRAHYDRO-1H-BETA-CARBOLINE (SCHEME 6)

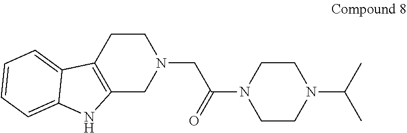

### Step 1. tert-Butyl 1,3,4,9-tetrahydro-2H-beta-carbolin-2-ylacetate

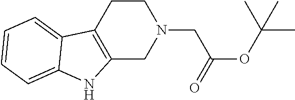

To a solution of 2,3,4,9-tetrahydro-1H-beta-carboline (1.72 mg, 10 mmol) in acetonitrile (50 mL) is added t-butyl bromoacetate (1.95 g, 10 mmol), NaI (500 mg, 3.33 mmol) and K₂CO₃ (2.07 mg, 15 mmol). The mixture is stirred overnight at rt. The mixture is diluted with DCM (50 mL) and filtered through Celite. The filtrate is concentrated and the residue purified by silica gel chromatography eluting with EtOAc/hexane (1:1) to yield the title compound. MS (+VE) m/z 287.12 (M⁺+1).

### Step 2. 1,3,4,9-Tetrahydro-2H-beta-carbolin-2-ylacetic acid

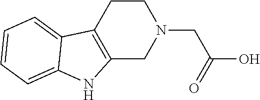

tert-Butyl 1,3,4,9-tetrahydro-2H-beta-carbolin-2-ylacetate (2.7 g, 9.4 mmol) is dissolved in 4N HCl dioxane and heated at 80° C. for 4 h. The mixture is cooled to rt and the solvent removed under reduced pressure to yield the title compound as its hydrochloride salt. MS (+VE) m/z 231.13 (M⁺+1).

### Step 3. 2-[2-(4-isopropylpiperazin-1-yl)-2-oxoethyl]-2,3,4,9-tetrahydro-1H-beta-carboline

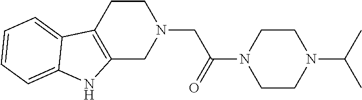

To a solution of 1,3,4,9-tetrahydro-2H-beta-carbolin-2-ylacetic acid (26 mg, 0.96 mmol) in 5% TEA in DMA (0.48 mL), is added 1-isopropylpiperazine (10.3 mg, 0.08 mmol) in 5% TEA in DMA (0.4 mL) and DMC (13.5 mg, 0.08 mmol) in acetonitrile (0.4 mL). The mixture is heated at 50° C. for 5 h. The mixture is cooled to rt and partitioned between EtOAc (1 mL) and 1N NaOH (1 mL). The organic layer is extracted and concentrated under vacuum (˜1 mL) to remove excess TEA. The residue is redissolved in EtOAc, placed directly on an SCX ion exchange resin and washed first with EtOAc/MeOH (95:5) (discarded as waste) and second with EtOAc/MeOH/TEA (90:10:10) which is collected and evaporated to yield the title compound. ¹H NMR (300 MHz, CDCl₃) δ 8.07 (1H, br ex s), 7.47 (1H, d), 7.30 (1H, d), 7.10 (2H, m), 3.78 (2H, s), 3.65 (4H, m), 3.45 (2H, s), 2.94 (2H, t), 2.82 (2H, t), 2.69 (1H, m), 2.49 (4H, m), 1.02 (6H, d); MS (+VE) m/z 341.11 (M⁺+1).

### 9. 7-[2-(4-CYCLOBUTYLPIPERAZIN-1-YL)-2-OXOETHYL]-9-METHYL-6,7,8,9-TETRAHYDRO-5H-PYRIDO[4′,3′:4,5]PYRROLO[2,3-B]PYRIDINE (SCHEME 7)

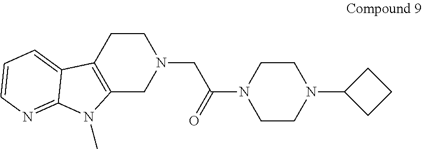

### Step 1. 1-Methyl-1H-pyrrolo[2,3-b]pyridine-3-carbaldehyde

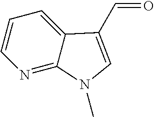

POCl₃ (1.48 mL, 9.65 mmol) is added dropwise to a 0° C. solution of DMF (5 mL). The mixture is stirred 0.5 h at 0° C. In a separate flask, 1-methyl-1H-pyrrolo[2,3-b]pyridine (Journal of the American Chemical Society (2005) 127(22):8050-57; 1.04 g, 7.87 mmol) is dissolved in DMF (10 mL) and added dropwise to the first solution at 0° C. The mixture is allowed to warm to rt and stirred overnight. The mixture is diluted with water (100 mL) and basified with 10 N NaOH. The mixture is extracted with DCM (3×50 mL). The combined organic extracts are washed with water (3×100 mL) and brine (100 mL), dried over Na₂SO₄, and evaporated to yield the title compound. MS (+VE) m/z 161.12 (M⁺+1).

### Step 2. 1-Methyl-3-[(E)-2-nitrovinyl]-1H-pyrrolo[2,3-b]pyridine

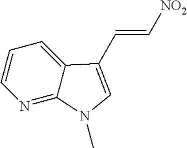

To a solution of 1-methyl-1H-pyrrolo[2,3-b]pyridine-3-carbaldehyde (1.18 g, 7.34 mmol) in nitromethane (20 mL) is added NH₄OAc (200 mg, 2.59 mmol). The mixture is stirred overnight at 70° C. The solution is cooled to rt and concentrated under reduced pressure. The residue is dissolved in EtOAc (50 mL) and extracted with saturated aqueous NaHCO₃ (2×50 mL). The organic extract is dried over Na₂SO₄, and evaporated to yield the title compound. MS (+VE) m/z 204.18 (M⁺+1).

### Step 3. 2-(1-Methyl-1H-pyrrolo[2,3-b]pyridin-3-yl)ethanamine

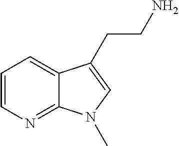

To a 0° C. solution of 1-methyl-3-[(E)-2-nitrovinyl]-1H-pyrrolo[2,3-b]pyridine (1.31 g, 7.00 mmol) in THF (50 mL) is added LiAlH₄ (1.06 mg, 28 mmol). The mixture is allowed to warm to rt and stirred overnight. The solution is cooled to 0° C. and water (1.06 mL), 20% KOH (1.06 mL) and water (3.18 mL) are added sequentially. The mixture is stirred for 0.5 h. Na₂SO₄ is added and the mixture is filtered through Celite. The filter cake is pressed and washed with DCM (2×100 mL). The filtrate is evaporated and the crude product chromatographed on silica eluting with EtOAc/MeOH/TEA (95:5:5) to yield the title compound. MS (+VE) m/z 176.21 (M⁺+1).

### Step 4. 9-Methyl-6,7,8,9-tetrahydro-5H-pyrido[4′,3′:4,5]pyrrolo[2,3-b]pyridine

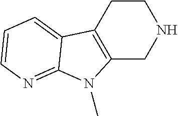

To a solution of 2-(1-methyl-1H-pyrrolo[2,3-b]pyridin-3-yl)ethanamine (210 mg, 1.20 mmol) in formic acid (5 mL) is added paraformaldehyde (36 mg, 1.20 mmol). The mixture is heated at 65° C. for 0.5 h. The mixture is cooled and the solvent removed under reduced pressure. The residue is partitioned between DCM (25 mL) and 1 N NaOH (25 mL). The mixture is extracted with DCM (2×25 mL). The combined organic extracts are dried over Na₂SO₄ and evaporated under reduced pressure to yield the title compound. MS (+VE) m/z 188.22 (M⁺+1).

### Step 5. 7-[2-(4-Cyclobutylpiperazin-1-yl)-2-oxoethyl]-9-methyl-6,7,8,9-tetrahydro-5H-pyrido[4′,3′:4,5]pyrrolo[2,3-b]pyridine

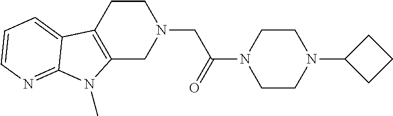

To a solution of 9-methyl-6,7,8,9-tetrahydro-5H-pyrido[4′,3′:4,5]pyrrolo[2,3-b]pyridine (205 mg, 1.09 mmol) in acetonitrile (5 mL) is added NaI (50 mg, 0.333 mmol), K₂CO₃ (451 mg, 3.27 mmol) and 1-(chloroacetyl)-4-cyclobutylpiperazine (283 mg, 1.31 mmol). The mixture is stirred overnight at rt. The mixture is diluted with DCM (25 mL) and filtered through Celite. The filtrate is concentrated and the residue is purified by PTLC eluting with EtOAc/MeOH/TEA (90:10:5) to yield the title compound. ¹H NMR (300 MHz, CDCl₃) δ 8.24 (1H, dd), 7.74 (3H, d), 7.24 (1H, dd), 7.01 (1H, dd), 3.81 (2H, s), 3.71 (3H, s), 3.67 (4H, m), 3.5 (2H, s), 2.93 (2H, t), 2.81 (2H, t), 2.69 (1H, m), 2.32-2.28 (4H, m), 2.03-1.99 (2H, m), 1.88-1.82 (2H, m); 1.74-1.68 (2H, m); MS (+VE) m/z 368.14 (M⁺+1).

### 10. 2-[2-(4-CYCLOBUTYLPIPERAZIN-1-YL)-2-OXOETHYL]-7-METHOXY-2,3,4,5-TETRAHYDRO-1H-PYRIDO[4,3-B]INDOLE (SCHEME 8)

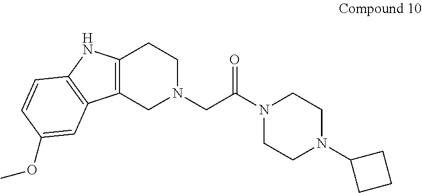

To a solution of 8-methoxy-2,3,4,5-tetrahydro-1H-pyrido[4,3-b]indole (AKos Consulting and Solutions GmbH; 300 mg, 1.48 mmol) in acetonitrile (10 mL) is added NaI (60 mg, 0.40 mmol), K₂CO₃ (300 mg, 2.17 mmol) and 1-(chloroacetyl)-4-cyclobutylpiperazine (321 mg, 1.48 mmol). The mixture is stirred overnight at rt. The mixture is diluted with DCM (25 mL) and filtered through Celite. The filtrate is concentrated and the residue is purified by PTLC eluting with EtOAc/MeOH/TEA (90:10:5) to yield the title compound. ¹H NMR (300 MHz, CDCl₃) δ 7.66 (1H, br ex s), 7.20 (1H, d), 6.93 (1H, d), 6.80 (1H, dd), 3.85 (3H, s), 3.76 (2H, s), 3.71 (4H, m), 3.45 (2H, s), 2.93 (2H, t), 2.79 (2H, t), 2.69 (1H, m), 2.36 (4H, m), 2.03-1.99 (2H, m), 1.88-1.82 (2H, m); 1.74-1.68 (2H, m); MS (+VE) m/z 383.24 (M⁺+1).

### 11. 2-[2-(4-CYCLOBUTYLPIPERAZIN-1-YL)-2-OXOETHYL]-8-METHOXY-1,2,3,4-TETRAHYDROPYRAZINO[1,2-A]INDOLE (SCHEME 9)

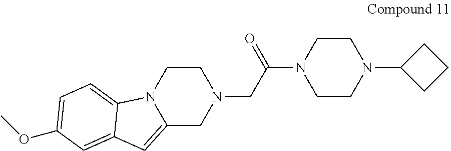

To a solution of 8-methoxy-1,2,3,4-tetrahydropyrazino[1,2-a]indole (ARKIVOC 2004(v): 286-300; 127 mg, 0.628 mmol) in acetonitrile (4 mL) is added NaI (50 mg, 0.33 mmol), K₂CO₃ (300 mg, 2.17 mmol) and 1-(chloroacetyl)-4-cyclobutylpiperazine (150 mg, 0.69 mmol). The mixture is stirred overnight at rt. The mixture is diluted with DCM (25 mL) and filtered through Celite. The filtrate is concentrated and the residue is purified by PTLC eluting with EtOAc/MeOH/TEA (95:5:5) to yield the title compound. ¹H NMR (300 MHz, CDCl₃) δ 7.16 (1H, d), 7.03 (1H, d), 6.82 (1H, dd), 6.13 (1H, s), 4.05 (2H, t), 3.87 (2H, s), 3.84 (3H, s), 3.66 (4H, m), 3.41 (2H, s), 3.04 (2H, t), 2.69 (1H, m), 2.30 (4H, m), 2.03-1.99 (2H, m), 1.88-1.82 (2H, m); 1.74-1.64 (2H, m); MS (+VE) m/z 383.13 (M⁺+1).

### 12. 1-(4-CYCLOBUTYL-PIPERAZIN-1-YL)-2-(1,2-DIHYDRO-4H-3,8A,9-TRIAZA-FLUOREN-3-YL)-ETHANONE (SCHEME 10)

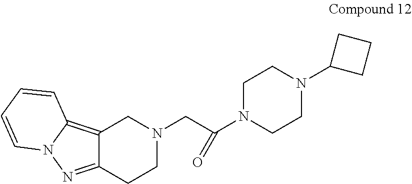

### Step 1. 2-(2-Azido-ethyl)-pyrazolo[1,5-a]pyridine

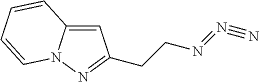

To a solution of 2-pyrazolo[1,5-a]pyridin-2-yl-ethanol (1.0 g, 6.2 mmol) in DCM (30 mL) containing TEA (1.25 g, 12.4 mmol, 2.0 eq.) cooled to 0° C. is added methanesulfonyl chloride (850 mg, 7.4 mmol, 1.2 eq.). The mixture is stirred at 0° C. for an additional 1 h, then warmed to rt and stirred for an additional 3 h. DCM is evaporated, and the residue is dissolved in DMF (10 mL). To the solution is added sodium azide (1.20 g, 18.6 mmol, 3.0 eq.). The reaction mixture is stirred at 50° C. for an additional 24 h. The reaction mixture is cooled to rt, poured into ice-cooled water (20 mL), and extracted with EtOAc (15 mL×3). The combined organic extracts are washed with water and brine, dried over sodium sulfate. The solvent is removed under reduced pressure to yield a residue which is purified through chromatography eluting with hexane/EtOAc (80:20) to give the azido compound. MS (+VE) m/z 189.10 (M⁺+1).

### Step 2. 2-Pyrazolo[1,5-a]pyridin-2-yl-ethylamine

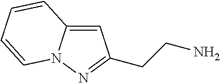

To a solution of 2-(2-azido-ethyl)-pyrazolo[1,5-a]pyridine (470g, 2.5 mmol) in THF (10 mL) cooled to 0° C. is added triphenylphosphine (1.96 g, 7.5 mmol, 3 eq.). The mixture is stirred at 0° C. for an additional 1 h, then warmed to rt and stirred for an additional 3 h. THF is evaporated, and the residue is purified through chromatography eluting with DCM/MeOH (90:10) to give the title compound. MS (+VE) m/z 162.10 (M⁺+1).

### Step 3. 1,2,3,4-Tetrahydro-3,8a,9-triaza-fluorene

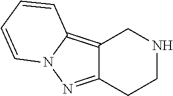

To a solution of 2-pyrazolo[1,5-a]pyridin-2-yl-ethylamine (400 g, 2.47 mmol) in formic acid (3 mL) at rt is added paraformaldehyde (83 mg, 2.59 mmol, 1.05 eq.). The mixture is stirred at rt overnight. Formic acid is evaporated, and the residue is dissolved in DCM (10 mL) and washed with 2.0 N aqueous NaOH solution. The aqueous layer is extracted with DCM twice (2×5 mL). The combined organics are dried over sodium sulfate. The solvent is removed under reduced pressure to yield a residue which is purified through silica gel chromatography eluting with DCM/MeOH (90:10) to give the title compound. MS (+VE) m/z 174.2 (M⁺+1).

### Step 4. 1-(4-Cyclobutyl-piperazin-1-yl)-2-(1,2-dihydro-4H-3,8a,9-triaza-fluoren-3-yl)-ethanone

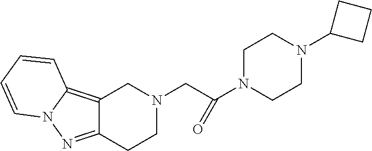

To a stirred solution of 1,2,3,4-tetrahydro-3,8a,9-triaza-fluorene (210 mg, 1.21 mmol) in acetonitrile (10.0 mL) is added 1-(chloroacetyl)-4-cyclobutylpiperazine (288 mg, 1.33 mmol, 1.1 eq.), K₂CO₃ (332 mg, 2.4 mmol, 2.0 eq.), and NaI (50 mg). The resulting mixture is stirred at rt overnight. Water (10.0 mL) is added to quench the reaction, and then the solvent is evaporated. The residue is extracted with DCM (3×10 mL). The combined organic phase is dried over sodium sulfate, and the solvent is removed under reduced pressure to give a residue that is purified by PTLC eluting with EtOAc/TEA (96:4) to give the title compound. ¹H NMR (300 MHz, CDCl₃) δ 8.34 (1H, d) 7.25 (1H, d), 7.02 (1H, dd), 6.40 (1H, dt), 3.76 (2H, s), 3.64 (4H, m), 3.44 (2H, s), 2.84˜3.02 (4H, M), 2.65 (1H, m), 2.26 (4H, m), 1.60˜2.10 (6H, m); MS (+VE) m/z 354.2 (M⁺+1).

### 13. 1-(4-CYCLOBUTYL-PIPERAZIN-1-YL)-2-(3,4-DIHYDRO-1H-BENZO[4,5]FURO[2,3-C]PYRIDIN-2-YL)-ETHANONE (SCHEME 11)

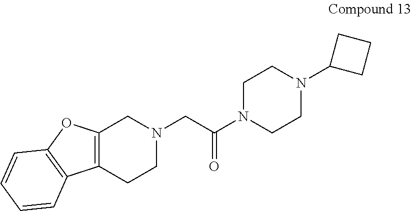

### Step 1. 1,2,3,4-Tetrahydro-benzo[4,5]furo[2,3-c]pyridine

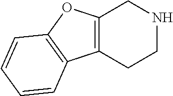

To a solution of 2-benzofuran-3-yl-ethylamine (510 g, 23.16 mmol) in formic acid (3 mL) at rt is added paraformaldehyde (104 mg, 3.17 mmol, 1.1 eq.). The mixture is stirred at 50° C. for 3 h. Formic acid is evaporated. The residue is dissolved in DCM (10 mL) and washed with 2.0 N aqueous NaOH solution. The aqueous layer is extracted with DCM twice (2×5 mL). The combined organics are dried over sodium sulfate. The solvent is removed under reduced pressure to yield a residue which is purified through chromatography eluting with DCM/MeOH (90:10) to give the title compound. MS (+VE) m/z 174.1 (M⁺+1).

### Step 2. 1-(4-Cyclobutyl-piperazin-1-yl)-2-(3,4-dihydro-1H-benzo[4,5]furo[2,3-c]pyridin-2-yl)-ethanone

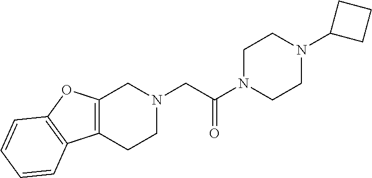

To a stirred solution of 1,2,3,4-tetrahydro-benzo[4,5]furo[2,3-c]pyridine (210 mg, 1.21 mmol) in acetonitrile (10.0 mL) is added 1-(chloroacetyl)-4-cyclobutylpiperazine (288 mg, 1.33 mmol, 1.1 eq.), K₂CO₃ (332 mg, 2.4 mmol, 2.0 eq.), and NaI (50 mg). The resulting mixture is stirred at rt overnight. Water (10.0 mL) is added to quench the reaction, and then the acetonitrile is evaporated. The residue is extracted with DCM (3×10 mL). The combined organic extracts are dried over sodium sulfate, and the solvent is removed under reduced pressure to give a residue that is purified by PTLC eluting with EtOAc/TEA (96:4) to give the title compound. ¹H NMR (400 MHz, CDCl₃) δ 7.40˜7.45 (2H, m), 7.18˜7.24 (2H, dd, m), 3.76 (2H, s), 3.63 (4H, m), 3.46 (2H, s), 2.92 (2H, t), 2.74 (2H, t), 2.32 (5H, m), 1.60˜2.10 (6H, m); MS (+VE) m/z 354.2 (M⁺+1).

### 14. 1-(4-CYCLOBUTYL-PIPERAZIN-1-YL)-2-(3,4-DIHYDRO-1H-BENZO[4,5]THIENO[2,3-C]PYRIDIN-2-YL)-ETHANONE (SCHEME 11)

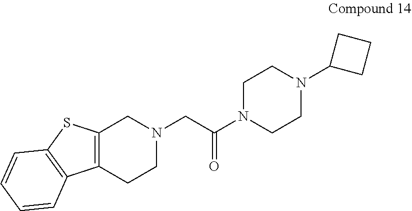

### Step 1. 2-Benzo[b]thiophen-3-yl-ethylamine

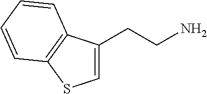

To the solution of benzo[b]thiophen-3-yl-acetonitrile (1.03 g, 5.95 mmol) in THF (20 mL) is added a solution of BH₃ in THF (1.0 N, 3 mL, 13 mmol, 2.2 eq.). The resulting solution is stirred at 50° C. for 16 h. Water (10 mL) is added to quench the reaction and the remaining BH₃ is destroyed with hydrochloric acid (37%, 1.0 mL). The solution is basified with aqueous NaOH solution until the pH>10. THF is evaporated, and the residue is extracted with DCM (3×10 mL). The combined organic extracts are dried over sodium sulfate. The solvent is removed under reduced pressure to yield a residue which is purified through chromatography eluting with DCM/MeOH (90:10) to give the title amine compound. MS (+VE) m/z 178.2 (M⁺+1).

### Step 2. 1,2,3,4-Tetrahydro-benzo[4,5]thieno[2,3-c]pyridine

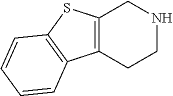

To a solution of 2-benzo[b]thiophen-3-yl-ethylamine (596 mg, 3.36 mmol) in formic acid (4 mL) at rt is added paraformaldehyde (101 mg, 3.17 mmol, 1.0 eq.). The mixture is stirred at 50° C. for 2 h. Formic acid is evaporated, and the residue is dissolved in DCM (10 mL), washed with 2.0 N aqueous NaOH solution. The aqueous layer is extracted with DCM twice (2×5 mL). The combined organics are dried over sodium sulfate. The solvent is removed under reduced pressure to yield a residue which is purified through chromatography eluting with DCM/MeOH (90:10) to give the title compound. MS (+VE) m/z 190.2 (M⁺+1).

### Step 3. 1-(4-cyclobutyl-piperazin-1-yl)-2-(3,4-dihydro-1H-benzo[4,5]thieno[2,3-c]pyridin-2-yl)-ethanone

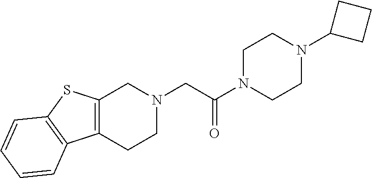

To a stirred solution of 1,2,3,4-tetrahydro-benzo[4,5]thieno[2,3-c]pyridine (190 mg, 1.0 mmol) in acetonitrile (10.0 mL) is added 1-(chloroacetyl)-4-cyclobutylpiperazine (238 mg, 1.10 mmol, 1.1 eq.), K₂CO₃ (332 mg, 2.4 mmol, 2.4 eq.), and NaI (50 mg). The resulting mixture is stirred at rt overnight. Water (10.0 mL) is added to quench the reaction, and then the acetonitrile is evaporated. The residue is extracted with DCM (10 mL×3). The combined organic phase is dried over sodium sulfate, and the solvent is removed under reduced pressure to give a residue that is purified by PTLC eluting with EtOAc/TEA (96:4) to give the title compound. ¹H NMR (300 MHz, CDCl₃) δ 7.78 (1H, d), 7.59 (1H, d), 7.25˜7.40 (2H, m), 3.85 (2H, s), 3.66 (4H, m), 3.45 (2H, s), 2.97 (2H, t), 2.87 (2H, t), 2.69 (1H, m), 2.31 (4H, m), 1.60˜2.10 (6H, m); MS (+VE) m/z 370.1 (M⁺+1).

### 15. 7-[2-(4-CYCLOBUTYL-PIPERAZIN-1-YL)-2-OXO-ETHYL]-3-METHYL-5,6,7,8-TETRAHYDRO-3H-PYRIDO[4′,3′:4,5]THIENO[2,3-D]PYRIMIDIN-4-ONE (SCHEME 12)

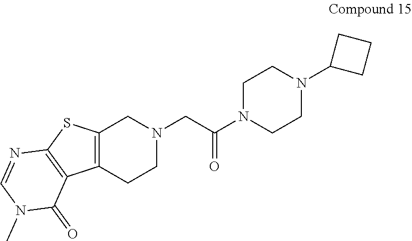

### Step 1. 3-Methyl-4-oxo-3,5,6,8-tetrahydro-4H-pyrido[4′,3′:4,5]thieno[2,3-d]pyrimidine-7-carboxylic acid ethyl ester

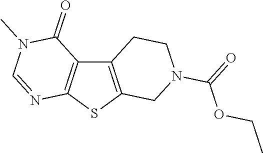

To a solution of 4-oxo-3,5,6,8-tetrahydro-4H-pyrido[4′,3′:4,5]thieno[2,3-d]pyrimidine-7-carboxylic acid ethyl ester (1.40 g, 5.0 mmol) in acetonitrile (30 mL) at 0° C. is added K₂CO₃ (1.38 g, 10 mmol, 2.0 eq.) followed by the addition of iodomethane (851 mg, 6.0 mmol, 1.2 eq.). The mixture is stirred at 50° C. for 5 h. Acetonitrile is evaporated, and the residue is extracted with DCM twice (2×10 mL). The combined organics are washed with brine and dried over sodium sulfate. The solvent is removed under reduced pressure to yield a residue which is purified through chromatography eluting with hexane/EtOAc (70:30) to give the title compound. MS (+VE) m/z 294.2 (M⁺+1).

### Step 2. 3-Methyl-5,6,7,8-tetrahydro-3H-pyrido[4′,3′:4,5]thieno[2,3-d]pyrimidin-4-one

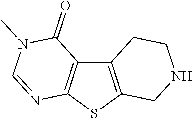

To a solution of 3-methyl-4-oxo-3,5,6,8-tetrahydro-4H-pyrido[4′,3′:4,5]thieno[2,3-d]pyrimidine-7-carboxylic acid ethyl ester (1.18 g, 4.0 mmol) in EtOH (15 mL) is added water (10 mL) followed by the addition of NaOH (800 mg, 20 mmol, 5 eq.). The resulting mixture is refluxed for 24 h. The reaction mixture is cooled to rt, and EtOH is evaporated under reduced pressure. The residue is extracted with DCM (20 mL×2). The combined organics are washed with brine and dried over sodium sulfate. The solvent is removed under reduced pressure to yield a residue which is purified through chromatography eluting with DCM/MeOH (90:10) to give the title compound. MS (+VE) m/z 222.1 (M⁺+1).

### Step 3. 7-[2-(4-Cyclobutyl-piperazin-1-yl)-2-oxo-ethyl]-3-methyl-5,6,7,8-tetrahydro-3H-pyrido[4′,3′:4,5]thieno[2,3-d]pyrimidin-4-one

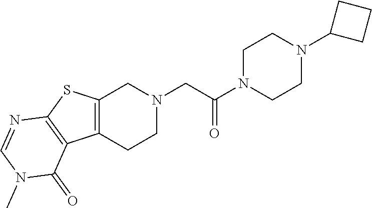

To a stirred solution of 3-methyl-5,6,7,8-tetrahydro-3H-pyrido[4′,3′:4,5]thieno[2,3-d]pyrimidin-4-one (221 mg, 1.0 mmol) in acetonitrile (10.0 mL) is added 1-(chloroacetyl)-4-cyclobutylpiperazine (238 mg, 1.10 mmol, 1.1 eq.), K₂CO₃ (332 mg, 2.4 mmol, 2.4 eq.), and NaI (50 mg). The resulting mixture is stirred at 45° C. overnight. Water (10.0 mL) is added to quench the reaction, and the acetonitrile is evaporated. The residue is extracted with DCM (10 mL×3). The combined organic extracts are dried over sodium sulfate, and the solvent is removed under reduced pressure to give a residue that is purified by PTLC eluting with EtOAc/EtOH/TEA (96:4:4) to give the title compound. ¹H NMR (300 MHz, CDCl₃) δ 7.92 (1H, 3), 3.77 (2H, s), 3.60 (4H, m), 3.56 (3H, s), 3.41 (2H, s), 2.93 (2H, t), 2.88 (2H, t), 2.68 (1H, m), 2.28 (4H, m), 1.60˜2.10 (6H, m); MS (+VE) m/z 402.1 (M⁺+1).

### 16. 1-(4-CYCLOBUTYL-PIPERAZIN-1-YL)-2-(5,8-DIHYDRO-6H-PYRIDO[4′,3′:4,5]-THIENO[2,3-D]PYRIMIDIN-7-YL)-ETHANONE (SCHEME 12)

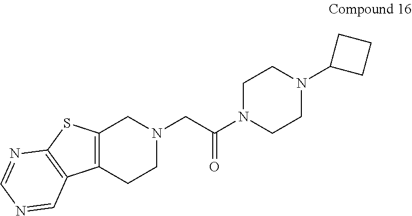

### Step 1. 4-Chloro-5,8-dihydro-6H-pyrido[4′,3′:4,5]thieno[2,3-d]pyrimidine-7-carboxylic acid ethyl ester

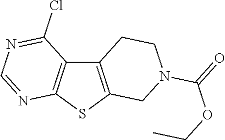

To a mixture of 4-oxo-3,5,6,8-tetrahydro-4H-pyrido[4′,3′:4,5]thieno[2,3-d]pyrimidine-7-carboxylic acid ethyl ester (1.40 g, 5.0 mmol) and pyridine (791 mg) is added POCl₃ (3.06 g, 4.0 eq.). The mixture is stirred at 100° C. for 3 h. POCl₃ is evaporated, and the residue is taken in EtOAc (30 mL), basified with aqueous NaHCO₃ solution. The organic layer is collected, washed with brine, dried over sodium sulfate. The solvent is removed under reduced pressure to yield a residue which is purified through chromatography eluting with hexane/EtOAC (70:30) to give the title compound. MS (+VE) m/z 298.2(M⁺+1).

### Step 2. 5,8-Dihydro-6H-pyrido[4′,3′:4,5]thieno[2,3-d]pyrimidine-7-carboxylic acid ethyl ester

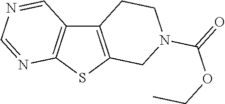

To a mixture of 4-chloro-5,8-dihydro-6H-pyrido[4′,3′:4,5]thieno[2,3-d]pyrimidine-7-carboxylic acid ethyl ester (1.00 g, 3.34 mmol) and MeOH (10 mL) in a 100 mL round bottom flask is added palladium on charcoal (10%, 100 mg). The mixture is hydrogenated with a hydrogen balloon at rt for 3 h. Palladium on charcoal is filtered off through Celite, and the filter cake is washed with MeOH (5 mL×2). The combined organic washes are concentrated under reduced pressure, and the residue is taken up in EtOAc (30 mL). The solution is basified with aqueous NaHCO₃ solution. The organic layer is collected, washed with brine, and dried over sodium sulfate. The solvent is removed under reduced pressure to yield a residue which is purified through chromatography eluting with hexane/EtOAC (70:30) to give the desired compound. MS (+VE) m/z 264.1 (M⁺+1).

### Step 3. 5,6,7,8-Tetrahydro-pyrido[4′,3′:4,5]thieno[2,3-d]pyrimidine

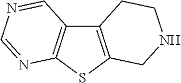

To a solution of 5,8-dihydro-6H-pyrido[4′,3′:4,5]thieno[2,3-d]pyrimidine-7-carboxylic acid ethyl ester (800g, 3.0 mmol) in EtOH (15 mL) is added water (10 mL) followed by the addition of NaOH (600 mg, 15 mmol, 5 eq.). The resulting mixture is refluxed for 24 h. The reaction mixture is cooled to rt, and EtOH is evaporated under reduced pressure. The residue is extracted with DCM (2×20 mL), and washed with brine. The combined organics are dried over sodium sulfate. The solvent is removed under reduced pressure to yield a residue which is purified through chromatography eluting with DCM/MeOH (90:10) to give the title compound. MS (+VE) m/z 192.2 (M⁺+1).

### Step 4. 1-(4-Cyclobutyl-piperazin-1-yl)-2-(5,8-dihydro-6H-pyrido[4′,3′:4,5]thieno[2,3-d]pyrimidin-7-yl)-ethanone

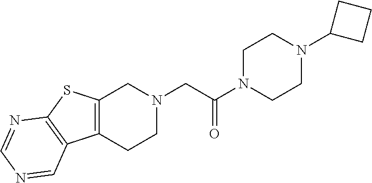

To a stirred solution of 5,6,7,8-tetrahydro-pyrido[4′,3′:4,5]thieno[2,3-d]pyrimidine (191 mg, 1.0 mmol) in acetonitrile (10.0 mL) is added 1-(chloroacetyl)-4-cyclobutylpiperazine (238 mg, 1.10 mmol, 1.1 eq.), K₂CO₃ (332 mg, 2.4 mmol, 2.4 eq.), and NaI (50 mg). The resulting mixture is stirred at 45° C. overnight. Water (10.0 mL) is added to quench the reaction, and then the acetonitrile is evaporated. The residue is extracted with DCM (3×10 mL). The combined organic extracts are dried over sodium sulfate, and the solvent is removed under reduced pressure to give a residue that is purified by PTLC eluting with EtOAc/EtOH/TEA (96:4:4) to give the title compound. ¹H NMR (300 MHz, CDCl₃) δ 9.03 (1H, s), 8.91 (1H, s), 3.90 (2H, s), 3.62 (4H, m), 3.48 (2H, s), 3.01 (2H, t), 2.92 (2H, m), 2.69 (1H, m), 2.30 (4H, m), 1.60˜2.10 (6H, m); MS (+VE) m/z 372.2 (M⁺+1).

### 17. 1-(4-CYCLOBUTYL-PIPERAZIN-1-YL)-2-(2-METHYL-5,8-DIHYDRO-6H-PYRIDO-[4′,3′:4,5]THIENO[2,3-D]PYRIMIDIN-7-YL)-ETHANONE (SCHEME 12)

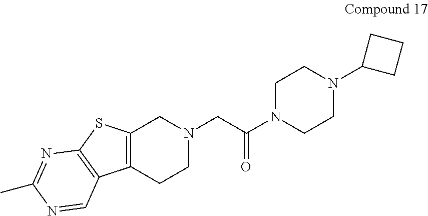

### Step 1. 4-Hydroxy-2-methyl-5,8-dihydro-6H-pyrido[4′,3′:4,5]thieno[2,3-d]pyrimidine-7-carboxylic acid ethyl ester

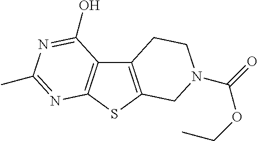

To a solution of 2-amino-4,7-dihydro-5H-thieno[2,3-c]pyridine-3,6-dicarboxylic acid diethyl ester (2.98 g, 10.0 mmol) in HCl-dioxane (4.0 N, 20 mL) is added CH₃CN (2.0 mL). The mixture is stirred at rt for 8 h, heated to 100° C., and stirred for an additional 8 h at 100° C. Dioxane is evaporated, and the residue is taken up in water (30 mL). The solution is basified with aqueous NaHCO₃ solution. The resulting solid is collected by filtration, washed with water, dried under high vacuum to give the title compound. MS (+VE) m/z 293.1(M⁺+1).

### Step 2. 4-Chloro-2-methyl-5,8-dihydro-6H-pyrido[4′,3′:4,5]thieno[2,3-d]pyrimidine-7-carboxylic acid ethyl ester

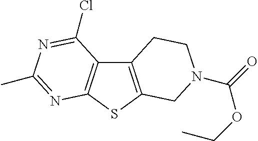

To a mixture of 4-hydroxy-2-methyl-5,8-dihydro-6H-pyrido[4′,3′:4,5]thieno[2,3-d]pyrimidine-7-carboxylic acid ethyl ester (1.47 g, 5.0 mmol) and pyridine (791 mg) is added POCl₃ (3.06 g, 4.0 eq.). The mixture is stirred at 100° C. for 3 h. Excess POCl₃ is evaporated, and the residue is taken up in EtOAc (30 mL). The solution is basified with aqueous NaHCO₃ solution. The organic layer is collected, washed with brine, and dried over sodium sulfate. The solvent is removed under reduced pressure to yield a residue which is purified through chromatography eluting with hexane/EtOAc (80:20) to give the title compound. MS (+VE) m/z 312.0(M⁺+1).

### Step 3. 2-Methyl-5,8-dihydro-6H-pyrido[4′,3′:4,5]thieno[2,3-d]pyrimidine-7-carboxylic acid ethyl ester

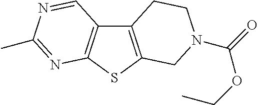

To a mixture of 4-chloro-2-methyl-5,8-dihydro-6H-pyrido[4′,3′:4,5]thieno[2,3-d]pyrimidine-7-carboxylic acid ethyl ester (1.00 g, 3.21 mmol) and MeOH (10 mL) in a 100 mL round bottom flask is added palladium on charcoal (10%, 100 mg). The mixture is hydrogenated with hydrogen filled balloon at rt for 3 h. Palladium on charcoal is filtered off through Celite, and the filter cake is washed with MeOH (5 mL×2). The combined organic washes are concentrated under reduced pressure, and the residue is taken up in EtOAc (30 mL). The solution is basified with aqueous NaHCO₃ solution. The organic layer is collected, washed with brine, and dried over sodium sulfate. The solvent is removed under reduced pressure to yield a residue which is purified through chromatography eluting with hexane/EtOAc (70:30) to give the title compound. MS (+VE) m/z 278.10 (M⁺+1).

### Step 4. 2-Methyl-5,6,7,8-tetrahydro-pyrido[4′,3′:4,5]thieno[2,3-d]pyrimidine

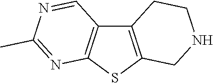

To the solution of 2-methyl-5,8-dihydro-6H-pyrido[4′,3′:4,5]thieno[2,3-d]pyrimidine-7-carboxylic acid ethyl ester (800 g, 2.88 mmol) in EtOH (15 mL) is added water (10 mL), followed by the addition of NaOH (600 mg, 15 mmol, 5.2 eq.). The resulting mixture is refluxed for 24 h. The reaction mixture is cooled to rt, and the EtOH is evaporated under reduced pressure. The residue is extracted with DCM (2×20 mL). The combined organics are washed with brine and dried over sodium sulfate. The solvent is removed under reduced pressure to yield a residue which is purified through chromatography eluting with DCM/MeOH (90:10) to give the title compound. MS (+VE) m/z 206.1 (M⁺+1).

### Step 5. 1-(4-Cyclobutyl-piperazin-1-yl)-2-(2-methyl-5,8-dihydro-6H-pyrido[4′,3′:4,5]thieno[2,3-d]pyrimidin-7-yl)-ethanone

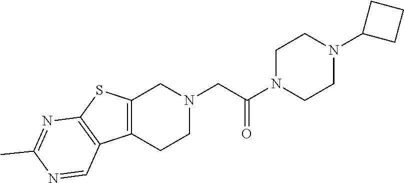

To a stirred solution of 2-methyl-5,6,7,8-tetrahydro-pyrido[4′,3′:4,5]thieno[2,3-d]pyrimidine (205 mg, 1. 0 mmol) in acetonitrile (10.0 mL) is added 1-(chloroacetyl)-4-cyclobutylpiperazine (238 mg, 1.10 mmol, 1.1 eq.), K₂CO₃ (332 mg, 2.4 mmol, 2.4 eq.), and NaI (50 mg). The resulting mixture is stirred at 45° C. overnight. Water (10.0 mL) is added to quench the reaction, and the acetonitrile is evaporated. The residue is extracted with DCM (3×10 mL). The combined organic extracts are dried over sodium sulfate, and the solvent is removed under reduced pressure to give a residue that is purified by PTLC eluting with EtOAc/EtOH/TEA (96:4:4) to give the title compound. ¹H NMR (300 MHz, CDCl₃) δ 8.81 (1H, s), 3.87 (2H, s), 3.62 (4H, m), 3.47 (2H, s), 3.00 (2H, t), 2.91 (2H, m), 2.82 (3H, s), 2.70 (1H, m), 2.30 (4H, m), 1.60˜2.10 (6H, m); MS (+VE) m/z 386.20 (M⁺+1).

### Example 2

### Preparation of Additional Representative Compounds

Using routine modifications, the starting materials may be varied and additional steps employed to produce other compounds provided herein. Compounds listed in Tables I and II are prepared using such methods. A “*” in the column headed “Kᵢ” in Table I indicates that the compound has a Kᵢ in the assay of Example 7 that is less than 1 micromolar. A “*” in the column headed “%” in Table II indicates that the percent inhibition of agonist-induced GTP-gammaS binding to H3, in the 4 μM screening assay described in Example 8, is at least 90%.

The molecular weight (presented as M+1) obtained using the method described above is shown in the column headed “MS.”

TABLE I

| | Compound | Name | MS | Kᵢ |
|---|---|---|---|---|
| 18 | 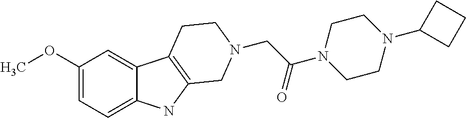 | 2-[2-(4- Cyclobutylpiperazin-1- yl)-2-oxoethyl]-6- methoxy-2,3,4,9- tetrahydro-1H-beta- carboline | 383.21 | * |
| 19 | 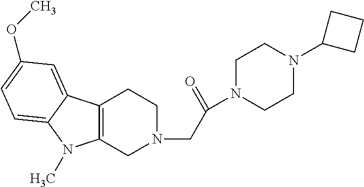 | 2-[2-(4- Cyclobutylpiperazin-1- yl)-2-oxoethyl]-6- methoxy-9-methyl- 2,3,4,9-tetrahydro-1H- beta-carboline | 397.22 | * |
| 20 | 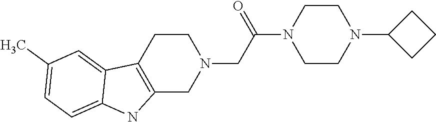 | 2-[2-(4- Cyclobutylpiperazin-1- yl)-2-oxoethyl]-6- methyl-2,3,4,9- tetrahydro-1H-beta- carboline | 367.29 | * |
| 21 | 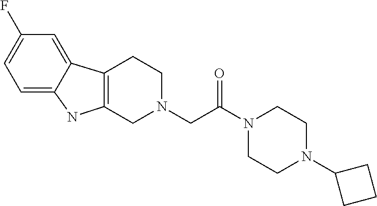 | 2-[2-(4- Cyclobutylpiperazin-1- yl)-2-oxoethyl]-6- fluoro-2,3,4,9- tetrahydro-1H-beta- carboline | 371.32 | * |
| 22 | 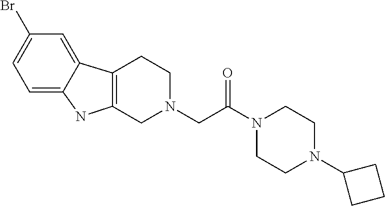 | 6-Bromo-2-[2-(4- cyclobutylpiperazin-1- yl)-2-oxoethyl]-2,3,4,9- tetrahydro-1H-beta- carboline | 433.23 | * |
| 23 | 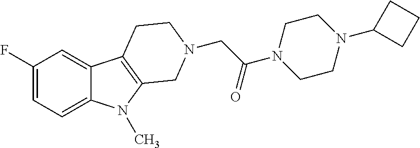 | 2-[2-(4- Cyclobutylpiperazin-1- yl)-2-oxoethyl]-6- fluoro-9-methyl-2,3,4,9- tetrahydro-1H-beta- carboline | 385.33 | * |
| 24 | 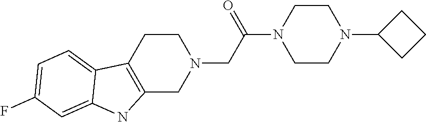 | 2-[2-(4- Cyclobutylpiperazin-1- yl)-2-oxoethyl]-7- fluoro-2,3,4,9- tetrahydro-1H-beta- carboline | 371.35 | * |
| 25 | 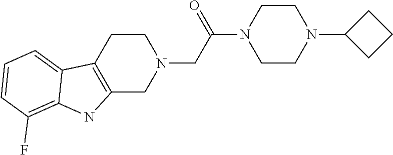 | 2-[2-(4- Cyclobutylpiperazin-1- yl)-2-oxoethyl]-8- fluoro-2,3,4,9- tetrahydro-1H-beta- carboline | 371.30 | * |
| 26 | 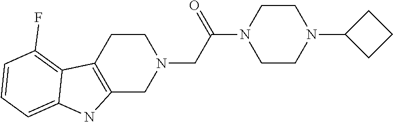 | 2-[2-(4- Cyclobutylpiperazin-1- yl)-2-oxoethyl]-5- fluoro-2,3,4,9- tetrahydro-1H-beta- carboline | 371.30 | * |
| 27 | 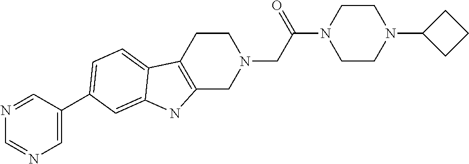 | 2-[2-(4- Cyclobutylpiperazin-1- yl)-2-oxoethyl]-7- pyrimidin-5-yl-2,3,4,9- tetrahydro-1H-beta- carboline | 431.35 | * |
| 28 | 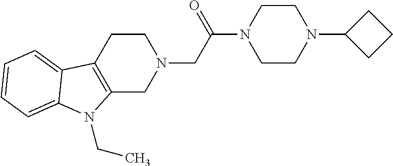 | 2-[2-(4- Cyclobutylpiperazin-1- yl)-2-oxoethyl]-9-ethyl- 2,3,4,9-tetrahydro-1H- beta-carboline | 425.31 | * |
| 29 | 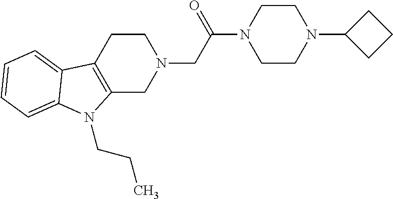 | 2-[2-(4- Cyclobutylpiperazin-1- yl)-2-oxoethyl]-9- propyl-2,3,4,9- tetrahydro-1H-beta- carboline | 395.38 | * |
| 30 | 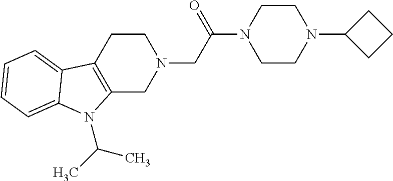 | 2-[2-(4- Cyclobutylpiperazin-1- yl)-2-oxoethyl]-9- isopropyl-2,3,4,9- tetrahydro-1H-beta- carboline | 395.38 | * |
| 31 | 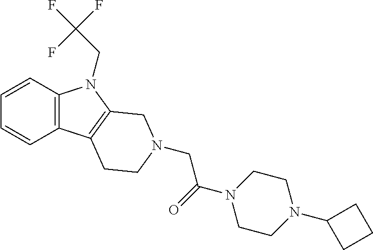 | 2-[2-(4- Cyclobutylpiperazin-1- yl)-2-oxoethyl]-9- (2,2,2-trifluoroethyl)- 2,3,4,9-tetrahydro-1H- beta-carboline | 435.33 | * |
| 32 | 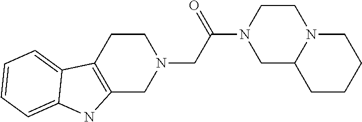 | 2-[2-(Octahydro-2H- pyrido[1,2-a]pyrazin-2- yl)-2-oxoethyl]-2,3,4,9- tetrahydro-1H-beta- carboline | 353.35 | * |
| 33 | 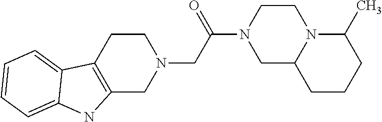 | 2-[2-(6- Methyloctahydro-2H- pyrido[1,2-a]pyrazin-2- yl)-2-oxoethyl]-2,3,4,9- tetrahydro-1571H-beta- carboline | 367.35 | * |

TABLE II

| | Compound | Name | MS | % |
|---|---|---|---|---|
| 34 | 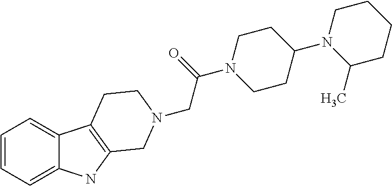 | 2-[2-(2-Methyl-1,4′- bipiperidin-1′-yl)-2- oxoethyl]-2,3,4,9- tetrahydro-1H-beta- carboline | 395.28 | * |
| 35 | 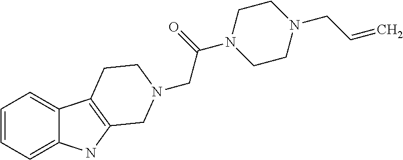 | 2-[2-(4-Allylpiperazin- 1-yl)-2-oxoethyl]- 2,3,4,9-tetrahydro-1H- beta-carboline | 339.23 | * |
| 36 | 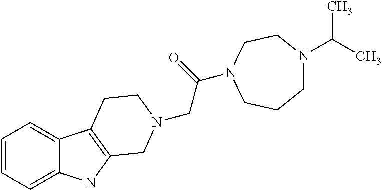 | 2-[2-(4-Isopropyl-1,4- diazepan-1-yl)-2- oxoethyl]-2,3,4,9- tetrahydro-1H-beta- carboline | 355.26 | * |
| 37 | 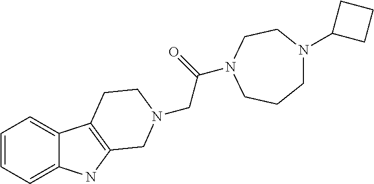 | 2-[2-(4-Cyclobutyl-1,4- diazepan-1-yl)-2- oxoethyl]-2,3,4,9- tetrahydro- 1H-beta-carboline | 367.26 | * |
| 38 | 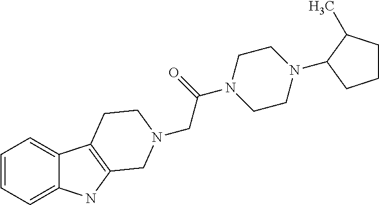 | 2-{2-[4-(2- Methylcyclopentyl)piperazin- l-yl]-2-oxoethyl}- 2,3,4,9-tetrahydro-1H- beta-carboline | 381.29 | * |
| 39 | 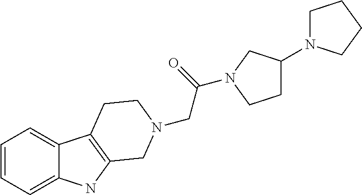 | 2-[2-(1,3′-Bipyrrolidin- 1′-yl)-2-oxoethyl]- 2,3,4,9-tetrahydro-1H- beta-carboline | 353.26 | * |
| 40 | 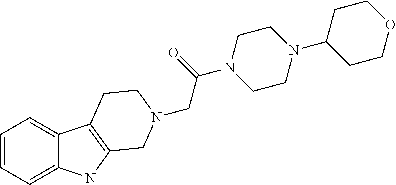 | 2-{2-Oxo-2-[4- (tetrahydro-2H-pyran-4- yl)piperazin-1- yl]ethyl}-2,3,4,9- tetrahydro-1H-beta- carboline | 383.27 | * |
| 41 | 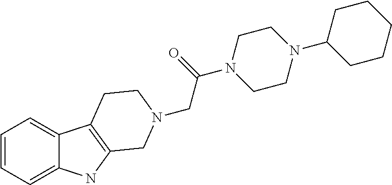 | 2-[2-(4- Cyclohexylpiperazin-1- yl)-2-oxoethyl]-2,3,4,9- tetrahydro-1H-beta- carboline | 381.29 | * |
| 42 | 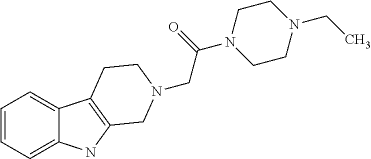 | 2-[2-(4-Ethylpiperazin- 1-yl)-2-oxoethyl]- 2,3,4,9-tetrahydro-1H- beta-carboline | 327.23 | * |
| 43 | 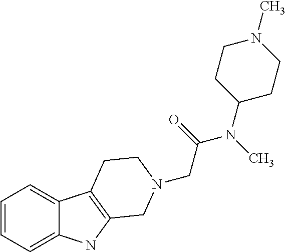 | N-Methyl-N-(1- ethylpiperidin-4-yl)-2- (1,3,4,9-tetrahydro-2H- beta-carbolin-2- yl)acetamide | 341.25 | * |
| 44 | 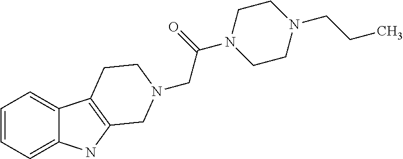 | 2-[2-(4-propylpiperazin- 1-yl)-2-oxoethyl]- 2,3,4,9-tetrahydro-1H- beta-carboline | 341.25 | * |
| 45 | 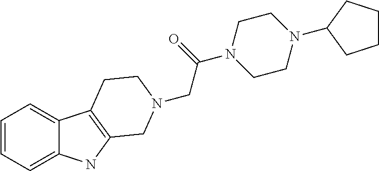 | 2-[2-(4- Cyclopentylpiperazin-1- yl)-2-oxoethyl]-2,3,4,9- tetrahydro-1H-beta- carboline | 367.28 | * |
| 46 | 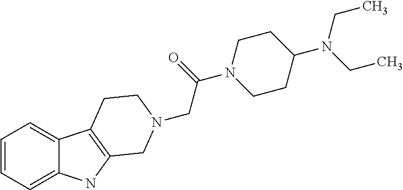 | N,N-Diethyl-1-(1,3,4,9- tetrahydro-2H-beta- carbolin-2- ylacetyl)piperidin-4- amine | 369.2783 | * |
| 47 | | 2-[2-(4-Butyl-1,4- diazepan-1-yl)-2- oxoethyl]-2,3,4,9- tetrahydro-1H-beta- carboline | 369.29 | * |
| 48 | 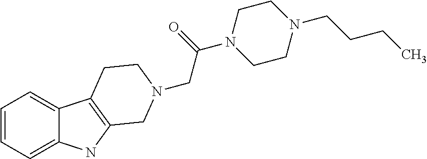 | 2-[2-(4-Butylpiperazin- 1-yl)-2-oxoethyl]- 2,3,4,9-tetrahydro-1H- beta-carboline | 355.27 | * |
| 49 | 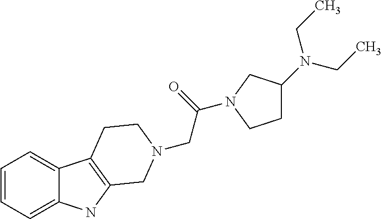 | N,N-Diethyl-1-(1,3,4,9- tetrahydro-2H-beta- carbolin-2- ylacetyl)pyrrolidin-3- amine | 355.27 | * |
| 50 | 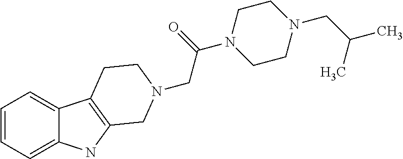 | 2-[2-(4- Isobutylpiperazin-1-yl)- 2-oxoethyl]-2,3,4,9- tetrahydro-1H-beta- carboline | 355.28 | * |
| 51 | 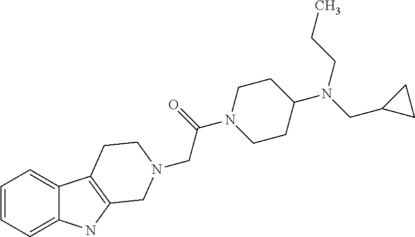 | N-(Cyclopropylmethyl)- N-propyl-1-(1,3,4,9- tetrahydro-2H-beta- carbolin-2-ylacetyl)piperidin- 4-amine | 409.31 | * |
| 52 | 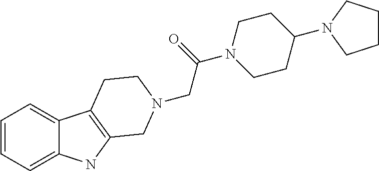 | 2-[2-Oxo-2-(4- pyrrolidin-1- ylpiperidin-1-yl)ethyl]- 2,3,4,9-tetrahydro-1H- beta-carboline | 367.27 | * |
| 53 | 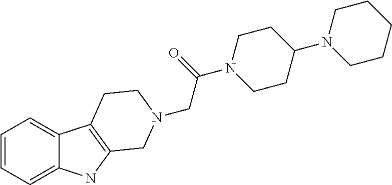 | 2-[2-(1,4′-Bipiperidin- 1′-yl)-2-oxoethyl]- 2,3,4,9-tetrahydro-1H- beta-carboline | 381.29 | * |
| 54 | 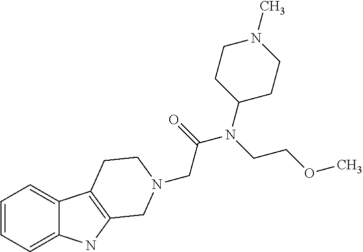 | N-(2-Methoxyethyl)-N- (1-methylpiperidin-4- yl)-2-(1,3,4,9- tetrahydro-2H-beta- carbolin-2-yl)acetamide | 385.28 | * |
| 55 | | 2-{2-Oxo-2-[4-(3- pyrrolidin-1- ylpropyl)piperazin-1- yl]ethyl}-2,3,4,9- tetrahydro-1H-beta- carboline | 410.30 | * |
| 56 | | 2-{2-Oxo-2-[4-(2- piperidin-1- ylethyl)piperazin-1- yl]ethyl}-2,3,4,9- tetrahydro-1H-beta- carboline | 410.30 | * |
| 57 | | 2-{2-Oxo-2-[4-(3- piperidin-1- ylpropyl)piperazin-1- yl]ethyl}-2,3,4,9- tetrahydro-1H-beta- carboline | 424.31 | * |
| 58 | | 2-{2-Oxo-2-[4-(2- pyrrolidin-1- ylethyl)piperazin-1- yl]ethyl}-2,3,4,9- tetrahydro-1H-beta- carboline | 396.29 | * |
| 59 | 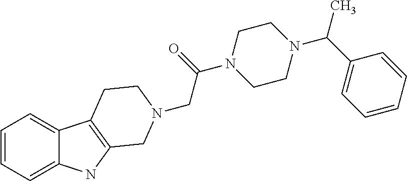 | 2-{2-Oxo-2-[4-(1- phenylethyl)piperazin- 1-yl]ethyl}-2,3,4,9- tetrahydro-1H-beta- carboline | 403.31 | * |
| 60 | 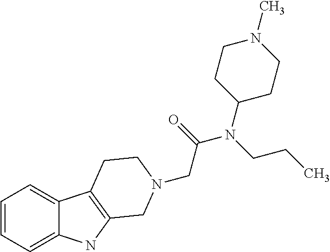 | N-(1-Methylpiperidin- 4-yl)-N-propyl-2- (1,3,4,9-tetrahydro-2H- beta-carbolin-2- yl)acetamide | 369.34 | * |
| 61 | 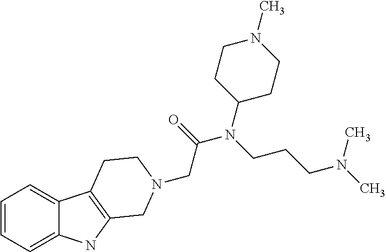 | N-[3- (Dimethylamino)propyl]- N-(1-methylpiperidin- 4-yl)-2-(1,3,4,9- tetrahydro-2H-beta- carbolin-2- yl)acetamide | 412.40 | * |
| 62 | 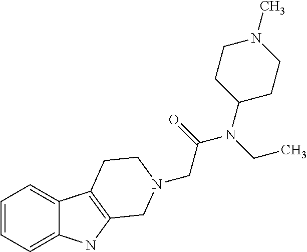 | N-(1-Methylpiperidin- 4-yl)-N-ethyl-2- (1,3,4,9-tetrahydro-2H- beta-carbolin-2- yl)acetamide | 355.34 | * |
| 63 | 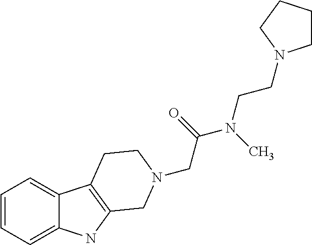 | N-[2- (Dimethylamino)ethyl]- N-methyl-2-(1,3,4,9- tetrahydro-2H-beta- carbolin-2- yl)acetamide | 341.34 | * |

### Example 3

### Preparation of Additional Representative Compounds

Using routine modifications, the starting materials may be varied and additional steps employed to produce other compounds provided herein.

TABLE III

| | Compound | Name |
|---|---|---|
| 64 | 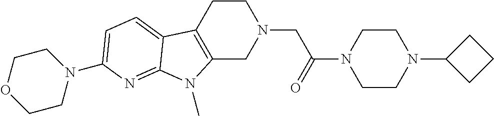 | 7-[2-(4-Cyclobutylpiperazin-1-yl)-2- oxoethyl]-9-methyl-2-morpholin-4-yl- 6,7,8,9-tetrahydro-5H- pyrido[4′,3′:4,5]pyrrolo[2,3-b]pyridine |
| 65 | 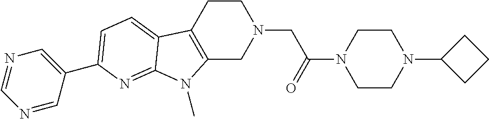 | 7-[2-(4-Cyclobutylpiperazin-1-yl)-2- oxoethyl]-9-methyl-2-pyrimidin-5-yl- 6,7,8,9-tetrahydro-5H- pyrido[4′,3′:4,5]pyrrolo[2,3-b]pyridine |
| 66 | 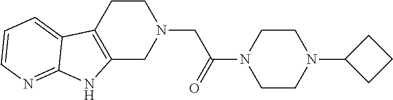 | 7-[2-(4-Cyclobutylpiperazin-1-yl)- 2-oxoethyl]-6,7,8,9-tetrahydro-5H- pyrido[4′,3′:4,5]pyrrolo[2,3-b]pyridine |
| 67 | 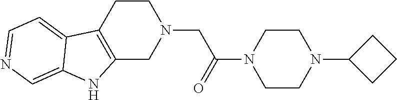 | 2-[2-(4-Cyclobutylpiperazin-1-yl)-2- oxoethyl]-2,3,4,9-tetrahydro-1H- pyrido[4′,3′:4,5]pyrrolo[2,3-c]pyridine |
| 68 | 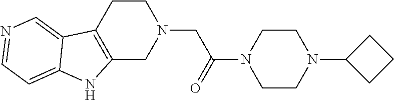 | 7-[2-(4-Cyclobutylpiperazin-1-yl)-2- oxoethyl]-6,7,8,9-tetrahydro-5H- pyrido[3′,4′:4,5]pyrrolo[2,3-c]pyridine |
| 69 | 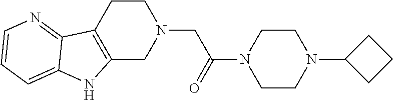 | 7-[2-(4-Cyclobutylpiperazin-1-yl)-2- oxoethyl]-6,7,8,9-tetrahydro-5H- pyrido[4′,3′:4,5]pyrrolo[3,2-b]pyridine |
| 70 | 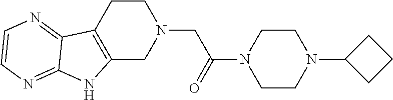 | 7-[2-(4-Cyclobutylpiperazin-1-yl)-2- oxoethyl]-6,7,8,9-tetrahydro-5H- pyrido[4′,3′:4,5]pyrrolo[2,3-b]pyrazine |
| 71 | 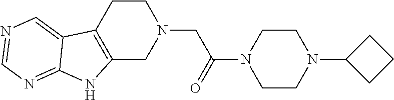 | 7-[2-(4-Cyclobutylpiperazin-1-yl)-2- oxoethyl]-6,7,8,9-tetrahydro-5H- pyrido[4′,3′:4,5]pyrrolo[2,3- d]pyrimidine |
| 72 | 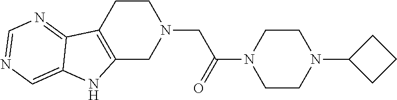 | 7-[2-(4-Cyclobutylpiperazin-1-yl)-2- oxoethyl]-6,7,8,9-tetrahydro-5H- pyrido[4′,3′:4,5]pyrrolo[3,2- d]pyrimidine |
| 73 | 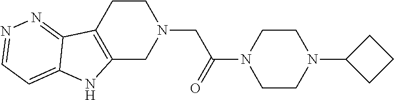 | 7-[2-(4-Cyclobutylpiperazin-1-yl)-2- oxoethyl]-6,7,8,9-tetrahydro-5H- pyrido[4′,3′:4,5]pyrrolo[3,2- c]pyridazine |
| 74 | 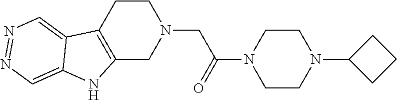 | 7-[2-(4-Cyclobutylpiperazin-1-yl)-2- oxoethyl]-6,7,8,9-tetrahydro-5H- pyrido[4′,3′:4,5]pyrrolo[2,3- d]pyridazine |
| 75 | 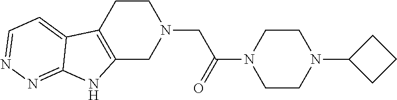 | 7-[2-(4-Cyclobutylpiperazin-1-yl)-2- oxoethyl]-6,7,8,9-tetrahydro-5H- pyrido[4′,3′:4,5]pyrrolo[2,3- c]pyridazine |
| 76 | 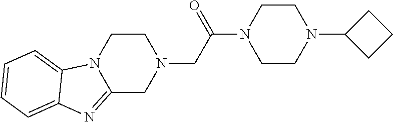 | 2-[2-(4-Cyclobutylpiperazin-1-yl)-2- oxoethyl]-1,2,3,4- tetrahydropyrazino[1,2- a]benzimidazole |
| 77 | 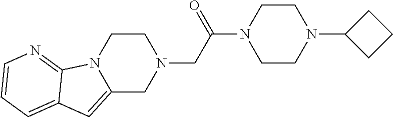 | 7-[2-(4-Cyclobutylpiperazin-1-yl)-2- oxoethyl]-6,7,8,9- tetrahydropyrido[3′,2′:4,5]pyrrolo[1,2- a]pyrazine |
| 78 | 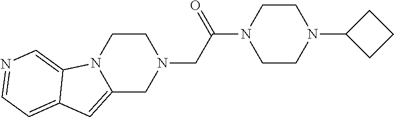 | 2-[2-(4-Cyclobutylpiperazin-1-yl)-2- oxoethyl]-1,2,3,4- tetrahydropyrido[4′,3′:4,5]pyrrolo[1,2- a]pyrazine |
| 79 | 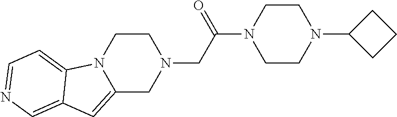 | 8-[2-(4-Cyclobutylpiperazin-1-yl)-2- oxoethyl]-6,7,8,9- tetrahydropyrido[3′,4′:4,5]pyrrolo[1,2- a]pyrazine |
| 80 | 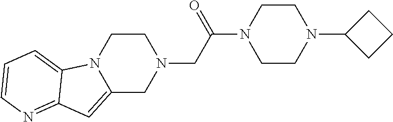 | 8-[2-(4-Cyclobutylpiperazin-1-yl)-2- oxoethyl]-6,7,8,9- tetrahydropyrido[2′,3′:4,5]pyrrolo[1,2- a]pyrazine |
| 81 | 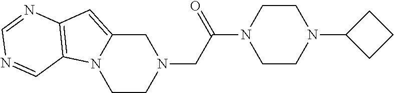 | 8-[2-(4-Cyclobutylpiperazin-1-yl)-2- oxoethyl]-6,7,8,9- tetrahydropyrazino[1′,2′:1,5]pyrrolo[3, 2-d]pyrimidine |
| 82 | 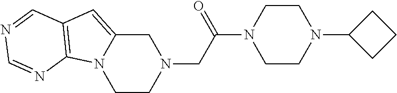 | 7-[2-(4-Cyclobutylpiperazin-1-yl)-2- oxoethyl]-6,7,8,9- tetrahydropyrazino[1′,2′:1,5]pyrrolo[2, 3-d]pyrimidine |
| 83 | 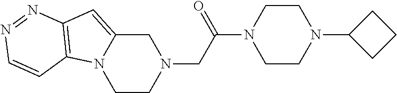 | 8-[2-(4-Cyclobutylpiperazin-1-yl)-2- oxoethyl]-6,7,8,9- tetrahydropyrazino[1′,2′:1,5]pyrrolo[3, 2-c]pyridazine |
| 84 | 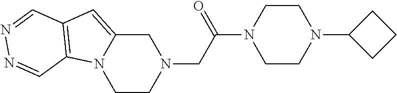 | 8-[2-(4-Cyclobutylpiperazin-1-yl)-2- oxoethyl]-6,7,8,9- tetrahydropyrazino[1′,2′:1,5]pyrrolo[2, 3-d]pyridazine |
| 85 | 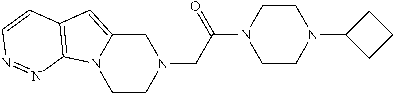 | 7-[2-(4-Cyclobutylpiperazin-1-yl)-2- oxoethyl]-6,7,8,9- tetrahydropyrazino[1′,2′:1,5]pyrrolo[2, 3-c]pyridazine |
| 86 | 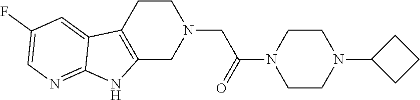 | 7-[2-(4-Cyclobutylpiperazin-1-yl)-2- oxoethyl]-3-fluoro-6,7,8,9-tetrahydro- 5H-pyrido[4′,3′:4,5]pyrrolo[2,3- b]pyridine |
| 87 | 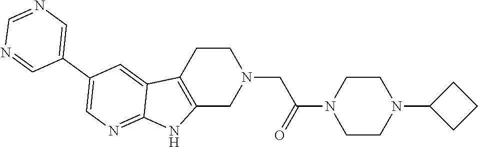 | 7-[2-(4-Cyclobutylpiperazin-1-yl)-2- oxoethyl]-3-pyrimidin-5-yl-6,7,8,9- tetrahydro-5H- pyrido[4′,3′:4,5]pyrrolo[2,3-b]pyridine |
| 88 | 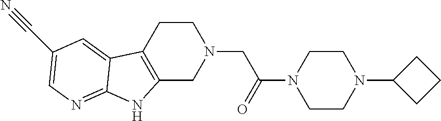 | 7-[2-(4-Cyclobutylpiperazin-1-yl)-2- oxoethyl]-6,7,8,9-tetrahydro-5H- pyrido[4′,3′:4,5]pyrrolo[2,3-b]pyridine- 3-carbonitrile |
| 89 | 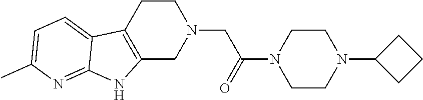 | 7-[2-(4-Cyclobutylpiperazin-1-yl)-2- oxoethyl]-2-methyl-6,7,8,9-tetrahydro- 5H-pyrido[4′,3′:4,5]pyrrolo[2,3- b]pyridine |
| 90 | 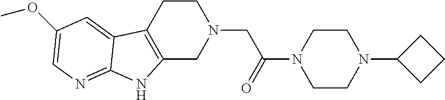 | 7-[2-(4-Cyclobutylpiperazin-1-yl)-2- oxoethyl]-3-methoxy-6,7,8,9- tetrahydro-5H-pyrido[4′,3′:4,5] pyrrolo[2,3-b]pyridine |
| 91 | 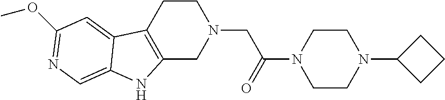 | 2-[2-(4-Cyclobutylpiperazin-1-yl)-2- oxoethyl]-6-methoxy-2,3,4,9- tetrahydro-1H-pyrido[4′,3′:4,5] pyrrolo[2,3-c]pyridine |
| 92 | 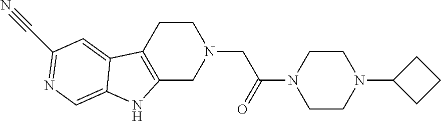 | 2-[2-(4-Cyclobutylpiperazin-1-yl)-2- oxoethyl]-2,3,4,9-tetrahydro-1H- pyrido[4′,3′:4,5]pyrrolo[2,3-c]pyridine- 6-carbonitrile |
| 93 | 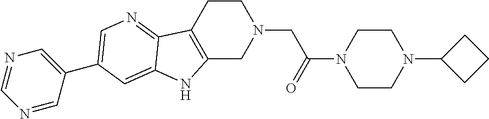 | 7-[2-(4-Cyclobutylpiperazin-1-yl)-2- oxoethyl]-3-pyrimidin-5-yl-6,7,8,9- tetrahydro-5H- pyrido[4′,3′:4,5]pyrrolo[3,2-b]pyridine |
| 94 | 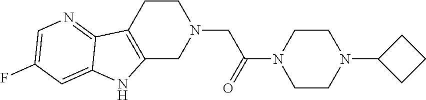 | 7-[2-(4-Cyclobutylpiperazin-1-yl)-2- oxoethyl]-3-fluoro-6,7,8,9-tetrahydro- 5H-pyrido[4′,3′:4,5]pyrrolo[3,2- b]pyridine |
| 95 | 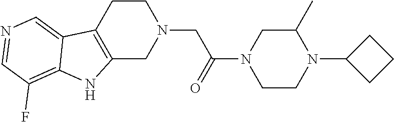 | 7-[2-(4-Cyclobutyl-3-methylpiperazin- 1-yl)-2-oxoethyl]-4-fluoro-6,7,8,9- tetrahydro-5H- pyrido[3′,4′:4,5]pyrrolo[2,3-c]pyridine |
| 96 | 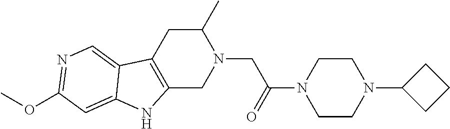 | 7-[2-(4-Cyclobutylpiperazin-1-yl)-2- oxoethyl]-3-methoxy-8-methyl-6,7,8,9- tetrahydro-5H- pyrido[3′,4′:4,5]pyrrolo[2,3-c]pyridine |
| 97 | 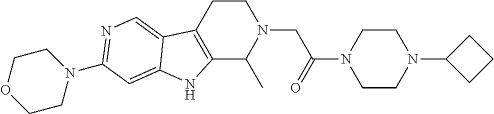 | 7-[2-(4-Cyclobutylpiperazin-1-yl)-2- oxoethyl]-6-methyl-3-morpholin-4-yl- 6,7,8,9-tetrahydro-5H- pyrido[3′,4′:4,5]pyrrolo[2,3-c]pyridine |
| 98 | 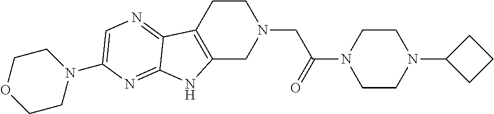 | 7-[2-(4-Cyclobutylpiperazin-1-yl)-2- oxoethyl]-3-morpholin-4-yl-6,7,8,9- tetrahydro-5H- pyrido[4′,3′:4,5]pyrrolo[2,3-b]pyrazine |
| 99 | 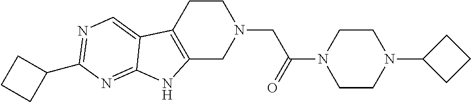 | 2-Cyclobutyl-7-[2-(4- cyclobutylpiperazin-1-yl)-2-oxoethyl]- 6,7,8,9-tetrahydro-5H- pyrido[4′,3′:4,5]pyrrolo[2,3- d]pyrimidine |
| 100 | 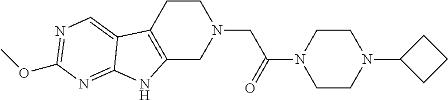 | 7-[2-(4-Cyclobutylpiperazin-1-yl)-2- oxoethyl]-2-methoxy-6,7,8,9- tetrahydro-5H-pyrido[4′,3′:4,5] pyrrolo[2,3-d]pyrimidine |
| 101 | 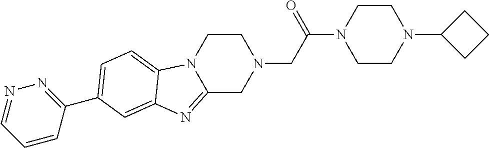 | 2-[2-(4-Cyclobutylpiperazin-1-yl)-2- oxoethyl]-8-pyridazin-3-yl-1,2,3,4- tetrahydropyrazino[1,2- a]benzimidazole |
| 102 | 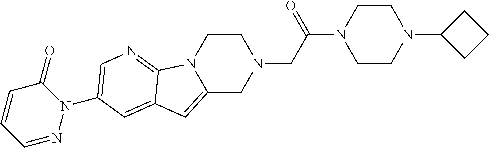 | 2-{7-[2-(4-Cyclobutylpiperazin-1-yl)- 2-oxoethyl]-6,7,8,9- tetrahydropyrido[3′,2′:4,5]pyrrolo[1,2- a]pyrazin-3-yl}pyridazin-3(2H)-one |
| 103 | 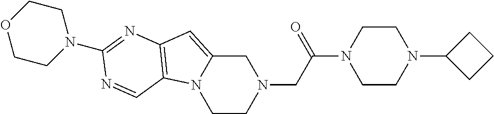 | 8-[2-(4-Cyclobutylpiperazin-1-yl)-2- oxoethyl]-2-morpholin-4-yl-6,7,8,9- tetrahydropyrazino[1′,2′:1,5]pyrrolo[3, 2-d]pyrimidine |
| 104 | 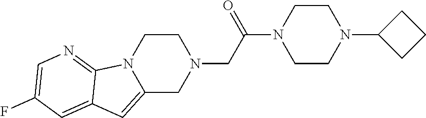 | 7-[2-(4-Cyclobutylpiperazin-1-yl)-2- oxoethyl]-3-fluoro-6,7,8,9- tetrahydropyrido[3′,2′:4,5]pyrrolo[1,2- a]pyrazine |
| 105 | 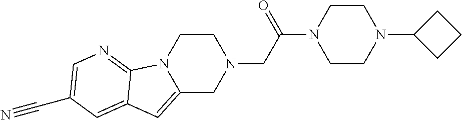 | 7-[2-(4-Cyclobutylpiperazin-1-yl)-2- oxoethyl]-6,7,8,9- tetrahydropyrido[3′,2′:4,5]pyrrolo[1,2- a]pyrazine-3-carbonitrile |
| 106 | 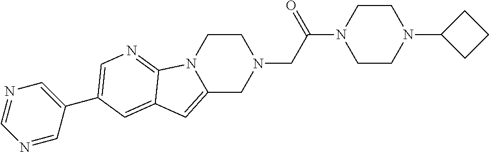 | 7-[2-(4-Cyclobutylpiperazin-1-yl)-2- oxoethyl]-3-pyrimidin-5-yl-6,7,8,9- tetrahydropyrido[3′,2′:4,5]pyrrolo[1,2- a]pyrazine |
| 107 | 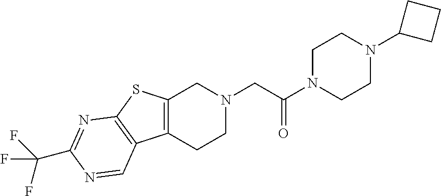 | 1-(4-Cyclobutyl-piperazin-1-yl)-2-(2- trifluoromethyl-5,8-dihydro-6H- pyrido[4′,3′:4,5]thieno[2,3- d]pyrimidin-7-yl)-ethanone |
| 108 | 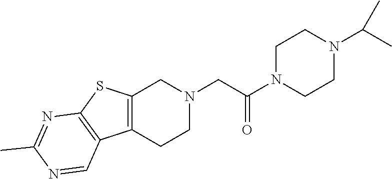 | 1-(4-Isopropyl-piperazin-1-yl)-2-(2- methyl-5,8-dihydro-6H- pyrido[4′,3′:4,5]thieno[2,3- d]pyrimidin-7-yl)-ethanone |
| 109 | 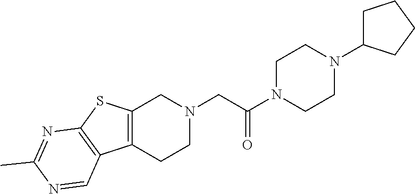 | 1-(4-Cyclopentyl-piperazin-1-yl)-2-(2- methyl-5,8-dihydro-6H- pyrido[4′,3′:4,5]thieno[2,3- d]pyrimidin-7-yl)-ethanone |
| 110 | 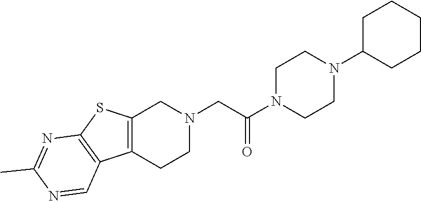 | 1-(4-Cyclohexyl-piperazin-1-yl)-2-(2- methyl-5,8-dihydro-6H- pyrido[4′,3:4,5]thieno[2,3-d]pyrimidin- 7-yl)-ethanone |
| 111 | 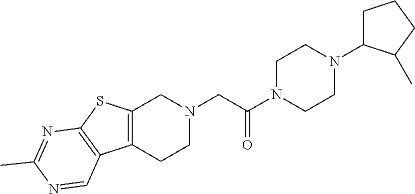 | 1-[4-(2-Methyl-cyclopentyl)-piperazin- 1-yl]-2-(2-methyl-5,8-dihydro-6H- pyrido[4′,3′:4,5]thieno[2,3- d]pyrimidin-7-yl)-ethanone |
| 112 | 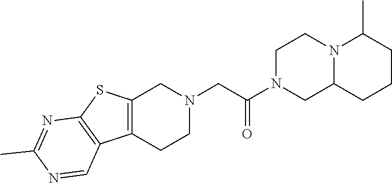 | 2-(2-Methyl-5,8-dihydro-6H- pyrido[4′,3′:4,5]thieno[2,3- d]pyrimidin-7-yl)-1-(6-methyl- octahydro-pyrido[1,2-a]pyrazin-2-yl)- ethanone |
| 113 | 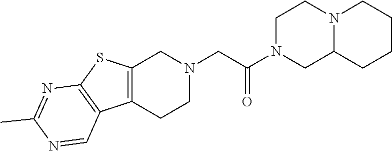 | 2-(2-Methyl-5,8-dihydro-6H- pyrido[4′,3′:4,5]thieno[2,3- d]pyrimidin-7-yl)-1-(octahydro- pyrido[1,2-a]pyrazin-2-yl)-ethanone |
| 114 | 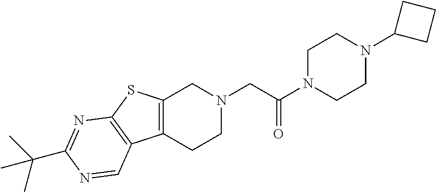 | 2-(2-tert-Butyl-5,8-dihydro-6H- pyrido[4′,3′:4,5]thieno[2,3- d]pyrimidin-7-yl)-1-(4-cyclobutyl- piperazin-1-yl)-ethanone |
| 115 | 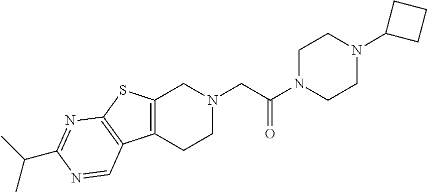 | 1-(4-Cyclobutyl-piperazin-1-yl)-2-(2- isopropyl-5,8-dihydro-6H- pyrido[4′,3′:4,5]thieno[2,3- d]pyrimidin-7-yl)-ethanone |
| 116 | 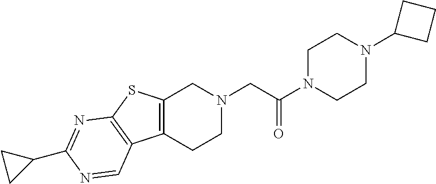 | 1-(4-Cyclobutyl-piperazin-1-yl)-2-(2- cyclopropyl-5,8-dihydro-6H- pyrido[4′,3′:4,5]thieno[2,3- d]pyrimidin-7-yl)-ethanone |
| 117 | 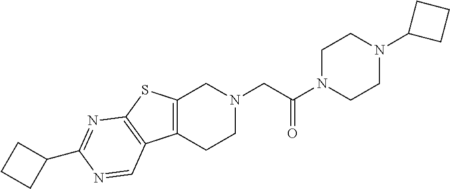 | 2-(2-Cyclobutyl-5,8-dihydro-6H- pyrido[4′,3′:4,5]thieno[2,3- d]pyrimidin-7-yl)-1-(4-cyclobutyl- piperazin-1-yl)-ethanone |
| 118 | 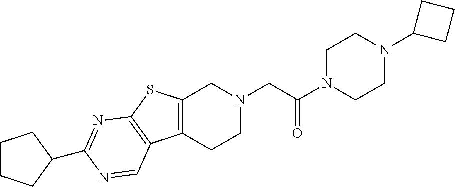 | 1-(4-Cyclobutyl-piperazin-1-yl)-2-(2- cyclopentyl-5,8-dihydro-6H- pyrido[4′,3′:4,5]thieno[2,3- d]pyrimidin-7-yl)-ethanone |
| 119 | 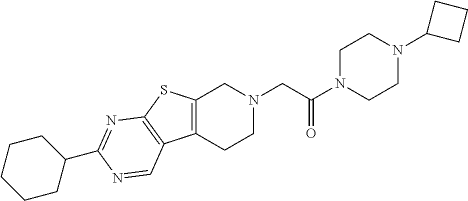 | 1-(4-Cyclobutyl-piperazin-1-yl)-2-(2- cyclohexyl-5,8-dihydro-6H- pyrido[4′,3′:4,5]thieno[2,3- d]pyrimidin-7-yl)-ethanone |
| 120 | 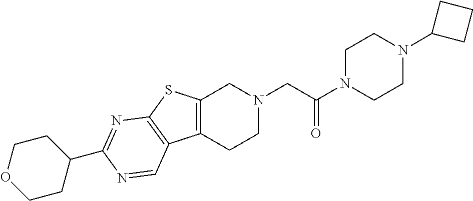 | 1-(4-Cyclobutyl-piperazin-1-yl)-2-[2- (tetrahydro-pyran-4-yl)-5,8-dihydro- 6H-pyrido[4′,3′:4,5]thieno[2,3- d]pyrimidin-7-yl]-ethanone |
| 121 | 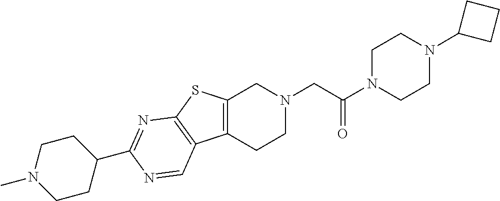 | 1-(4-Cyclobutyl-piperazin-1-yl)-2-[2- (1-methyl-piperidin-4-yl)-5,8-dihydro- 6H-pyrido[4′,3′:4,5]thieno[2,3- d]pyrimidin-7-yl]-ethanone |
| 122 | 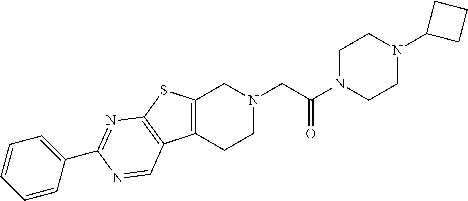 | 1-(4-Cyclobutyl-piperazin-1-yl)-2-(2- phenyl-5,8-dihydro-6H- pyrido[4′,3′:4,5]thieno[2,3- d]pyrimidin-7-yl)-ethanone |
| 123 | 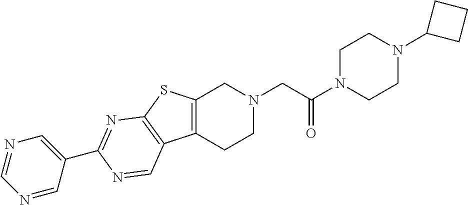 | 1-(4-Cyclobutyl-piperazin-1-yl)-2-(2- pyrimidin-5-yl-5,8-dihydro-6H- pyrido[4′,3′:4,5]thieno[2,3- d]pyrimidin-7-yl)-ethanone |
| 124 | 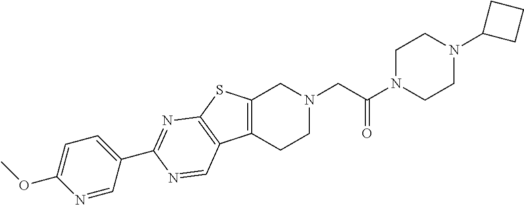 | 1-(4-Cyclobutyl-piperazin-1-yl)-2-[2- (6-methoxy-pyridin-3-yl)-5,8-dihydro- 6H-pyrido[4′,3′:4,5]thieno[2,3- d]pyrimidin-7-yl]-ethanone |
| 125 | 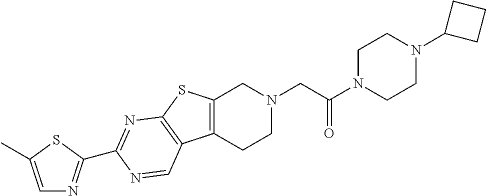 | 1-(4-Cyclobutyl-piperazin-1-yl)-2-[2- (5-methyl-thiazol-2-yl)-5,8-dihydro- 6H-pyrido[4′,3′:4,5]thieno[2,3- d]pyrimidin-7-yl]-ethanone |
| 126 | 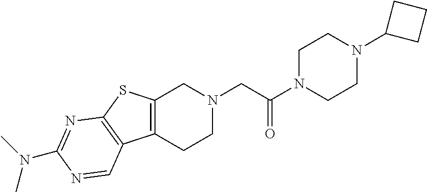 | 1-(4-Cyclobutyl-piperazin-1-yl)-2-(2- dimethylamino-5,8-dihydro-6H- pyrido[4′,3′:4,5]thieno[2,3- d]pyrimidin-7-yl)-ethanone |
| 127 | 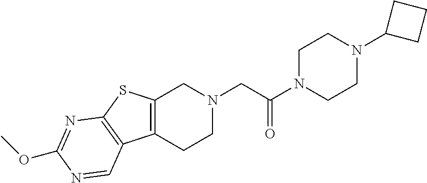 | 1-(4-Cyclobutyl-piperazin-1-yl)-2-(2- methoxy-5,8-dihydro-6H- pyrido[4′,3′:4,5]thieno[2,3- d]pyrimidin-7-yl)-ethanone |
| 128 | 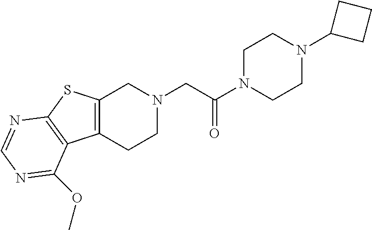 | 1-(4-Cyclobutyl-piperazin-1-yl)-2-(4- methoxy-5,8-dihydro-6H- pyrido[4′,3′:4,5]thieno[2,3- d]pyrimidin-7-yl)-ethanone |
| 129 | 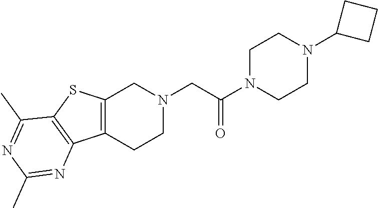 | 1-(4-Cyclobutyl-piperazin-1-yl)-2-(2,4- dimethyl-8,9-dihydro-6H- pyrido[4′,3′:4,5]thieno[3,2- d]pyrimidin-7-yl)-ethanone |
| 130 | 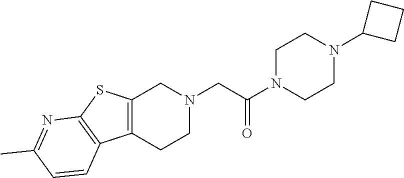 | 1-(4-Cyclobutyl-piperazin-1-yl)-2-(2- methyl-5,8-dihydro-6H-9-thia-1,7- diaza-fluoren-7-yl)-ethanone |
| 131 | 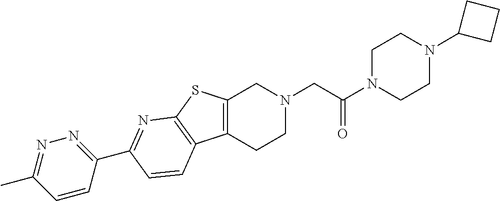 | 1-(4-Cyclobutyl-piperazin-1-yl)-2-[2- (6-methyl-pyridazin-3-yl)-5,8-dihydro- 6H-9-thia-1,7-diaza-fluoren-7-yl]- ethanone |
| 132 | 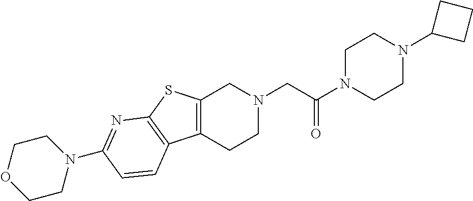 | 1-(4-Cyclobutyl-piperazin-1-yl)-2-(2- morpholin-4-yl-5,8-dihydro-6H-9-thia- 1,7-diaza-fluoren-7-yl)-ethanone |
| 133 | 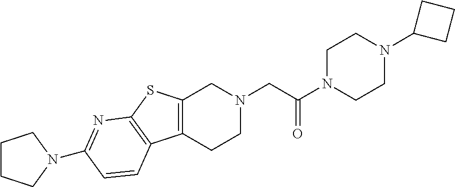 | 1-(4-Cyclobutyl-piperazin-1-yl)-2-(2- pyrrolidin-1-yl-5,8-dihydro-6H-9-thia- 1,7-diaza-fluoren-7-yl)-ethanone |
| 134 | 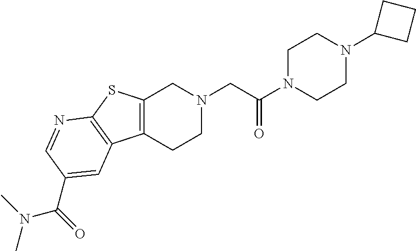 | 7-[2-(4-Cyclobutyl-piperazin-1-yl)-2- oxo-ethyl]-5,6,7,8-tetrahydro-9-thia- 1,7-diaza-fluorene-3-carboxylic acid dimethylamide |
| 135 | 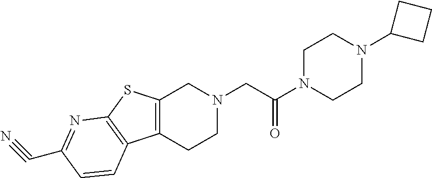 | 7-[2-(4-Cyclobutyl-piperazin-1-yl)-2- oxo-ethyl]-5,6,7,8-tetrahydro-9-thia- 1,7-diaza-fluorene-2-carbonitrile |
| 136 | 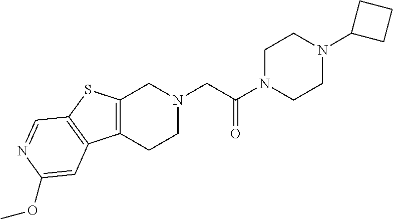 | 1-(4-Cyclobutyl-piperazin-1-yl)-2-(6- methoxy-3,4-dihydro-1H-thieno[2,3- c;5,4-c′]dipyridin-2-yl)-ethanone |
| 137 | 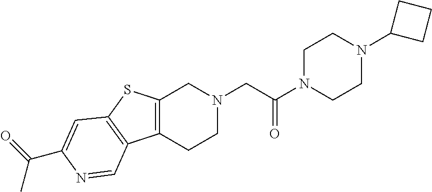 | 2-(7-Acetyl-3,4-dihydro-1H-9-thia-2,6- diaza-fluoren-2-yl)-1-(4-cyclobutyl- piperazin-1-yl)-ethanone |
| 138 | 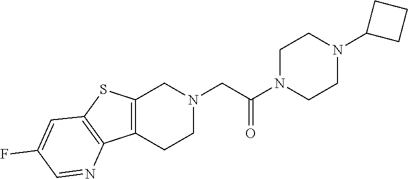 | 1-(4-Cyclobutyl-piperazin-1-yl)-2-(7- fluoro-3,4-dihydro-1H-9-thia-2,5- diaza-fluoren-2-yl)-ethanone |
| 139 | 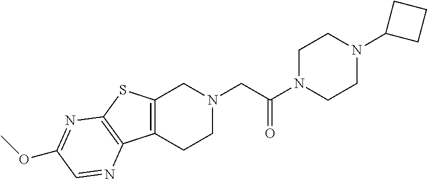 | 1-(4-Cyclobutyl-piperazin-1-yl)-2-(2- methoxy-5,8-dihydro-6H-9-thia-1,4,7- triaza-fluoren-7-yl)-ethanone |
| 140 | 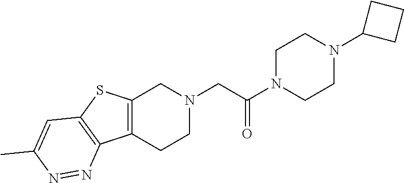 | 1-(4-Cyclobutyl-piperazin-1-yl)-2-(7- methyl-3,4-dihydro-1H-9-thia-2,5,6- triaza-fluoren-2-yl)-ethanone |
| 141 | 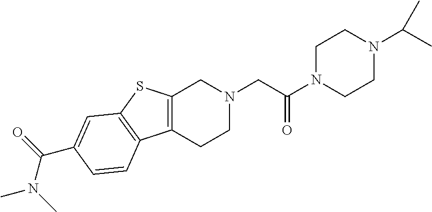 | 2-[2-(4-Isopropyl-piperazin-1-yl)-2- oxo-ethyl]-1,2,3,4-tetrahydro- benzo[4,5]thieno[2,3-c]pyridine-7- carboxylic acid dimethylamide |
| 142 | 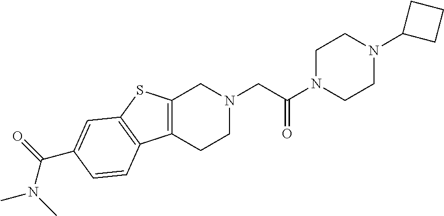 | 2-[2-(4-Cyclobutyl-piperazin-1-yl)-2- oxo-ethyl]-1,2,3,4-tetrahydro- benzo[4,5]thieno[2,3-c]pyridine-7- carboxylic acid dimethylamide |
| 143 | 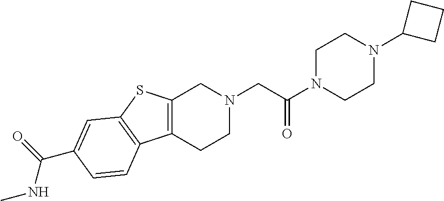 | 2-[2-(4-Cyclobutyl-piperazin-1-yl)-2- oxo-ethyl]-1,2,3,4-tetrahydro- benzo[4,5]thieno[2,3-c]pyridine-7- carboxylic acid methylamide |
| 144 | 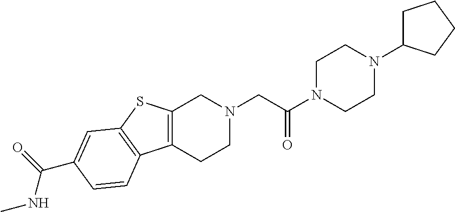 | 2-[2-(4-Cyclopentyl-piperazin-1-yl)-2- oxo-ethyl]-1,2,3,4-tetrahydro- benzo[4,5]thieno[2,3-c]pyridine-7- carboxylic acid methylamide |
| 145 | 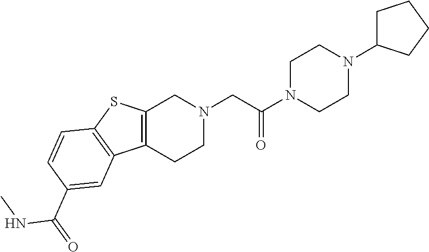 | 2-[2-(4-Cyclopentyl-piperazin-1-yl)-2- oxo-ethyl]-1,2,3,4-tetrahydro- benzo[4,5]thieno[2,3-c]pyridine-6- carboxylic acid methylamide |
| 146 | 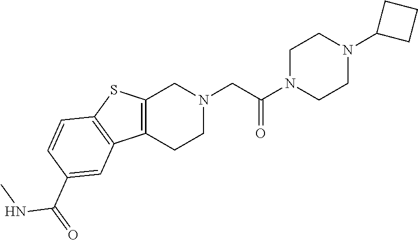 | 2-[2-(4-Cyclobutyl-piperazin-1-yl)-2- oxo-ethyl]-1,2,3,4-tetrahydro- benzo[4,5]thieno[2,3-c]pyridine-6- carboxylic acid methylamide |
| 147 | 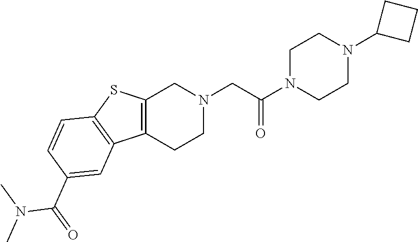 | 2-[2-(4-Cyclobutyl-piperazin-1-yl)-2- oxo-ethyl]-1,2,3,4-tetrahydro- benzo[4,5]thieno[2,3-c]pyridine-6- carboxylic acid dimethylamide |
| 148 | 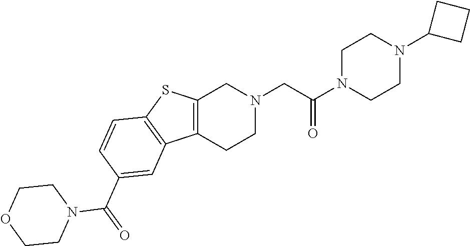 | 1-(4-Cyclobutyl-piperazin-1-yl)-2-[6- (morpholine-4-carbonyl)-3,4-dihydro- 1H-benzo[4,5]thieno[2,3-c]pyridin-2- yl]-ethanone |
| 149 | 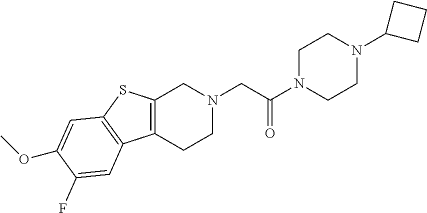 | 1-(4-Cyclobutyl-piperazin-1-yl)-2-(6- fluoro-7-methoxy-3,4-dihydro-1H- benzo[4,5]thieno[2,3-c]pyridin-2-yl)- ethanone |
| 150 | 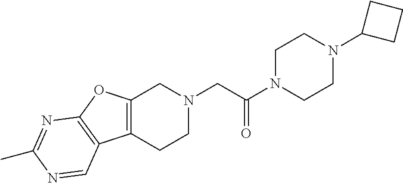 | 1-(4-Cyclobutyl-piperazin-1-yl)-2-(2- methyl-5,8-dihydro-6H- pyrido[4′,3′:4,5]furo[2,3-d]pyrimidin- 7-yl)-ethanone |
| 151 | 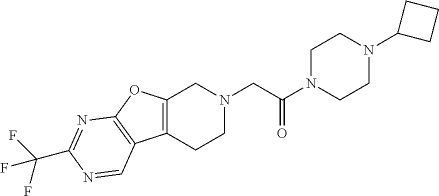 | 1-(4-Cyclobutyl-piperazin-1-yl)-2-(2- trifluoromethyl-5,8-dihydro-6H- pyrido[4′,3′:4,5]furo[2,3-d]pyrimidin- 7-yl)-ethanone |
| 152 | 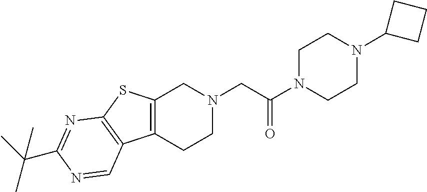 | 2-(2-tert-Butyl-5,8-dihydro-6H- pyrido[4′,3′:4,5]furo[2,3-d]pyrimidin- 7-yl)-1-(4-cyclobutyl-piperazin-1-yl)- ethanone |
| 153 | 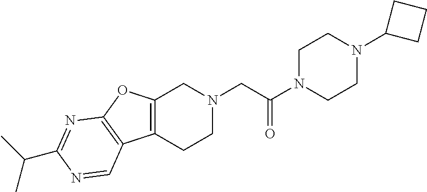 | 1-(4-Cyclobutyl-piperazin-1-yl)-2-(2- isopropyl-5,8-dihydro-6H- pyrido[4′,3′:4,5]furo[2,3-d]pyrimidin- 7-yl)-ethanone |
| 154 | 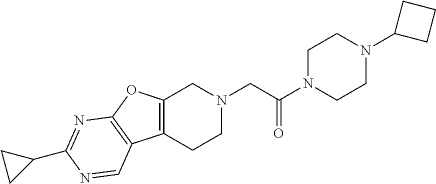 | 1-(4-Cyclobutyl-piperazin-1-yl)-2-(2- cyclopropyl-5,8-dihydro-6H- pyrido[4′,3′:4,5]furo[2,3-d]pyrimidin- 7-yl)-ethanone |
| 155 | 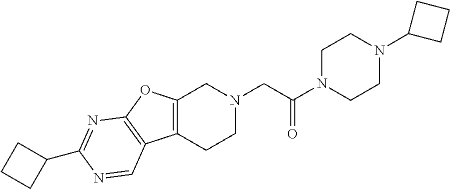 | 2-(2-Cyclobutyl-5,8-dihydro-6H- pyrido[4′,3′:4,5]furo[2,3-d]pyrimidin- 7-yl)-1-(4-cyclobutyl-piperazin-1-yl)- ethanone |
| 156 | 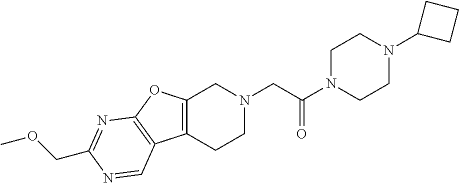 | 1-(4-Cyclobutyl-piperazin-1-yl)-2-(2- methoxymethyl-5,8-dihydro-6H- pyrido[4′,3′:4,5]furo[2,3-d]pyrimidin- 7-yl)-ethanone |
| 157 | 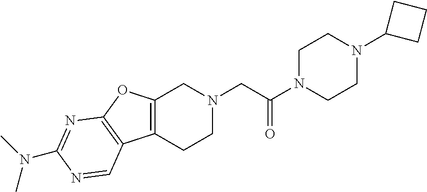 | 1-(4-Cyclobutyl-piperazin-1-yl)-2-(2- dimethylamino-5,8-dihydro-6H- pyrido[4′,3′:4,5]furo[2,3-d]pyrimidin- 7-yl)-ethanone |
| 158 | 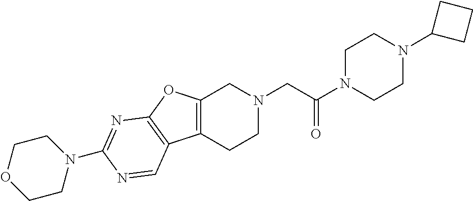 | 1-(4-Cyclobutyl-piperazin-1-yl)-2-(2- morpholin-4-yl-5,8-dihydro-6H- pyrido[4′,3′:4,5]furo[2,3-d]pyrimidin- 7-yl)-ethanone |
| 159 | 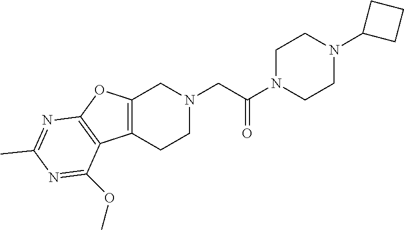 | 1-(4-Cyclobutyl-piperazin-1-yl)-2-(4- methoxy-2-methyl-5,8-dihydro-6H- pyrido[4′,3′:4,5]furo[2,3-d]pyrimidin- 7-yl)-ethanone |
| 160 | 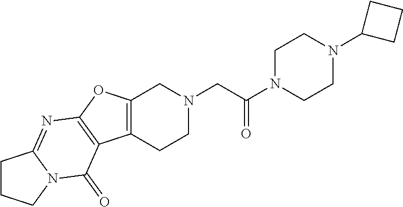 | 7-[2-(4-Cyclobutyl-piperazin-1-yl)-2- oxo-ethyl]-2,3,5,6,7,8-hexahydro-1H- 9-oxa-3a,7,10-triaza- cyclopenta[b]fluoren-4-one |
| 161 | 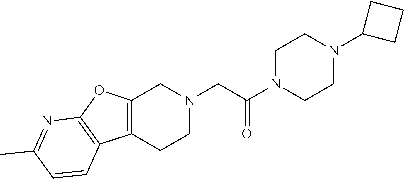 | 1-(4-Cyclobutyl-piperazin-1-yl)-2-(2- methyl-5,8-dihydro-6H-9-oxa-1,7- diaza-fluoren-7-yl)-ethanone |
| 162 | 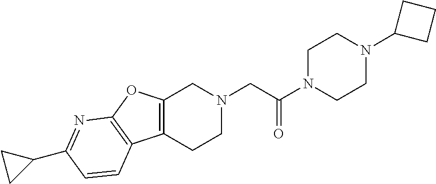 | 1-(4-Cyclobutyl-piperazin-1-yl)-2-(2- cyclopropyl-5,8-dihydro-6H-9-oxa-1,7- diaza-fluoren-7-yl)-ethanone |
| 163 | 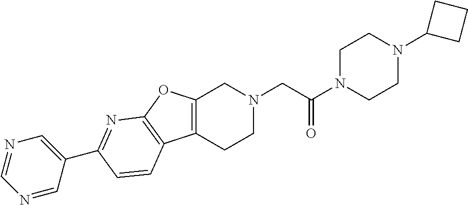 | 1-(4-Cyclobutyl-piperazin-1-yl)-2-(2- pyrimidin-5-yl-5,8-dihydro-6H-9-oxa- 1,7-diaza-fluoren-7-yl)-ethanone |
| 164 | 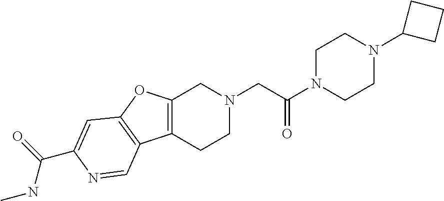 | 7-[2-(4-Cyclobutyl-piperazin-1-yl)-2- oxo-ethyl]-6,7,8,9-tetrahydro-furo[2,3- c;4,5-c′]dipyridine-3-carboxylic acid methylamide |
| 165 | 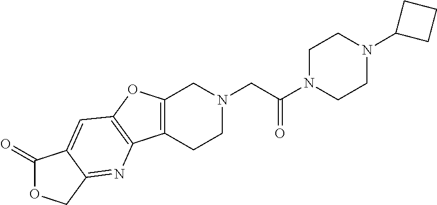 | 7-[2-(4-Cyclobutyl-piperazin-1-yl)-2- oxo-ethyl]-5,6,7,8-tetrahydro-3H-2,9- dioxa-4,7-diaza-cyclopenta[b]fluoren- 1-one |
| 166 | 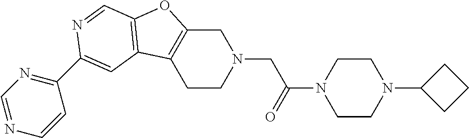 | 1-(4-Cyclobutyl-piperazin-1-yl)-2-(6- pyrimidin-4-yl-3,4-dihydro-1H- furo[2,3-c;5,4-c′]dipyridin-2-yl)- ethanone |
| 167 | 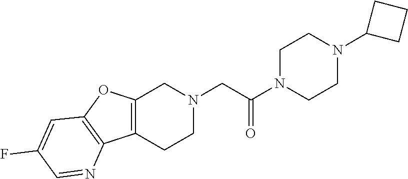 | 1-(4-Cyclobutyl-piperazin-1-yl)-2-(7- fluoro-3,4-dihydro-1H-9-oxa-2,5- diaza-fluoren-2-yl)-ethanone |
| 168 | 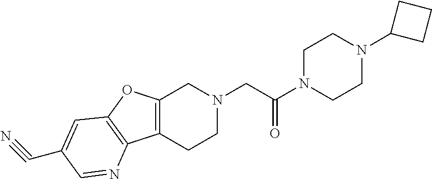 | 2-[2-(4-Cyclobutyl-piperazin-1-yl)-2- oxo-ethyl]-1,2,3,4-tetrahydro-9-oxa- 2,5-diaza-fluorene-7-carbonitrile |
| 169 | 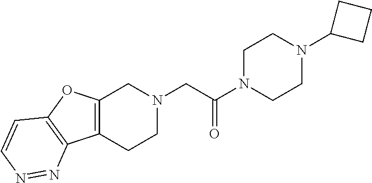 | 1-(4-Cyclobutyl-piperazin-1-yl)-2-(3,4- dihydro-1H-9-oxa-2,5,6-triaza-fluoren- 2-yl)-ethanone |
| 170 | 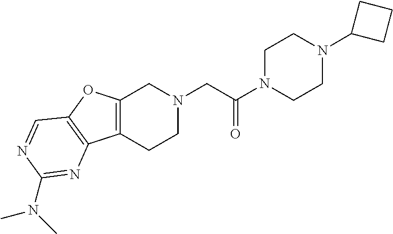 | 1-(4-Cyclobutyl-piperazin-1-yl)-2-(3- dimethylamino-5,8-dihydro-6H-9-oxa- 2,4,7-triaza-fluoren-7-yl)-ethanone |
| 171 | 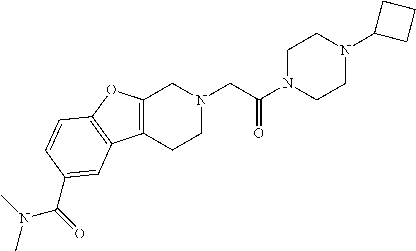 | 2-[2-(4-Cyclobutyl-piperazin-1-yl)-2- oxo-ethyl]-1,2,3,4-tetrahydro- benzo[4,5]furo[2,3-c]pyridine-6- carboxylic acid dimethylamide |
| 172 | 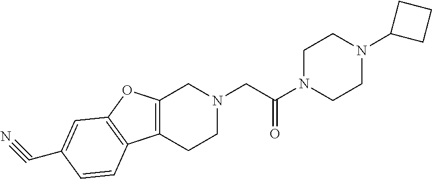 | 2-[2-(4-Cyclobutyl-piperazin-1-yl)-2- oxo-ethyl]-1,2,3,4-tetrahydro- benzo[4,5]furo[2,3-c]pyridine-7- carbonitrile |
| 173 | 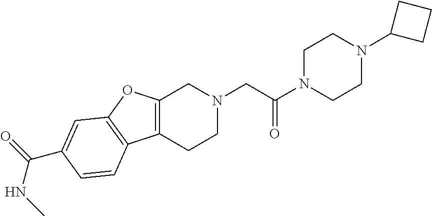 | 2-[2-(4-Cyclobutyl-piperazin-1-yl)-2- oxo-ethyl]-1,2,3,4-tetrahydro- benzo[4,5]furo[2,3-c]pyridine-7- carboxylic acid methylamide |
| 174 | 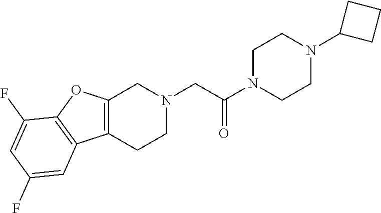 | 1-(4-Cyclobutyl-piperazin-1-yl)-2-(6,8- difluoro-3,4-dihydro-1H- benzo[4,5]furo[2,3-c]pyridin-2-yl)- ethanone |

### Example 4

### Preparation of Chimeric Human H3 Receptor

Chimeric H3 receptor cDNA from human H3 receptor is generated from three cDNA fragments: (1) a human H3 receptor cDNA 5′ fragment; (2) a human H3 receptor cDNA 3′ fragment; and (3) a rat Gαᵢ₂ cDNA fragment, each containing appropriate, overlapping linker sequences, as described in Example 1 of U.S. patent application Ser. No. 11/355,711, which published as US 2006/0188960, and is hereby incorporated by reference for its teaching of the preparation of a chimeric human H3 receptor-rat Gαᵢ₂ baculoviral expression construct that has the sequence provided in SEQ ID NO:7 of US 2006/0188960, and encodes a polypeptide that has the sequence provided in SEQ ID NO:8 of US 2006/0188960.

### Example 5

### Chimeric Human H3 Receptor Baculovirus Preparation and Infection

The chimeric human H3 receptor-rat Gaαᵢ₂ baculoviral expression vector is co-transfected along with BACULOGOLD DNA (BD PHARMINGEN, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Kans.) supplemented Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are chosen for a second round of amplification, using 1 ml of passage 1 stock to infect 1×10⁸ cells in 100 ml of insect medium divided into two T175 flasks. Forty-eight hours post infection, passage 2 medium from each 100 ml prep is harvested and plaque assayed to determine virus titer. The cell pellets from the second round of amplification are assayed by affinity binding as described below to verify recombinant receptor expression. A third round of amplification is then initiated using a multiplicity of infection of 0.1 to infect a liter of Sf9 cells. Forty hours post-infection, the supernatant medium is harvested to yield passage 3 baculoviral stock.

The remaining cell pellet is assayed for affinity binding using the protocol of DeMartino et al. (1994) J. Biol. Chem. 269(20):14446-50 (which is incorporated herein by reference for its teaching of binding assays at page 14447), adapted as follows. Radioligand ranges from 0.40-40 nM [³H]-N-(a)methylhistamine (Perkin Elmer, Boston, Mass.) and assay buffer contains 50 mM Tris, 1 mM CaCl₂, 5 mM MgCl₂, 0.1% BSA, 0.1 mM bacitracin, and 100 KIU/ml aprotinin, pH 7.4. Filtration is carried out using GF/C WHATMAN filters (presoaked in 1.0% polyethyeneimine for 2 hr prior to use). Filters are washed three times with 5 ml cold assay buffer without BSA, bacitracin, or aprotinin and air dried for 12-16 hr. Radioactivity retained on filters is measured on a beta scintillation counter.

Titer of the passage 3 baculoviral stock is determined by plaque assay and a multiplicity of infection, incubation time course, binding assay experiment is carried out to determine conditions for optimal receptor expression. A multiplicity of infection of 0.5 and a 72-hr incubation period are preferred infection parameters for chimeric human H3 receptor-rat Gaαᵢ₂ expression in up to 1-liter Sf9 cell infection cultures.

Log-phase Sf9 cells (INVITROGEN), are infected with one or more stocks of recombinant baculovirus followed by culturing in insect medium at 27° C. Infections are carried out with virus directing the expression of human H3 receptor-rat Gaα₂ in combination with three G-protein subunit-expression virus stocks: 1) rat Gaα₂ G-protein-encoding virus stock (BIOSIGNAL #V5J008), 2) bovine β1 G-protein-encoding virus stock (BIOSIGNAL #V5H012), and 3) human γ2 G-protein-encoding virus stock (BIOSIGNAL #V6B003), which may be obtained from BIOSIGNAL Inc., Montreal.

The infections are conveniently carried out at a multiplicity of infection of 0.5:1.0:0.5:0.5. At 72 hr post-infection, an aliquot of cell suspension is analyzed for viability by trypan blue dye exclusion. If no blue is detected by visual inspection, the Sf9 cells are harvested via centrifugation (3000 rpm/10 min/4° C.).

### Example 6

### Chimeric Human H3 Receptor Cell Membrane Preparations

Sf9 cell pellets obtained as described in Example 5 are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON PT10-35 homogenizer (KINEMATICA AG, Lucerne, Switzerland; setting 5 for 30 seconds). The homogenate is centrifuged (536×g/10 min at 4° C.) to pellet the nuclei and unbroken cells. The supernatant containing the membranes is decanted to a clean centrifuge tube, centrifuged (48,000×g/30 min, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until used for radioligand binding or functional response assays. The protein concentration of the resulting membrane preparation (hereinafter termed “P2 membranes”) is conveniently measured using a Bradford protein assay (BIO-RAD LABORATORIES, Hercules, Calif.). By this measure, a 1-liter culture of cells typically yields 100-150 mg of total membrane protein.

### Example 7

### Chimeric Human H3 Receptor GTP Binding Assays

This Example illustrates a representative assay for evaluating agonist-stimulated GTP-gamma³⁵S binding (“GTP binding”) activity. Such GTP binding activity can be used to identify H3 antagonists and to differentiate neutral antagonist compounds from those that possess inverse agonist activity. This agonist-stimulated GTP binding activity can also be used to detect partial agonism mediated by antagonist compounds. A compound analyzed in this assay is referred to herein as a “test compound.”

Four independent baculoviral stocks (one directing the expression of the chimeric human H3 receptor and three directing the expression of each of the three subunits of a heterotrimeric G-protein) are used to infect a culture of Sf9 cells as described above. P2 membranes are prepared as described above, and agonist-stimulated GTP binding on the P2 membranes is assessed using histamine (Sigma Chemical Co., St. Louis, Mo.) as agonist in order to ascertain that the receptor/G-protein-alpha-beta-gamma combination(s) yield a functional response as measured by GTP binding. P2 membranes are resuspended by Dounce homogenization (tight pestle) in GTP binding assay buffer (50 mM Tris pH 7.4, 120 mM NaCl, 5 mM MgCl₂, 2 mM EGTA, 1 mg/ml BSA, 0.2 mg/ml bacitracin, 0.02 mg/ml aprotinin, 0.01 mg/ml saponin, 10 μM GDP) and added to assay tubes at a concentration of 35 μg protein/reaction tube. After adding increasing doses of histamine at concentrations ranging from 10⁻¹² M to 10⁻⁵ M, reactions are initiated by the addition of 125 pM GTP-gamma³⁵S (PERKIN ELMER; Boston, Mass.) with a final assay volume of 0.20 ml. In competition experiments, non-radiolabeled test compounds are added to separate reactions at concentrations ranging from 10⁻¹⁰ M to 10⁻⁶M along with 1 μM histamine to yield a final volume of 0.20 ml.

Neutral antagonists are antagonists that are substantially free of inherent agonist activity, and include those test compounds that reduce the histamine-stimulated GTP binding activity towards, but not below, baseline levels. In contrast, in the absence of added histamine, inverse agonists reduce the GTP binding activity of the receptor-containing membranes below baseline. The elevation of GTP binding activity above baseline by a compound in the absence of added histamine in this assay demonstrates agonist activity.

After a 60-min incubation at room temperature, reactions are terminated by vacuum filtration over WHATMAN GF/C filters (pre-soaked in wash buffer, 0.1% BSA) followed by washing with ice-cold wash buffer (50 mM Tris pH 7.4, 120 mM NaCl). The amount of receptor-bound (and thereby membrane-bound) GTE-gamma³⁵S is determined by measuring the filter-bound radioactivity, preferably by liquid scintillation spectrometry of the washed filters. Non-specific binding is determined in parallel assays including 10 μM unlabeled GTP-gammaS and typically represents less than 5 percent of total binding. Data is expressed as percent above basal (baseline). The results of GTP binding experiments are analyzed using SIGMAPLOT software (SPSS Inc., Chicago, Ill.). IC₅₀ values are calculated by non-linear regression analysis of dose-response curves using Kaleidograph (Synergy Software, Reading, Pa.).

Alternatively the data is analyzed as follows. First, the average bound radioactivity from negative control wells (no agonist) is subtracted from the bound radioactivity detected for each of the other experimental wells. Second, average bound radioactivity is calculated for the positive control wells (agonist wells). Then, percent inhibition for each compound tested is calculated using the equation:

$$Percent Inhibition = 100 - 100 [ \frac{Bound radioactivity in Test Wells}{Bound radioactivity in Agonist} ] \times Wells$$

The % inhibition data is plotted as a function of test compound concentration and test compound IC₅₀ is determined using a linear regression in which x is ln (concentration of test compound) and y is ln (percent inhibition/(100−percent inhibition). Data with a percent inhibition that is greater than 90% or less than 15% are rejected and are not used in the regression. The IC₅₀ is e^{(-intercept/slope)}.

Calculated IC₅₀ values are converted to Kᵢ values by the Cheng-Prusoff correction (Cheng and Prusoff (1973) Biochem. Pharmacol. 22(23):3099-3108). Accordingly, the following equation: Kᵢ=IC₅₀/(1+[L]/EC₅₀) is used, where [L] is the histamine concentration in the GTP binding assay, and EC₅₀ is the concentration of histamine producing a 50% response, as determined by a dose-response analysis using concentrations of histamine ranging from 10⁻¹⁰ M to 10⁻⁶M.

To assess agonist or inverse agonist activity of a test compound, this assay is performed in the absence of added histamine, and EC₅₀ values are determined by analogous calculations, where the EC₅₀ is the concentration of test compound producing a 50% response.

### Example 8

### Chimeric Human H3 Receptor Screening: GTP Binding Assays

This Example illustrates a representative screening assay for evaluating inhibition of histamine-stimulated GTP-gamma³⁵S binding. Such GTP binding activity can be used to identify H3 antagonists and inverse agonists. A compound analyzed in this assay is referred to herein as a “test compound,” and the initial identification of antagonists and inverse agonists is performed using a test compound concentration of 4 μM.

Four independent baculoviral stocks (one directing the expression of the chimeric human H3 receptor and three directing the expression of each of the three subunits of a heterotrimeric G-protein) are used to infect a culture of Sf9 cells as described above. P2 membranes are prepared as described above, and are resuspended by Dounce homogenization (tight pestle) in GTP binding assay buffer (50 mM Tris pH 7.4, 120 mM NaCl, 5 mM MgCl₂, 2 mM EGTA, 1 mg/ml BSA, 0.2 mg/ml bacitracin, 0.02 mg/ml aprotinin, 0.01 mg/ml saponin, 10 μM GDP) and added to assay tubes at a concentration of 35 μg protein/reaction tube. Non-radiolabeled test compounds are added to separate reactions at a concentration of 4 μM along with 1 μM histamine (agonist). Reactions are initiated by the addition of 125 pM GTP-gamma³⁵S with a final assay volume of 0.20 ml.

After a 60-min incubation at room temperature, reactions are terminated by vacuum filtration over GF/C filters (pre-soaked in 50 mM Tris pH 7.4, 120 mM NaCl plus 0.1% BSA) followed by washing with ice-cold buffer (50 mM Tris pH 7.4, 120 mM NaCl). The amount of receptor-bound (and thereby membrane-bound) GTP-gamma³⁵S is determined by measuring the bound radioactivity, preferably by liquid scintillation spectrometry of the washed filters. Non-specific binding is determined using 10 uM GTP-gammaS and typically represents less than 5 percent of total binding. After subtraction of non-specific binding, data is expressed as percent inhibition of 1 μM histamine signal.

Neutral antagonists are those test compounds that reduce the histamine-stimulated GTP binding activity towards, but not below, baseline levels. In contrast, in the absence of added histamine, inverse agonists reduce the GTP binding activity of the receptor-containing membranes below baseline. Any test compound that elevates GTP binding activity above baseline in the absence of added histamine in this assay is defined as having agonist activity.

1. A compound of the Formula: 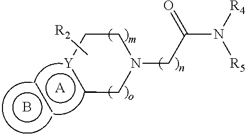
or a pharmaceutically acceptable salt or hydrate thereof, wherein:
Y is C or N; 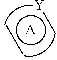
represents a 5- or 6-membered heteroaryl that is fused to the ring represented by 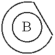
and is also fused to the ring represented by 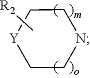 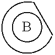
represents phenyl or a 5- or 6-membered heteroaryl that is fused to the ring represented by 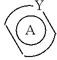
each of which phenyl or heteroaryl is substituted with from 0 to 4 substituents independently chosen from:
(i) hydrogen, amino, halogen, cyano, hydroxy, nitro and oxo; and
(ii) C₁-C₆alkyl, C₂-C₆alkenyl, C₂-C₆alkynyl, (C₃-C₈cycloalkyl)C₀-C₂alkyl, C₁-C₆alkoxy, C₁-C₆haloalkyl, C₂-C₆alkyl ether, C₁-C₆alkylsulfonyl, mono- or di-(C₁-C₆alkyl)aminoC₀-C₄alkyl, phenylC₀-C₂alkyl or (5- to 7-membered heterocycle)C₀-C₂alkyl; each of which is unsubstituted or substituted with oxo, C₁-C₆alkyl or C₁-C₆alkoxy;
n is 0, 1, 2 or 3;
m is 0, 1 or 2;
o is 1 or 2;
R₂ represents from 0 to 4 substituents independently chosen from C₃-C₆alkyl, C₂-C₆alkenyl, C₂-C₆alkynyl, (C₃-C₈cycloalkyl)C₀-C₂alkyl, C₁-C₆haloalkyl, and groups that are taken together to form a C₁-C₃alkylene bridge;
R₄ and R₅ are:
(i) independently chosen from C₁-C₆alkyl, C₂-C₆alkenyl, C₂-C₆alkynyl, C₁-C₆haloalkyl and C₂-C₆alkyl ether; each of which is substituted with from 0 to 4 substituents independently chosen from amino, cyano, oxo, mono- or di-(C₁-C₆alkyl)amino, mono- or di-(C₁-C₆alkyl)aminocarbonyl, and 5- to 7-membered heterocycloalkyl; such that at least one of R₄ and R₅ is substituted with a nitrogen-containing heterocycle or an amine; or
(ii) taken together to form a 4- to 10-membered heterocycloalkyl that is substituted with from 0 to 4 substituents independently chosen from C₁-C₆alkyl, C₂-C₆alkenyl, C₂-C₆alkynyl, C₁-C₆haloalkyl, mono- or di-(C₁-C₆alkyl)aminoC₀-C₂alkyl, (C₃-C₈cycloalkyl)C₀-C₂alkyl, phenylC₀-C₂alkyl (4- to 8-membered heterocycloalkyl)C₀-C₂alkyl and groups that are taken together to form a C₁-C₃alkylene bridge; each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl.

2. A compound or salt or hydrate thereof according to claim 1, wherein the compound satisfies the formula: 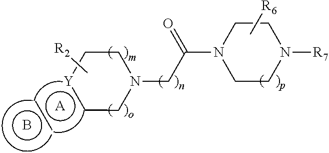
wherein:
p is 1, 2 or 3;
R₆ represents from 0 to 4 substituents independently chosen from C₁-C₆alkyl, C₁-C₆haloalkyl and groups that are taken together to form a C₁-C₃alkylene bridge; and
R₇ is C₁-C₆alkyl, C₂-C₆alkenyl, C₂-C₆alkynyl, (C₃-C₈cycloalkyl)C₀-C₂alkyl, phenylC₀-C₂alkyl or (4- to 8-membered heterocycloalkyl)C₀-C₂alkyl, each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl.

3-4. (canceled)

5. A compound of the Formula: 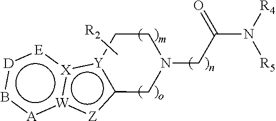
or a pharmaceutically acceptable salt or hydrate thereof, wherein:
A, B, D and E are independently N or CR₁; such that 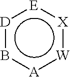
is aromatic;
W, X and Y are independently C or N;
Z is CR₉, N, NR₃, S or O;
n is 0, 1, 2 or 3;
m is 0, 1 or 2;
o is 1 or 2;
Each R₁ is independently:
(i) hydrogen, amino, halogen, cyano, hydroxy, nitro or oxo; or
(ii) C₁-C₆alkyl, C₂-C₆alkenyl, C₂-C₆alkynyl, (C₃-C₈cycloalkyl)C₀-C₂alkyl, C₁-C₆alkoxy, C₁-C₆haloalkyl, C₂-C₆alkyl ether, C₁-C₆alkylsulfonyl, mono- or di-(C₁-C₆alkyl)aminoC₀-C₄alkyl, phenylC₀-C₂alkyl or (5- to 7-membered heterocycle)C₀-C₂alkyl; each of which is unsubstituted or substituted with oxo, C₁-C₆alkyl or C₁-C₆alkoxy;
R₂ represents from 0 to 4 substituents independently chosen from C₂-C₆alkyl, C₂-C₆alkenyl, C₂-C₆alkynyl, (C₃-C₈cycloalkyl)C₀-C₂alkyl, C₁-C₆haloalkyl and groups that are taken together to form a C₁-C₃alkylene bridge;
R₃ is hydrogen, C₁-C₆alkyl, C₂-C₆alkenyl, C₂-C₆alkynyl, C₁-C₆haloalkyl, C₂-C₆alkyl ether, C₂-C₆-aminoalkyl, or mono- or di-(C₁-C₆alkyl)aminoC₂-C₆alkyl;
R₄ and R₅ are:
(i) independently chosen from C₁-C₆alkyl, C₂-C₆alkenyl, C₂-C₆alkynyl, C₁-C₆haloalkyl and C₂-C₆alkyl ether; each of which is substituted with from 0 to 4 substituents independently chosen from amino, cyano, oxo, mono- or di-(C₁-C₆alkyl)amino, mono- or di-(C₁-C₆alkyl)aminocarbonyl, and 5- to 7-membered heterocycloalkyl; such that at least one of R₄ and R₅ is substituted with a nitrogen-containing heterocycle or an amine; or
(ii) taken together to form a 4- to 10-membered heterocycloalkyl that is substituted with from 0 to 4 substituents independently chosen from C₁-C₆alkyl, C₂-C₆alkenyl, C₂-C₆alkynyl, C₁-C₆haloalkyl, mono- or di-(C₁-C₆alkyl)aminoC₀-C₂alkyl, (C₃-C₈cycloalkyl)C₀-C₂alkyl, phenylC₀-C₂alkyl, (4- to 7-membered heterocycloalkyl)C₀-C₂alkyl and groups that are taken together to form a C₁-C₃alkylene bridge; each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl; and
R₉ is hydrogen, amino, C₁-C₆alkyl, C₂-C₆alkenyl, C₂-C₆alkynyl, C₁-C₆haloalkyl, C₂-C₆alkyl ether, C₁-C₆aminoalkyl, or mono- or di-(C₁-C₆alkyl)aminoC₀-C₆alkyl.

6. A compound or salt or hydrate thereof according to claim 5, wherein the compound satisfies the formula: 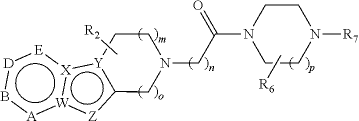
wherein:
p is 1, 2 or 3;
R₆ represents from 0 to 4 substituents independently chosen from C₁-C₆alkyl, C₁-C₆haloalkyl and groups that are taken together to form a C₁-C₃alkylene bridge; and
R₇ is C₁-C₆alkyl, C₂-C₆alkenyl, C₂-C₆alkynyl, (C₃-C₈cycloalkyl)C₀-C₂alkyl, phenylC₀-C₂alkyl or (4- to 8-membered heterocycloalkyl)C₀-C₂alkyl, each of which is substituted with from 0 to 4 substituents independently chosen from oxo, nitro, halogen, amino, cyano, hydroxy, aminocarbonyl, C₁-C₆alkyl, C₂-C₆alkenyl, C₁-C₆haloalkyl, C₁-C₆alkoxy, C₁-C₆haloalkoxy, C₁-C₆alkylthio, C₂-C₆ alkyl ether, C₁-C₆alkanoyl, C₃-C₆alkanone, C₁-C₆alkoxycarbonyl, mono- or di-(C₁-C₆alkyl)amino, mono- or di-(C₁-C₆alkyl)aminocarbonyl, C₃-C₇cycloalkyl and 4- to 7-membered heterocycloalkyl.

7.-9. (canceled)

10. A compound or salt or hydrate thereof according to claim 6, wherein:
X, W and Y are carbon; and
Z is nitrogen or NR₃.

11.-12. (canceled)

13. A compound or salt or hydrate thereof according to claim 10, wherein:
W is carbon; and
exactly one of A, B, D and E is nitrogen.

14.-16. (canceled)

17. A compound or salt or hydrate thereof according to claim 10, wherein: 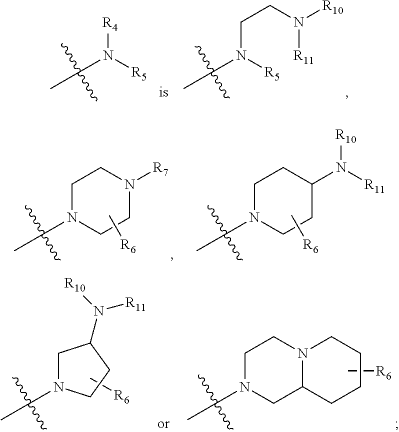
R₅ is hydrogen or C₁-C₆alkyl;
R₆ represents from 0 to 2 substituents independently chosen from C₁-C₆alkyl and C₁-C₆haloalkyl;
R₇ is cyclopropyl, cyclobutyl, cyclopentyl, cyclohexyl or C₃-C₆alkyl; and
R₁₀ and R₁₁ are independently chosen from:
(i) hydrogen; and
(ii) C₁-C₆alkyl, C₂-C₆alkenyl, C₂-C₆alkynyl, (C₃-C₈cycloalkyl)C₀-C₂alkyl, and groups that are taken together to form a 4- to 7-membered heterocycloalkyl, each of which is substituted with from 0 to 4 substituents independently chosen from oxo, amino, cyano, C₁-C₄alkyl mono- or di-(C₁-C₄alkyl)amino.

18. A compound or salt or hydrate thereof according to claim 5, wherein the compound satisfies the formula: 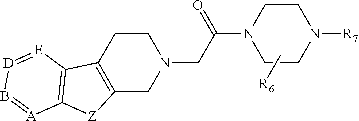
wherein:
Z is oxygen, sulfur or NR₃;
A, B, D and E are independently nitrogen or CR₁; such that exactly zero, one or two of A, B, D and E are nitrogen; wherein each R₁ is independently chosen from hydrogen, halogen, cyano, C₁-C₆alkyl, C₁-C₆haloalkyl, (C₃-C₈cycloalkyl)C₀-C₂alkyl, C₂-C₆alkyl ether, C₁-C₆alkoxy, C₁-C₆alkanoyl, mono- or di-(C₁-C₆alkyl)amino and mono- or di-(C₁-C₆alkyl)aminocarbonyl;
R₆ represents from 0 to 2 substituents independently chosen from C₁-C₆alkyl and C₁-C₆haloalkyl; and
R₇ is cyclopropyl, cyclobutyl, cyclopentyl, cyclohexyl or C₃-C₆alkyl.

19. A compound or salt or hydrate thereof according to claim 5, wherein the compound satisfies the formula: 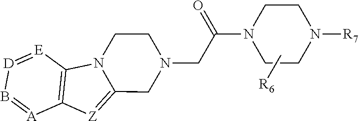
wherein:
Z is nitrogen or CR₃;
A, B, D and E are independently nitrogen or CR₁; such that exactly zero, one or two of A, B, D and E are nitrogen; wherein each R₁ is independently chosen from hydrogen, halogen, cyano, C₁-C₆alkyl, C₁-C₆haloalkyl, (C₃-C₈cycloalkyl)C₀-C₂alkyl, C₂-C₆alkyl ether, C₁-C₆alkoxy, C₁-C₆alkanoyl, mono- or di-(C₁-C₆alkyl)amino and mono- or di-(C₁-C₆alkyl)aminocarbonyl
R₆ represents from 0 to 2 substituents independently chosen from C₁-C₆alkyl and C₁-C₆haloalkyl; and
R₇ is cyclopropyl, cyclobutyl, cyclopentyl, cyclohexyl or C₃-C₆alkyl.

20.-32. (canceled)

33. A compound or salt or hydrate thereof according to claim 1, wherein the compound is capable of exhibiting a Kᵢ value of 1 micromolar or less, as determined using an assay for H3 receptor GTP binding.

34. A compound or salt or hydrate thereof according to claim 33, wherein the compound is capable of exhibiting a Kᵢ value of 100 nanomolar or less, as determined using an assay for H3 receptor GTP binding.

35. A pharmaceutical composition, comprising at least one compound or salt according to any one of claims 1-34 in combination with a physiologically acceptable carrier or excipient.

36. A pharmaceutical composition according to claim 35 wherein the composition is formulated as an injectable fluid, an aerosol, a cream, a gel, a pill, a capsule, a syrup or a transdermal patch.

37. A method for treating a condition responsive to H3 receptor modulation in a patient, comprising administering to the patient a therapeutically effective amount of a compound or salt or hydrate thereof according to claim 1, and thereby alleviating the condition in the patient.

38. (canceled)

39. A method according to claim 37, wherein the condition is attention deficit disorder, attention deficit hyperactivity disorder, dementia, schizophrenia, a cognitive disorder, epilepsy, migraine, excessive daytime sleepiness, shift work sleep disorder, jet lag, fatigue or a fatigue-related disorder, narcolepsy, sleep apnea, allergic rhinitis, vertigo, motion sickness, a memory disorder, or Parkinson's disease.

40. A method according to claim 37, wherein the condition is obesity, an eating disorder or diabetes.

41. A method according to claim 37, wherein the patient is a human.

42.-44. (canceled)

45. A packaged pharmaceutical preparation, comprising:
(a) a pharmaceutical composition according to claim 35 in a container; and
(b) instructions for using the composition to treat a condition responsive to H3 receptor modulation in a patient.

46. A packaged pharmaceutical preparation according to claim 45, wherein the condition is attention deficit disorder, attention deficit disorder, attention deficit hyperactivity disorder, dementia, schizophrenia, a cognitive disorder, epilepsy, migraine, excessive daytime sleepiness, shift work sleep disorder, jet lag, fatigue or a fatigue-related disorder, narcolepsy, sleep apnea, allergic rhinitis, vertigo, motion sickness, a memory disorder, or Parkinson's disease.

47. A packaged pharmaceutical preparation according to claim 45, wherein the condition is obesity, an eating disorder or diabetes.

48.-50. (canceled)

51. A compound or salt or hydrate thereof according to claim 1, wherein the compound is:
1-(4-cyclobutyl-piperazin-1-yl)-2-(1,2-dihydro-4H-3,8a,9-triaza-fluoren-3-yl)-ethanone;
1-(4-cyclobutyl-piperazin-1-yl)-2-(2-methyl-5,8-dihydro-6H-pyrido[4′,3′:4,5]thieno[2,3-d]pyrimidin-7-yl)-ethanone;
1-(4-cyclobutyl-piperazin-1-yl)-2-(3,4-dihydro-1H-benzo[4,5]furo[2,3-c]pyridin-2-yl)-ethanone;
1-(4-cyclobutyl-piperazin-1-yl)-2-(3,4-dihydro-1H-benzo[4,5]thieno[2,3-c]pyridin-2-yl)-ethanone;
1-(4-cyclobutyl-piperazin-1-yl)-2-(5,8-dihydro-6H-pyrido[4′,3′:4,5]thieno[2,3-d]pyrimidin-7-yl)-ethanone;
2-[2-(1,3′-bipyrrolidin-1′-yl)-2-oxoethyl]-2,3,4,9-tetrahydro-1H-beta-carboline;
2-[2-(1,4′-bipiperidin-1′-yl)-2-oxoethyl]-2,3,4,9-tetrahydro-1H-beta-carboline;
2-[2-(2-methyl-1,4′-bipiperidin-1′-yl)-2-oxoethyl]-2,3,4,9-tetrahydro-1H-beta-carboline;
2-[2-(4-allylpiperazin-1-yl)-2-oxoethyl]-2,3,4,9-tetrahydro-1H-beta-carboline;
2-[2-(4-butyl-1,4-diazepan-1-yl)-2-oxoethyl]-2,3,4,9-tetrahydro-1H-beta-carboline;
2-[2-(4-butylpiperazin-1-yl)-2-oxoethyl]-2,3,4,9-tetrahydro-1H-beta-carboline;
2-[2-(4-cyclobutyl-1,4-diazepan-1-yl)-2-oxoethyl]-2,3,4,9-tetrahydro-1H-beta-carboline;
2-[2-(4-cyclobutylpiperazin-1-yl)-2-oxoethyl]-2,3,4,9-tetrahydro-1H-beta-carboline;
2-[2-(4-cyclobutylpiperazin-1-yl)-2-oxoethyl]-2,3,4,9-tetrahydro-1H-beta-carboline-6-carbonitrile;
2-[2-(4-cyclobutylpiperazin-1-yl)-2-oxoethyl]-5-fluoro-2,3,4,9-tetrahydro-1H-beta-carboline;
2-[2-(4-cyclobutylpiperazin-1-yl)-2-oxoethyl]-6-fluoro-2,3,4,9-tetrahydro-1H-beta-carboline;
2-[2-(4-cyclobutylpiperazin-1-yl)-2-oxoethyl]-6-fluoro-9-methyl-2,3,4,9-tetrahydro-1H-beta-carboline;
2-[2-(4-cyclobutylpiperazin-1-yl)-2-oxoethyl]-6-methoxy-2,3,4,9-tetrahydro-1H-beta-carboline;
2-[2-(4-cyclobutylpiperazin-1-yl)-2-oxoethyl]-6-methoxy-9-methyl-2,3,4,9-tetrahydro-1H-beta-carboline;
2-[2-(4-cyclobutylpiperazin-1-yl)-2-oxoethyl]-6-methyl-2,3,4,9-tetrahydro-1H-beta-carboline;
2-[2-(4-cyclobutylpiperazin-1-yl)-2-oxoethyl]-6-pyrimidin-5-yl-2,3,4,9-tetrahydro-1H-beta-carboline;
2-[2-(4-cyclobutylpiperazin-1-yl)-2-oxoethyl]-7-fluoro-2,3,4,9-tetrahydro-1H-beta-carboline;
2-[2-(4-cyclobutylpiperazin-1-yl)-2-oxoethyl]-7-methoxy-2,3,4,5-tetrahydro-1H-pyrido[4,3-b]indole;
2-[2-(4-cyclobutylpiperazin-1-yl)-2-oxoethyl]-7-pyrimidin-5-yl-2,3,4,9-tetrahydro-1H-beta-carboline;
2-[2-(4-cyclobutylpiperazin-1-yl)-2-oxoethyl]-8-fluoro-2,3,4,9-tetrahydro-1H-beta-carboline;
2-[2-(4-cyclobutylpiperazin-1-yl)-2-oxoethyl]-8-methoxy-1,2,3,4-tetrahydropyrazino[1,2-a]indole;
2-[2-(4-cyclobutylpiperazin-1-yl)-2-oxoethyl]-9-(2,2,2-trifluoroethyl)-2,3,4,9-tetrahydro-1H-beta-carboline;
2-[2-(4-cyclobutylpiperazin-1-yl)-2-oxoethyl]-9-ethyl-2,3,4,9-tetrahydro-1H-beta-carboline;
2-[2-(4-cyclobutylpiperazin-1-yl)-2-oxoethyl]-9-isopropyl-2,3,4,9-tetrahydro-1H-beta-carboline;
2-[2-(4-cyclobutylpiperazin-1-yl)-2-oxoethyl]-9-methyl-2,3,4,9-tetrahydro-1H-beta-carboline;
2-[2-(4-cyclobutylpiperazin-1-yl)-2-oxoethyl]-9-phenyl-2,3,4,9-tetrahydro-1H-beta-carboline;
2-[2-(4-cyclobutylpiperazin-1-yl)-2-oxoethyl]-9-propyl-2,3,4,9-tetrahydro-1H-beta-carboline;
2-[2-(4-cyclohexylpiperazin-1-yl)-2-oxoethyl]-2,3,4,9-tetrahydro-1H-beta-carboline;
2-[2-(4-cyclopentylpiperazin-1-yl)-2-oxoethyl]-2,3,4,9-tetrahydro-1H-beta-carboline;
2-[2-(4-ethylpiperazin-1-yl)-2-oxoethyl]-2,3,4,9-tetrahydro-1H-beta-carboline;
2-[2-(4-isobutylpiperazin-1-yl)-2-oxoethyl]-2,3,4,9-tetrahydro-1H-beta-carboline;
2-[2-(4-isopropyl-1,4-diazepan-1-yl)-2-oxoethyl]-2,3,4,9-tetrahydro-1H-beta-carboline;
2-[2-(4-isopropylpiperazin-1-yl)-2-oxoethyl]-2,3,4,9-tetrahydro-1H-beta-carboline;
2-[2-(4-propylpiperazin-1-yl)-2-oxoethyl]-2,3,4,9-tetrahydro-1H-beta-carboline;
2-[2-(6-methyloctahydro-2H-pyrido[1,2-a]pyrazin-2-yl)-2-oxoethyl]-2,3,4,9-tetrahydro-1571H-beta-carboline;
2-[2-(octahydro-2H-pyrido[1,2-a]pyrazin-2-yl)-2-oxoethyl]-2,3,4,9-tetrahydro-1H-beta-carboline;
2-[2-oxo-2-(4-pyrrolidin-1-ylpiperidin-1-yl)ethyl]-2,3,4,9-tetrahydro-1H-beta-carboline;
2-{2-[4-(2-methylcyclopentyl)piperazin-1-yl]-2-oxoethyl}-2,3,4,9-tetrahydro-1H-beta-carboline;
2-{2-oxo-2-[4-(1-phenylethyl)piperazin-1-yl]ethyl}-2,3,4,9-tetrahydro-1H-beta-carboline;
2-{2-oxo-2-[4-(2-piperidin-1-ylethyl)piperazin-1-yl]ethyl}-2,3,4,9-tetrahydro-1H-beta-carboline;
2-{2-oxo-2-[4-(2-pyrrolidin-1-ylethyl)piperazin-1-yl]ethyl}-2,3,4,9-tetrahydro-1H-beta-carboline;
2-{2-oxo-2-[4-(3-piperidin-1-ylpropyl)piperazin-1-yl]ethyl}-2,3,4,9-tetrahydro-1H-beta-carboline;
2-{2-oxo-2-[4-(3-pyrrolidin-1-ylpropyl)piperazin-1-yl]ethyl}-2,3,4,9-tetrahydro-1H-beta-carboline;
2-{2-oxo-2-[4-(tetrahydro-2H-pyran-4-yl)piperazin-1-yl]ethyl}-2,3,4,9-tetrahydro-1H-beta-carboline;
6-[2-(4-cyclobutylpiperazin-1-yl)-2-oxoethyl]-5,6,7,8-tetrahydropyrazolo[1,5-a]pyrido[3,4-d]pyrimidine
6-bromo-2-[2-(4-cyclobutylpiperazin-1-yl)-2-oxoethyl]-2,3,4,9-tetrahydro-1H-beta-carboline;
7-[2-(4-cyclobutyl-piperazin-1-yl)-2-oxo-ethyl]-3-methyl-5,6,7,8-tetrahydro-3H-pyrido[4′,3′:4,5]thieno[2,3-d]pyrimidin-4-one;
7-[2-(4-cyclobutylpiperazin-1-yl)-2-oxoethyl]-5,6,7,8-tetrahydropyrazolo[1,5-a]pyrido[4,3-d]pyrimidine;
7-[2-(4-cyclobutylpiperazin-1-yl)-2-oxoethyl]-9-methyl-6,7, 8,9-tetrahydro-5H-pyrido[4′,3′:4,5]pyrrolo[2,3-b]pyridine;
N-(1-methylpiperidin-4-yl)-N-ethyl-2-(1,3,4,9-tetrahydro-2H-beta-carbolin-2-yl)acetamide;
N-(1-methylpiperidin-4-yl)-N-propyl-2-(1,3,4,9-tetrahydro-2H-beta-carbolin-2-yl)acetamide;
N-(2-methoxyethyl)-N-(1-methylpiperidin-4-yl)-2-(1,3,4,9-tetrahydro-2H-beta-carbolin-2-yl)acetamide;
N-(cyclopropylmethyl)-N-propyl-1-(1,3,4,9-tetrahydro-2H-beta-carbolin-2-ylacetyl)piperidin-4-amine;
N,N-diethyl-1-(1,3,4,9-tetrahydro-2H-beta-carbolin-2-ylacetyl)piperidin-4-amine;
N,N-diethyl-1-(1,3,4,9-tetrahydro-2H-beta-carbolin-2-ylacetyl)pyrrolidin-3-amine;
N-[2-(dimethylamino)ethyl]-N-methyl-2-(1,3,4,9-tetrahydro-2H-beta-carbolin-2-yl)acetamide;
N-[3-(dimethylamino)propyl]-N-(1-methylpiperidin-4-yl)-2-(1,3,4,9-tetrahydro-2H-beta-carbolin-2-yl)acetamide; or
N-methyl-N-(1-ethylpiperidin-4-yl)-2-(1,3,4,9-tetrahydro-2H-beta-carbolin-2-yl)acetamide.
N,N-diethyl-1-(1,3,4,9-tetrahydro-2H-beta-carbolin-2-ylacetyl)piperidin-4-amine;
N,N-diethyl-1-(1,3,4,9-tetrahydro-2H-beta-carbolin-2-ylacetyl)pyrrolidin-3-amine;
N-[2-(dimethylamino)ethyl]-N-methyl-2-(1,3,4,9-tetrahydro-2H-beta-carbolin-2-yl)acetamide;
N-[3-(dimethylamino)propyl]-N-(1-methylpiperidin-4-yl)-2-(1,3,4,9-tetrahydro-2H-beta-carbolin-2-yl)acetamide; or
N-methyl-N-(1-ethylpiperidin-4-yl)-2-(1,3,4,9-tetrahydro-2H-beta-carbolin-2-yl)acetamide.
